(19) United States
(12) Patent Application Publication
(10) Pub. No.: US 2020/0010519 A1
(43) Pub. Date: Jan. 9, 2020

(54) NUCLEASE FUSIONS FOR ENHANCING GENOME EDITING BY HOMOLOGY-DIRECTED TRANSGENE INTEGRATION

(71) Applicants: INSTITUT NATIONAL DE LA SANTE ET DE LA RECHERCHE MEDICALE(INSERM), PARIS (FR); CENTRE NATIONAL DE LA RECHERCHE SCIENTIFIQUE, PARIS CEDEX 16 (FR); MUSEUM NATIONAL D'HISTOIRE NATURELLE, PARIS (FR); UNIVERSITÉ DE NANTES, NANTES (FR)

(72) Inventors: Ignacio ANEGON, NANTES (FR); Marine CHARPENTIER, PARIS (FR); Jean-Paul CONCORDET, PARIS (FR); Carine GIOVANNANGELLI, PARIS (FR); Bernard LOPEZ, VILLEJUIF (FR)

(21) Appl. No.: 16/492,221

(22) PCT Filed: Mar. 9, 2018
(86) PCT No.: PCT/EP2018/055883

§ 371 Date: Sep. 9, 2019

(30) Foreign Application Priority Data
Mar. 10, 2017 (EP) 17305260.6

(51) Int. Cl. C07K 14/47; C12N 15/86; C12N 9/22; C12N 9/24; C12N 15/11; C12N 15/90

(52) U.S. Cl. CPC C07K 14/4702; C07K 14/4738; C12N 15/86; C12N 9/22; C07K 2319/70; C12N 15/111; C12N 15/907; C07K 2319/09; C12N 9/24

(57) ABSTRACT

The present invention relates to nuclease protein fusions for enhancing genome editing by homology-directed transgene integration (HDI). The inventors found that the rate of HDI mediated by the CRISPR/Cas9 system may be substantially improved by providing the Cas9 nuclease in the form of a fusion protein with at least the N-terminal domain of the CtIP protein. CtIP proteins are involved in the early steps of homologous recombination. In addition, the inventors identified the subdomains of the N-terminal domain of the CtIP protein that are important for improving the HDI rate. Thus, the invention relates to fusion proteins comprising a Cas9 protein, a tetramerization domain of a CtIP protein and a dimerization domain of a CtIP protein. Particularly, the inventors have tested these fusion proteins HEK293 cells, RG37DR cells and Sprague-Dawley rats.

### FIELD OF THE INVENTION

The present invention relates to nuclease protein fusions, and especially to Cas9 nuclease fusions, for enhancing genome editing by homology-directed transgene integration.

In particular, the invention relates to a fusion protein between a Cas9 nuclease and the N-terminal domain of a CtIP protein, comprising a dimerization domain and a tetramerization domain.

### BACKGROUND OF THE INVENTION

Early studies in yeast using Homing Endonuclease I-SceI established the main principles of genome editing (Dujon, 1989; Plessis et al., 1992). In pioneer studies with mammalian cells, the induction of a double strand break (DSB) at a unique position, again using the homing endonuclease I-SceI, allowed precise sequence modification by homologous recombination (HR) (Rouet et al., 1994).

Subsequently, different artificial sequence-specific nucleases, such as zinc finger nucleases, TALE Nucleases and more recently Clustered Regularly Interspaced Palindromic Repeats/CRISPR associated protein 9 (CRISPR/Cas9), have been used to introduce a DSB at a target locus in order to edit the genome (Deltcheva et al., 2011; Doyon et al., 2008; Huang et al., 2011).

Different DNA DSB repair systems can come into play after target DNA cleavage and determine the nature of genome editing. Classical Non-Homologous End Joining (cNHEJ) and micro-homology-mediated end joining (MMEJ) mediate ligation of DNA ends and result in small targeted but un-programmed deletions/insertions that allow to efficiently inactivating gene coding sequences.

On the other hand, homologous Recombination (HR) is only active during S/G2 phases of the cell cycle when homologous template DNA is available for repair. Artificial donor DNA with homology arms to the target DNA can also serve as a template, allowing precise genome editing, such as transgene integration.

In order to favour homology-dependent transgene integration (herein designated as HDI) following target DNA cleavage over NHEJ, different strategies have been developed.

For example, when cells are synchronized in S/G2 phases, HDI can be improved up to 5 fold (Yang et al., 2016). However, cells synchronization may be tricky to perform, and in particular may often result in unwanted perturbations of cells physiological mechanisms. In addition, one major drawback of this method is that synchronization of cells may not be suitable when cells are targeted in vivo.

Other reported that NHEJ may be inhibited through inactivation of Ligase 4 activity, which consequently improves HDI (Gandia et al., 2016).

Some other approaches consisted in engineering protein fusions with a catalytic inactivated Cas9 protein (e.g. dCas9).

Moreover, Chaikind et al. (2016) disclosed a programmable dCas9-serine recombinase fusion protein, based on inactive dCas9 and Ginβ. However, this system operates on site specific recombinase sites, which substantially limit its use.

Another approach has been developed using Geminin (Gutschner et al., 2016). Part of Geminin was fused to the catalytic active human Cas9 nuclease. Geminin is a natural substrate of the APC/Cdh1 complex, which is the major cell-cycle controlling E3 ubiquitin ligase, and is consequently degraded during G1 phase. When using Cas9-Geminin nuclease, the fusion protein is proteolized in late M and G1 phase, whereas the fusion protein accumulates during the S/G2/M phases. Consequently, HDI rate is improved and the rate of non-programmed mutations induced by NHEJ is decreased (Howden et al., 2016). In other words, this approach is based on an artificial modulation of the presence of Cas9 protein within defined phases of the cell cycle.

Therefore, there is a need to provide new tools to enhance HDI, in particular tools that maintain the activity of Cas9 unaltered and can be performed without altering the overall cellular physiology.

### SUMMARY OF THE INVENTION

One aspect of the invention relates to a fusion protein comprising at least (a) a nuclease, (b) a dimerization domain of a CtIP protein and (c) a tetramerization domain of a CtIP protein, with the proviso that the said fusion protein does not comprise the full length CtIP protein.

This invention notably pertains to a fusion protein comprising at least (a) a Cas protein, (b) a dimerization domain of a CtIP protein and (c) a tetramerization domain of a CtIP protein, with the proviso that the said fusion protein does not comprise the full length CtIP protein.

In another aspect, the invention also relates to a nucleic acid encoding a fusion protein as defined herein.

Another aspect of the invention relates to a nucleic acid vector for recombinant protein expression comprising a nucleic acid as described herein.

A further aspect of the invention relates to a delivery particle comprising a fusion protein, a nucleic acid or a nucleic acid vector according to the description herein.

The invention also relates to a fusion protein, a nucleic acid, a nucleic acid vector or a delivery particle as described herein for use as a medicament.

In another aspect, the invention also relates to a host cell comprising a fusion protein, a nucleic acid or a nucleic acid vector as described herein.

The invention further relates to a pharmaceutical composition comprising (i) a fusion protein, a nucleic acid, a nucleic acid vector or a delivery particle as described herein, and (ii) a pharmaceutically acceptable vehicle.

Another aspect of the invention also relates to a pharmaceutical composition as described herein for use as an active agent for editing the genome into at least one target cell.

Another aspect of the invention relates to a method for editing a genome into at least one target cell comprising at least the step of administering to an individual in need thereof a pharmaceutical composition as described herein.

Finally, the invention further relates to a kit for editing the genome of at least a target cell, comprising:
a fusion protein, a nucleic acid encoding the said fusion protein, a nucleic acid vector comprising the said nucleic acid or a delivery particle comprising the said fusion protein according to the description herein; and
one or more site-specific guide RNAs (gRNAs) or a nucleic acid vector for expressing one or more site specific guide RNAs (gRNAs).

### LEGENDS OF THE FIGURES

FIG. 1. Scheme illustrating the overall strategy to perform integration of a GFP transgene at the Rosa26 locus of a rat genome. PCRs performed to genotype rat embryos following microinjection into rat eggs. The PCR donor integration scheme shows the two PCRs events used to identify the animals that harbour the donor sequence irrespectively on whether the insertion is in the Rosa26 locus following DNA cleavage by Cas9-HE or Cas9. The PCR in-out scheme shows the two PCRs events used to analyse whether the insertion has occurred into the Rosa26 locus, since at both 5′ and 3′ extremities there are external oligos corresponding to genomic sequences that are beyond the homology arms of the donor sequence (Rosa26-5outFor (SEQ ID NO. 38) and Rosa26-3outRev (SEQ ID NO. 41)). PCRs using primers rROSAfwl (SEQ ID NO. 37) and rROSArevl (SEQ ID NO. 42) allowed identifying embryos with no donor DNA insertion but with NHEJ.

FIG. 2. Plot illustrating how the recruitment of CtIP at the cleavage site stimulates HDI in RG37DR cells. The relative rate of HDI (black bars) and the relative mutation rate grey bars) are obtained by the T7 test, induced by Cas9 that directly recruits CtIP at the DSB site by fusion. The data shown are representative of six independent experiments. Results are expressed as mean of relative HDI rate calculated by normalizing every HDI rate by the HDI rate induced by Cas9. Asterisks indicate that difference is statistically significant when comparing Cas9 to Cas9-CtIP (P<0.05) after t-test.

FIG. 3. Functional study of HDI stimulation by systematically truncated CtIP mutants and fusing every part to Cas9.

(A) Schematic diagram of CtIP protein showing known features and different truncated CtIP protein that have been fused to Cas9, namely 1-149 (SEQ ID NO. 5), 1-296 (SEQ ID NO. 6), 1-416 (SEQ ID NO. 7), 1-669 (SEQ ID NO. 8), 416-897 (SEQ ID NO.

10), 669-897 (SEQ ID NO. 11) and 1-790 (SEQ ID NO. 9).

(B) Plot illustrating the relative rate of HDI (black bars) and the relative mutation rate obtained by the T7 test (grey bars), induced by the different Cas9-CtIP fusions as described in (A). The data shown are representative of four independent experiments. Results are expressed as mean of HDI rate calculated by normalizing HDI rates by the HDI rate induced by Cas9. Asterisks indicate that the difference is statistically significant when comparing Cas9 to Cas9-CtIP derivatives (P<0.05) after t-test.

FIG. 4. Functional analysis of HE domain of CtIP.

(A) Schematic diagram of the HE (1-296; SEQ ID NO. 6) domain showing known features and phosphorylation sites of CtIP (S233, T245 and S276) and different truncated HE domains that have been fused to Cas9, namely HE1 (SEQ ID NO. 12), HE2 (SEQ ID NO. 13), HE3 (SEQ ID NO. 14), HE(3E) (SEQ ID NO. 15) and HE(3A)

(SEQ ID NO. 16).

(B) Plot illustrating the relative rate of HDI (black bars) and the relative mutation rate obtained by the T7 test (grey bars), induced by Cas9 fusions to different HE domains, i.e. HE1 (SEQ ID NO. 12), HE2 (SEQ ID NO. 13), HE3 (SEQ ID NO. 14). The data shown are representative of five independent experiments. Results are expressed as mean of relative HDI rate calculated by normalizing every HDI rate by the HDI rate induced by Cas9. Asterisks indicate that difference is statistically significant when comparing Cas9 to Cas9-HE derivatives (P<0.05) after t-test.

(C) Plot illustrating the Western blotting analysis with anti-Cas9 and anti-tubulin antibodies of transfected cells.

(D) Plot illustrating the relative rate of HDI (black bars) and the relative mutation rate obtained by the T7 test (grey bars), induced by Cas9 that directly recruit different HE mutant for CDK phosphorylation site, at the T2 cut site by fusion, i.e. HE(3E) (SEQ ID NO. 15) and HE(3A) (SEQ ID NO. 16). The data shown are representative of six independent experiments. Results are expressed as mean of relative HDI rate calculated by normalizing every HDI rate by the HDI rate induced by Cas9. Asterisks indicate that difference is statistically significant when comparing Cas9 to Cas9-HE derivatives (P<0.05) after t-test.

(E) Plot illustrating the Western blotting analysis with anti-Cas9 and anti-tubulin antibodies of transfected cells.

FIG. 5. Comparison of Cas9-HE and Cas9-Geminin fusion proteins activities.

(A) Plot illustrating the relative rate of HDI (black bars) and the relative mutation rate obtain by the T7 test (grey bars), induced by Cas9-HE fusion protein (C9-HE), Cas9-Geminin fusion protein (C9-Geminin) and Cas9-HE-Geminin fusion protein (C9-HE-Geminin) at the cleavage site. The data shown are representative of six independent experiments. Results are expressed as mean of relative HDI rate calculated by normalizing every HDI rate by the HDI rate induced by Cas9. Asterisks indicate that difference is statistically significant when comparing Cas9 to Cas9-HE derivatives (P<0.05) after t-test.

(B) Plot illustrating the Western blotting analysis with anti-Cas9 and anti-tubulin antibodies of transfected cells.

FIG. 6. RPA foci formation after X-ray irradiation.

RPA foci were counted in control cells and at different times after X-ray irradiation in RG37 cells transfected with the indicated Cas9 fusions or anti-CtIP siRNA or control siRNA. Counts of RPA foci per nucleus are cumulated from three independent transfection experiments.

(A) Plot illustrating the counts of RPA foci per nucleus are shown at 6 hours after irradiation, which corresponds to the peak of RPA foci per nucleus after irradiation. Median number of foci per nucleus is indicated as a bar. Silencing CtIP expression diminished RPA foci number per cell compared to control cells and cells transfected with control siRNA (***, p<0.0005; ****, p<0.0001, nonparametric Mann-Whitney t-test) as expected while no difference was found between cells with Cas9, Cas9-CtIP or Cas9-HE.

(B-G) Plot illustrating the counts of RPA foci per nucleus of control cells are shown at the indicated times after irradiation. Median number of foci per nucleus is indicated as a bar.

FIG. 7. HDR stimulation by the HE domain takes place at different target genes and can depend on the guide RNA used.

(A) Relative frequencies of HDR induced by Cas9-HE were compared to those induced by Cas9 at 5 different target genes in HEK293 cells using previously published guide RNAs and donor plasmids (Savic et al.; 2015). Targeted integration of donor plasmid results in in frame-insertion of E2A-neoR cDNA. G418 (neomycin)-resistant colonies were counted after Cresyl violet staining to measure HDR-mediated events and normalized by the number of colonies obtained with Cas9 to give the relative HDR frequencies indicated. Data represented is from 3 independent experiments for TGIF2, RAD21, and CREB genes and from 4 for ATF4 and GABP genes. Error bars indicate standard deviation.

(B) Relative frequencies of HDR induced by Cas9-HE were compared to those induced by Cas9 with the indicated guide RNAs, which all target cleavage to a small 50 bp region of the AAVS1 locus, and a common p84Δ donor plasmid, harbouring approximately 800 bp homology arms. Asterisks indicate that difference is statistically significant when comparing Cas9-HE to Cas9 in t-test (*, P<0.05). Data represented is from 5 independent experiments.

### DETAILED DESCRIPTION OF THE INVENTION

The inventors provide herein a novel and simple approach to improve HDI using CRISPR/Cas9 system, in which the Cas9 nuclease is fused to a N-terminal domain of the CtIP protein, which is a key protein in early steps of HR. The approach described herein is straightforward, does not require using genetically modified cells or pharmacological reagents, and allows obtaining up to 3 fold higher HDI rate using donor

DNA.

Fusions between the CtIP protein and a nuclease have been previously disclosed in the art, such as, e.g. patent applications WO 2012/138939, WO 2015/153889 and WO 2016/054326. However, these fusions are based upon a fusion between the full length CtIP protein and a nuclease.

Surprisingly, the inventors have shown that upon cleavage of a target DNA by the CRISPR/Cas9 system in order to create a double strand break (DSB), recruitment of CtIP protein at the DSB site promotes homologous recombination at a high rate, in the presence of a donor DNA. Therefore, CRISPR/Cas9-based genome editing, e.g. site directed genome deletions or site-directed genome insertions, may be successfully performed by the use of a fusion protein involving the Cas9 nuclease and at least the N-terminal domain of a CtIP protein.

Without wishing to be bound to a theory, the inventors consider that fusion proteins with the N-terminal domain of a CtIP protein may be engineered for any other type of nuclease involved in genome editing, such as, e.g. zinc-finger nucleases (ZFNs), transcription-activator like effector nucleases (TALENs) and meganucleases.

As it will emerge from the description and the examples below, the N-terminal domain of the CtIP protein may comprise a dimerization domain and a tetramerization domain of the CtIP protein, and optionally a domain comprising one or more CDK phosphorylation sites.

### Fusion Proteins

The invention relates to a fusion protein comprising at least (a) a nuclease and (b) a N-terminal domain of a CtIP protein.

The invention further relates to a fusion protein comprising at least (a) a nuclease and (b) a domain of a CtIP protein consisting of the N-terminal domain of a CtIP protein.

The invention relates to a fusion protein comprising at least (a) a nuclease, (b) a dimerization domain of a CtIP protein and (c) a tetramerization domain of a CtIP protein. In some embodiments, the fusion protein according to the instant invention may be characterized by the fact that the said fusion protein does not comprise the full length CtIP protein.

This invention notably concerns a fusion protein comprising at least (a) a Cas protein, (b) a dimerization domain of a CtIP protein and (c) a tetramerization domain of a CtIP protein.

Within the scope of the instant invention, the term “fusion protein” refers to a polypeptide made up with 2 or more domains originating from distinct polypeptide sources.

Within the scope of the invention, a nuclease according to the invention may be a “programmable nuclease”, which refers to a nuclease that can be programmed to recognize and edit a predetermined location in a DNA sequence, in particular a genome, of a target cell.

In some embodiments, the nuclease is selected in a group comprising a Cas nuclease, a zinc-finger nuclease (ZFN), transcription-activator like effector nuclease (TALEN) and a meganuclease, preferably a Cas nuclease.

### Cas Nucleases

In certain embodiments, the Cas nuclease is selected in a group comprising a class I Cas nuclease, a class II Cas nuclease and a class III Cas nuclease.

Class I, class II or class III Cas nucleases have been in particular described in Chylinski et al. (2014); Sinkunas et al. (2011); Aliyari et al. (2009); Cass et al. (2015), Makarova et al. (2011); Gasiunas et al. (2012) ; Heler et al. (2015); Esvelt et al. (2013), Zetsche et al. (2015), and Chylinski et al. (2013).

In some embodiments, a class I Cas nuclease is selected in a group comprising Cas3, Cas8a, Cas8b, Cas8c, Cas10d, Csel and Csy1.

In some embodiments, a class II Cas nuclease is selected in a group comprising Cas4, Cas9, Cpf1 and Csn2.

In some embodiments, a class III Cas nuclease is selected in a group comprising Cas10, Cmr5 and Csm2.

In some embodiments, the Cas nuclease is a Cas9 nuclease or a Cpfl nuclease.

In some embodiments, the Cas9 protein may originate from a bacterial source, in particular a bacterium selected in a group comprising Acaryochloris marina, Actinomyces naeslundii, Alcanivorax dieselolei, Belliella baltica, Campylobacter jejuni, Corynebacterium diphtheriae, Coriobacterium glomerans, Corynebacterium ulcerans, Desulfomonile tiedjei, Dickeya dadantii, Escherichia coli, Francisella tularensis, Lactobacillus kefiranofaciens, Listeria innocua, Methylobacterium extorquens, Micrococcus luteus, Myxococcus fulvus, Neisseria meningitidis, Pasteurella multocida, Prevotella intermedia, Prochlorococcus marinus, Psychroflexus torquis, Sphaerobacter thermophilus, Sphingobacterium sp., Staphylococcus aureus, Streptococcus mutans, Streptococcus pneumoniae, Streptococcus pyogenes, Streptococcus thermophilus and Streptomyces bingchenggensis.

In some embodiments, the Cas9 protein may originate from an archaebacterial source, such as e.g. Methanoculleus bourgensis.

Without any limitation, the Cas9 protein disclosed herein encompasses homologs, paralogs and orthologs and variants of naturally occurring Cas9 proteins.

In certain embodiments, the Cas9 variants may include SpCas9-HF1 (Kleinstiver et al.; 2016); fCas9, which is a fusion of catalytically inactive Cas9 to Fokl nuclease (Guilinger et al.; 2014), and any rationally engineered Cas9 nucleases with improved specificity as disclosed by Slaymaker et al. (2016) and Kleinstiver et al. (2016) or any rationally engineered Cas9 nuclease with altered PAM specificity as disclosed by Kleinstiver et al. (2016).

In some embodiments, the Cas9 protein originates from Streptococcus pyogenes serotype M1 (SEQ ID NO. 1).

### Zinc Finger Nucleases (ZFNs)

Within the scope of the invention, a ZFN refers to a protein comprising a zinc finger domain with specific binding affinity for a desired specific target sequence.

In a non-limitative manner, ZFN and vectors which are suitable for the invention are described in e.g. EP 2368982.

Zinc finger nucleases, principles and methods suitable for implementing the invention have been extensively described, e.g. Wood et al. (2011); Miller et al. (2007); Urnov et al. (2010); Perez et al. (2008).

### TALE Nucleases (TALENs)

Within the scope of the invention, a TALEN refers to an artificial nuclease made up by the fusion of a transcriptional activator like effector DNA binding domain and a DNA cleavage domain, e.g, a FokI domain.

In a non-limitative manner, the principles and methods for using TALENs have been extensively described, e.g. in Wood et al. (2011); Bedell et al. (2012); Joung and Sander (2013); Reyon et al. (2012); Ding et al. (2013) and Miller et al. (2011).

### CtIP Protein and Domains Thereof

Within the scope of the invention, a CtIP protein (CtBP Interacting protein) according to the invention may also be known in the in art as retinoblastoma-binding protein 8, RBBP-8, SAE2, RIM, DNA endonuclease RBBP 8, Seckel syndrome 2, SCKL2, COM1 and JWDS. It is to be noted that the endonuclease activity of the CtIP protein is still in debate.

The CtIP protein is a protein that cooperates with the MRE11-RAD5O-NBN (MRN) complex in processing meiotic and mitotic double-strand breaks (DSBs) by ensuring both resection and intra-chromosomal association of the broken ends.

The CtIP proteins are highly conserved among species and the high conservation of CtIP proteins concerns in particular its N-terminal domain, which encompasses a dimerization domain, a tetramerization domain and CDK phosphorylation sites. Moreover, the tetramerization domain may also be involved in the binding properties of CtIP proteins to the MRN complex.

For example, human CtIP protein is a 897 amino acids protein of sequence SEQ ID NO. 2.

Within the scope of the instant invention, the “N-terminal domain of a CtIP protein” is intended to refer to the domain of a CtIP protein comprising from amino acid 1 to amino acid 296 (1-296 aa) of the said CtIP protein, in particular an amino acids sequence SEQ ID NO. 6. The N-terminal domain of the CtIP protein represented by amino acid 1 to amino acid 296 (1-296 aa) is referred herein as the “HE” domain of the CtIP protein.

The expression “dimerization domain of a CtIP protein” refers to a continuous sequence of amino acids of a CtIP protein involved in the formation of dimers between two CtIP proteins or fragments thereof Illustratively, the dimerization domain of a human CtIP protein may be represented by a polypeptide having the sequence SEQ ID NO. 4.

Similarly, the expression “tetramerization domain of a CtIP protein” refers to a continuous sequence of amino acids of a CtIP protein involved in the formation of dimers between two CtIP dimers or dimers of fragments thereof. Illustratively, the tetramerization domain of a human CtIP protein may be represented by a polypeptide having the sequence SEQ ID NO. 3.

In some embodiments, a dimerization domain and/or a tetramerization domain of a CtIP protein suitable for implementing the instant invention may be determined by the following method. Using the amino acid sequence of the N-terminal fragment of human CtIP, from aa 1 to aa 296, allows to identify similar sequence in CtIP protein from other species by sequence alignment software such as BLAST.

In some embodiment, the dimerization domain may comprise an amino acid sequence having at least 70% identities with the sequence SEQ ID NO. 4.

In some embodiment, the tetramerization domain may comprise an amino acid sequence having at least 70% identities with the sequence SEQ ID NO. 3.

Within the scope of the invention, at least 70% amino acid identity encompasses 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% amino acid identity.

The percentage of amino acid identity may be determined accordingly to the commonly methods used in the state of the art, in particular by performing a comparison of a given amino acid sequence with a reference amino acid sequence following optimal alignment.

The comparison of the sequence optimal alignment may be performed by using known algorithms. Most preferably, the amino acid identity percentage is determined using the CLUSTAL W software (version 1.82) the parameters being set as follows: (1) CPU MODE=ClustalW mp; (2) ALIGNMENT=“full”; (3) OUTPUT FORMAT=“aln w/numbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; (14) END GAPS=“default”; (15) GAP EXTENSION=“default”; (16) GAP DISTANCES=“default”; (17) TREE TYPE=“cladogram” and (18) TREE GRAP DISTANCES=“hide”.

In some embodiment, the dimerization domain may comprise an amino acid sequence having at least 85% amino acid identity, preferably 90% amino acid identity, with the sequence SEQ ID NO. 4.

In some embodiment, the tetramerization domain may comprise an amino acid sequence having at least 85% amino acid identity, preferably 90% amino acid identity, with the sequence SEQ ID NO. 3.

The position of the tetramerization domain and the dimerization domain of a CtIP protein with respect to the nuclease, in particular the Cas9 nuclease, may be indifferent within the fusion protein.

Illustratively, when ‘T’ represents the tetramerization domain and ‘D’ represents the dimerization domain of a CtIP protein, the fusion protein may be, from the N-terminal end to the C-terminal end, Cas9-T-D or Cas9-D-T, and is preferably Cas9-T-D.

In some embodiments, the fusion protein further comprises a domain of a CtIP protein comprising at least one cyclin-dependent kinase (CDK) phosphorylation site, preferably two CDK phosphorylation sites, more preferably three CDK phosphorylation sites.

The position of the tetramerization domain, the dimerization domain and the domain of a CtIP protein comprising at least one cyclin-dependent kinase (CDK) phosphorylation site with respect to the nuclease, in particular the Cas9 nuclease, may be indifferent within the fusion protein.

Illustratively, when ‘T’ represents the tetramerization domain, ‘D’ represents the dimerization domain and ‘P’ represents the domain of a CtIP protein comprising at least one cyclin-dependent kinase (CDK) phosphorylation site, the fusion protein may be, from the N-terminal end to the C-terminal end, as follows:
Cas9-T-D-P;
Cas9-D-T-P;
Cas9-T-P-D;
Cas9-D-P-T;
Cas9-P-T-D; or
Cas9-P-D-T.

In some embodiments, the fusion protein may be, from the N-terminal end to the C-terminal end, Cas9-T-D-P.

In some embodiments, the tetramerization domain and/or the dimerization domain and/or optionally the domain of a CtIP protein comprising at least one cyclin-dependent kinase (CDK) phosphorylation site may be localized within the amino acid sequence of the nuclease.

Illustratively, Oakes et al. have described hotspots within the Cas9 nuclease that tolerate domain(s) insertion(s) without affecting the Cas9 nuclease function, in particular DNA binding function and DNA cleavage function.

In certain embodiments, the domain of a CtIP protein comprising at least one cyclin-dependent kinase (CDK) phosphorylation site may comprise an amino acid sequence having at least 70% amino acid identity with SEQ ID NO. 14.

In some embodiments, the domain of a CtIP protein comprising at least one cyclin-dependent kinase (CDK) phosphorylation site may comprise an amino acid sequence having at least % identities, preferably 90% identities, with the sequence SEQ ID NO. 14.

The inventors observed that a mutation to replace a serine or a threonine amino acid, which is comprised within the CDK phosphorylation site, with a glutamic acid amino acid results in the mimicking of a phosphorylated state of the said phosphorylation site.

In certain embodiments, the at least one CDK phosphorylation site comprises a serine to glutamic acid (Ser/Glu) or a threonine to glutamic acid (Thr/Glu) substitution.

In certain embodiments, the fusion protein comprises a domain of a CtIP protein comprising two cyclin-dependent kinase (CDK) phosphorylation sites, each having a serine to glutamic acid (Ser/Glu) or a threonine to glutamic acid (Thr/Glu) substitution.

In some embodiments, the fusion protein comprises a domain of a CtIP protein comprising three cyclin-dependent kinase (CDK) phosphorylation sites, each having a serine to glutamic acid (Ser/Glu) or a threonine to glutamic acid (Thr/Glu) substitution.

In some embodiments, a dimerization domain of a CtIP protein, a tetramerization domain of a CtIP protein and one, two or three cyclin-dependent kinase (CDK) phosphorylation site may consist in the N-terminal domain of a CtIP protein.

In some embodiments, the fusion protein further comprises a nuclear localization domain.

Suitable classical or non-classical nuclear localization domain may be e.g. disclosed in Lange et al. (2007), Kosugi et al. (2009) and Marfori et al. (2011).

Illustratively, the nuclear localization domain may be the sequence PKKKRKV (SEQ ID NO. 17) of SV40, KRPAATKKAGQAKKKK (SEQ ID NO. 18) of nucleoplasmin, PAAKRVKLD (SEQ ID NO. 19) of c-Myc and MSRRRKANPTKLSENAKKLAKEVEN (SEQ ID NO. 20) of EGL-13.

In certain embodiments, the nuclear localization domain may be comprised in a sequence selected in a group comprising SEQ ID NO. 17, SEQ ID NO. 18, SEQ ID NO. 19 and SEQ ID NO. 20.

The nuclear localization domain may be located at any position within the fusion protein, i.e. at the N-terminus or the C-terminus of the fusion protein, (a) between (a-i) the Cas9 protein and (a-ii) the domains of the CtIP protein or (b) between two domains of the CtIP protein that are comprised in the fusion protein.

In certain embodiments, the nuclear localization domain is located within the fusion protein (a) between (a-i) the Cas9 protein and (a-ii) the domains of the CtIP protein, in particular (b) between (b-i) the Cas9 protein and (b-ii) the tetramerization domain of the CtIP protein comprised in the fusion protein described herein.

Due to a high conservation of CtIP proteins among eukaryotic species, CtIP may originate from any eukaryotic species, is in particular from an animal origin, and is more preferably of mammalian origin.

In certain embodiments, the CtIP protein is from human origin.

In certain embodiments, the Cas9 protein and the different domains of the CtIP protein may be spaced by one or more spacer peptides.

Indeed, the number of spacer amino acid sequences, when present in the fusion protein, and their location within the said fusion protein, may vary depending on the number of CtIP domains and on the ordering of the Cas9 protein and of the CtIP domains within the said fusion protein.

In some embodiments wherein the fusion protein comprises, from the N-terminal end to the C-terminal end, (i) a Cas9 protein, (ii) a Ct1P dimerization domain, (iii) a CtIP tetramerization domain and (iv) a polypeptide comprising one or more CDK-dependent phosphorylation sites, the said fusion protein may comprise:
a spacer amino acid sequence between the Cas9 protein and the CtIP dimerization domain, and/or
a spacer amino acid sequence between the CtIP dimerization domain and the CtIP tetramerization domain, and/or
a spacer amino acid sequence between the CtIP dimerization domain and the polypeptide comprising one or more CDK-dependent phosphorylation dependent sites.

Within the scope of the present invention, a “spacer” represents an amino acid sequence from 1 to 100 amino acid residues, which is inert, i.e. having no known biological activity, and intended to separate the domains from each other.

In other words, the spacer aims to reduce or inhibit the interaction(s) and/or interference(s) between the domains and to maintain their biological activities.

The expression “from 1 to 100 amino acid residues” encompasses 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99 amino acid residues.

In some embodiments, the spacer comprises less than 50 amino acid residues, preferably less than 25 amino acid residues.

In some embodiments, the tetramerization domain of a CtIP protein, the dimerization domain of a CtIP protein and optionally the domain of a CtIP protein comprising at least one cyclin-dependent kinase (CDK) phosphorylation site may originate from distinct species.

In certain embodiments, the tetramerization domain of a CtIP protein, the dimerization domain of a CtIP protein and optionally the domain of a CtIP protein comprising at least one cyclin-dependent kinase (CDK) phosphorylation site may originate from the same species.

In the latter embodiments, the tetramerization domain of a CtIP protein and the dimerization domain of a CtIP protein may originate from the same CtIP protein.

Illustratively, a protein comprising the dimerization domain of a CtIP protein and the tetramerization domain of a CtIP protein may be represented by an amino acid sequence having at least 70% amino acid identity with the sequence SEQ ID NO. 12.

In certain embodiments, a protein comprising the dimerization domain of a CtIP protein and the tetramerization domain of a CtIP protein may be represented by the amino acid sequence SEQ ID NO. 12.

In certain embodiments, the tetramerization domain of a CtIP protein, the dimerization domain of a CtIP protein and the domain of a CtIP protein comprising at least one cyclin-dependent kinase (CDK) phosphorylation site may originate from the same CtIP protein.

Illustratively, a protein comprising the dimerization domain of a CtIP protein, the tetramerization domain of a CtIP protein and the domain of a CtIP protein comprising at least one cyclin-dependent kinase (CDK) phosphorylation site may comprise, or alternatively may consist of, an amino acid sequence having at least 70% amino acid identity with a sequence selected in a group comprising SEQ ID NO. 2, SEQ ID NO. 6, SEQ ID NO. 7, SEQ ID NO. 8, SEQ ID NO. 9 and SEQ ID NO. 15, preferably SEQ ID NO. 6 and SEQ ID NO. 15.

In certain embodiments, the dimerization domain of a CtIP protein, the tetramerization domain of a CtIP protein and the domain of a CtIP protein comprising at least one cyclin-dependent kinase (CDK) phosphorylation site may be represented by an amino acid sequence selected in a group comprising SEQ ID NO. 2, SEQ ID NO. 6, SEQ ID NO. 7, SEQ ID NO. 8, SEQ ID NO. 9 and SEQ ID NO. 15, preferably SEQ ID NO. 6 and SEQ ID NO. 15.

In certain embodiments, the fusion protein may be represented by an amino acid sequence having at least 70% amino acid identity with a sequence selected in a group comprising SEQ ID NO. 21, SEQ ID NO. 22, SEQ ID NO. 23 and SEQ ID NO. 24.

In certain embodiments, the fusion protein may be represented by an amino acid sequence selected in a group comprising SEQ ID NO. 21, SEQ ID NO. 22, SEQ ID NO. 23 and SEQ ID NO. 24.

In certain embodiments the fusion protein may be represented by an amino acid sequence SEQ ID NO. 22, which refers to a fusion between the Cas9 nuclease and the HE domain of CtIP (1-296 aa), also referred as to “Cas9-HE” fusion.

A fusion protein according to the invention may be conventionally synthesized from a nucleic acid encoding the said fusion protein, by the mean of any technique of molecular biology known in the state of the art.

Alternatively, a fusion protein according to the invention may be produced by bioconjugation by the means covalent coupling between the nuclease and the domains of the CtIP protein.

Bioconjugation may be performed accordingly to the general principles and the methods described in Reddington and Howarth (2015), using the SpyTag/SpyCatcher technology; Shah and Muir (2014), using the intein's technology; Moll et al. (2001), using the leucine zipper technology.

### Nucleic Acids

The fusion protein may be provided through the in vitro or in vivo expression of a nucleic acid encoding said fusion protein.

In one aspect, the invention relates to a nucleic acid encoding a fusion protein as disclosed herein.

The nucleic acid encoding a fusion protein according to the invention comprises:
a nucleic acid sequence encoding a Cas9 protein, in particular a nucleic acid comprising a sequence having at least 70% nucleotide identity with the nucleic acid of sequence SEQ ID NO. 25;
a nucleic acid sequence encoding a tetramerization domain of a CtIP protein, in particular a nucleic acid comprising a sequence having at least 70% nucleotide identity with the nucleic acid of sequence SEQ ID NO. 43;
a nucleic acid sequence encoding a dimerization domain of a CtIP protein, in particular a nucleic acid comprising a sequence having at least 70% nucleotide identity with the nucleic acid of sequence SEQ ID NO. 44; and optionally
a nucleic acid sequence encoding a domain of a CtIP protein comprising at least one cyclin-dependent kinase (CDK) phosphorylation site, in particular a nucleic acid comprising a sequence having at least 70% nucleotide identity with the nucleic acid of sequence SEQ ID NO. 45.

In some embodiments, the nucleic acid encoding a tetramerization domain of a CtIP protein, the nucleic acid encoding a dimerization domain of a CtIP protein and the nucleic acid sequence encoding a domain of a CtIP protein comprising at least one cyclin-dependent kinase (CDK) phosphorylation site may comprise, or alternatively may consist of, a nucleic acid having at least 70% nucleotide identity with a nucleic acid sequence selected in a group comprising SEQ ID NO. 26, SEQ ID NO. 27 and SEQ ID NO. 28.

Within the scope of the invention, at least 70% nucleotide identity encompasses 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% nucleotide identity.

Percent nucleotide identity may be determined using the sequence comparison program NCBI-BLAST2 (Altschul et al., 1997). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, drop-off for final gapped alignment=25 and scoring matrix=BLOSUM62.

In some embodiments, the nucleic acids encoding the Cas9 protein, the tetramerization domain of a CtIP protein, the dimerization domain of a CtIP protein and the domain of a CtIP protein comprising at least one cyclin-dependent kinase (CDK) phosphorylation site may be separated by one or more nucleic acids encoding an amino acid spacer.

In some embodiments, the nucleic acid encoding a spacer is from 3 nucleotides to 300 nucleotides in length, preferably less than 150 nucleotides in length, more preferably less than 75 nucleotides in length.

In some embodiments, the nucleic acid encoding a fusion protein as described herein may comprise, or alternatively may consist of, a nucleic acid having at least 70% nucleotide identity with a sequence selected in a group of SEQ ID NO. 29, SEQ ID NO. 30 and SEQ ID NO. 31.

Another aspect of the invention relates to a nucleic acid vector for recombinant protein expression comprising a nucleic acid encoding a fusion protein as disclosed herein.

In some embodiments, the nucleic acid vector comprises a promoter, a terminator and optionally a regulating region in order to promote basal or controlled expression of the nucleic acid encoding the fusion protein according to the invention.

Within the scope of the present invention, the expression “basal expression” refers to a continuous expression of the nucleic acid encoding the fusion protein, irrespective of a defined time frame or a cellular context.

Within the scope of the present invention, the expression “controlled expression” refers to an expression that occurs within a defined time frame and/or within a defined cellular context.

For example, the nucleic acid vector may comprise regulating regions suitable to achieve expression in one given cellular type. Moreover, the nucleic acid vector may comprise regulating regions suitable to achieve expression during the presence of a given stimulus.

In some embodiments, suitable vectors may of viral origin, in particular selected in a group comprising an adenovirus, an adeno-associated virus (AAV), an alphavirus, a herpesvirus, a lentivirus, a non-integrative lentivirus, a retrovirus and a vaccinia virus.

### Delivery Particles

Another aspect of the invention further relates to a delivery particle comprising a fusion protein, a nucleic acid or a nucleic acid vector, as disclosed herein.

In certain embodiments, the delivery particle may be in the form of a lipoplexe, comprising cationic lipids; a lipid nano-emulsion; a solid lipid nanoparticle; a peptide based particle; a polymer based particle, in particular comprising natural and/or synthetic polymers.

In some embodiments, a polymer based particle may comprise a synthetic polymer, in particular, a polyethylene glycol (PEG), a polyethylene imine (PEI), a dendrimer, a poly (DL-Lactide) (PLA), a poly(DL-Lactide-co-glycoside) (PLGA), a polymethacrylate and a polyphosphoesters.

In some embodiments, the delivery may further comprise at its surface one or more targeting ligands suitable for specifically addressing said particle to a targeted cell.

In some embodiments, a polymer based particle may comprise a protein, in particular an antibody or a fragment thereof; a peptide; a mono-saccharide, an oligo-saccharide or a polysaccharide, in particular chitosan; a hormone; a vitamin; a ligand of a cellular receptor.

In some embodiments, the delivery particles according to the invention may be introduced in one or more target cells by the means of suitable methods known in the art, such as methods used for transfecting cells, which include electroporation, osmotic choc, sonoporation, cell squeezing and the like.

### Cells

In a still other aspect of the invention, one may consider a host cell comprising a fusion protein, a nucleic acid or a nucleic acid vector, as disclosed herein.

The host cell according to the invention may be indifferently a prokaryotic cell or a eukaryotic cell.

Illustratively, the host cell may be a yeast cell, a fungi cell, a plant cell or an animal cell.

In certain embodiments, an animal host cell according to the instant invention may encompass, without limitation, a cell of the central nervous system, an epithelial cell, a muscular cell, an embryonic cell, a germ cell, a stem cell, a progenitor cell, a hematopoietic stem cell, a hematopoietic progenitor cell, an induced Pluripotent Stem Cell (iPSC).

In some embodiments, the host cell may belong to a tissue selected in a group comprising a muscle tissue, a nervous tissue, a connective tissue, and an epithelial tissue.

In some embodiments, the host cell may belong to an organ selected in a group comprising a bladder, a bone, a brain, a breast, a central nervous system, a cervix, a colon, an endometrium, a kidney, a larynx, a liver, a lung, an oesophagus, an ovarian, a pancreas, a pleura, a prostate, a rectum, a retina, a salivary gland, a skin, a small intestine, a soft tissue, a stomach, a testis, a thyroid, an uterus, a vagina.

Without limitation the host cell may originate from a human or a non-human animal, in particular a dog, a cat, a mouse, a rat, a fly, a rabbit, a pig, a chicken, a mosquito, a zebrafish, a horse and a cow, or a plant in particular, rice, wheat, tomato, soya and corn.

In some embodiments, the host cell may be a microorganism, in particular selected in a group comprising bacteria and archaea.

### Pharmaceutical Composition

Another aspect of the invention relates to a pharmaceutical composition comprising (i) a fusion protein, a nucleic acid, a nucleic acid vector or a delivery particle as disclosed herein, and (ii) a pharmaceutically acceptable vehicle.

The formulations of pharmaceutical compositions suitable to implement the disclosed invention may be obtained by following the routine and commons methods and principles in the art.

In some embodiments, a suitable pharmaceutically acceptable vehicle according to the invention may include any conventional solvents, dispersion media, fillers, solid carriers, aqueous solutions, coatings, antibacterial and antifungal agents, isotonic and absorption delaying agents, and the like.

In certain embodiments, suitable pharmaceutically acceptable vehicles may include, water, saline, phosphate buffered saline, dextrose, glycerol, ethanol and a mixture thereof.

In some embodiments, pharmaceutically acceptable vehicles may further comprise minor amounts of auxiliary substances such as wetting or emulsifying agents, preservatives or buffers, which enhance the shelf life or effectiveness of the cells.

Except insofar as any conventional media or agent is incompatible with the active ingredient, use thereof in the pharmaceutical compositions of the present invention is contemplated.

### Uses

Another aspect of the invention relates to a fusion protein, a nucleic acid, a nucleic acid vector or a delivery particle, as disclosed herein, for use as a medicament.

In some embodiments, the fusion proteins, the nucleic acids, the nucleic acid vectors or the delivery particles, as disclosed herein, may be for use for the preparation of a medicament, in particular a medicament intended to treat a disorder by genic therapy.

The said disorder may be selected in a group comprising a genetic disorder, a cancer, an infectious disease and a neurodegenerative disease.

In some embodiments, the genetic disorder may be selected in the non-limitative group comprising Achondroplasia, Alpha-1 Antitrypsin Deficiency, Antiphospho lipid Syndrome, Autism, Autosomal Dominant Polycystic Kidney Disease, Breast cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Fanconi Anemia, Factor V Leiden Thrombophilia, Familial Hypercholesterolemia, Familial Mediterranean Fever, Fragile X Syndrome, Gaucher Disease, Hemochromatosis, Hartnup's Disease, Haemophilia, Holoprosencephaly, Huntington's disease, Kartagener's Syndrome, Klinefelter syndrome, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Tay-Sachs, Thalassemia, Trimethylaminuria, Tuberous Sclerosis, Turner Syndrome, Velocardiofacial Syndrome, WAGR Syndrome and Wilson Disease.

In some embodiments, the cancer is selected in a non-limitative group comprising a bladder cancer, a bone cancer, a brain cancer, a breast cancer, a cancer of the central nervous system, a cancer of the cervix, a cancer of the upper aero digestive tract, a colorectal cancer, an endometrial cancer, a germ cell cancer, a glioblastoma, a Hodgkin lymphoma, a kidney cancer, a laryngeal cancer, a leukaemia, a liver cancer, a lung cancer, a myeloma, a nephroblastoma (Wilms tumor), a neuroblastoma, a non-Hodgkin lymphoma, an oesophageal cancer, an osteosarcoma, an ovarian cancer, a pancreatic cancer, a pleural cancer, a prostate cancer, a retinoblastoma, a skin cancer (including a melanoma), a small intestine cancer, a soft tissue sarcoma, a stomach cancer, a testicular cancer and a thyroid cancer.

In some embodiments, the infectious disease may be selected in the non-limitative group comprising Acute rheumatic fever, Anthrax, Australian bat lyssavirus,

Avian influenza (Bird Flu), Babesiosis, Barmah Forest virus, Botulism, Brucellosis, Campylobacteriosis, Chancroid, Chickenpox, Chikungunya, Chlamydia, Cholera, Creutzfeldt-Jakob disease (CJD), Cryptosporidiosis, Cytomegalovirus (CMV), Dengue, Dientamoeba fragilis, Diphtheria, Donovanosis, Ebola virus disease, Epidemic keratoconjunctivitis, Epstein-Barr virus (EBV), Fifth disease, Gastroenteritis, German measle (Rubella), Giardiasis, Gonorrhoea, Glandular fever (Infectious mononucleosis), Haemolytic uraemic syndrome, Haemophilus influenzae Type b (Hib), Hand foot and mouth disease, Hendra virus, A/B/C/D/E Hepatitis, Human immunodeficiency virus (HIV), Influenza, Japanese encephalitis, Kunjin virus, Legionnaires' disease, Leprosy, Leptospirosis, Listeriosis, Lyme disease, Lymphogranuloma venereum (LGV), Malaria, Maternal sepsis (Puerperal fever), Measles, Meningococcal disease, MERS coronavirus, MRSA , Mumps, Murray Valley encephalitis (MVE), Norovirus, Pandemic influenza, Parvovirus B19, Pertussis, Plague, Pneumococcal disease, Poliomyelitis, Psittacosis, Q fever, Rabies, Rat Lung worm, Respiratory syncytial virus (RSV), Rheumatic heart disease, Rickettsia, Ross River virus, Rotavirus, Rubella, Salmonellosis, SARS coronavirus, Shiga toxigenic E. Coli (STEC/VTEC), Shigellosis, Shingles, Smallpox, Syphilis, Tetanus (lock-jaw), Tuberculosis (TB), Tularemia, Typhoid, Typhus, Varicella-Zoster virus, Viral haemorrhagic fevers, Whooping cough, Yellow fever and Zika virus.

In some embodiments, the neurodegenerative disease may be selected in the non-limitative group comprising Alzheimer's disease, Amyotrophic lateral sclerosis, Down's syndrome, Friedreich's ataxia, Huntington's disease, Lewy body disease, Parkinson's disease and Spinal muscular atrophy.

In another aspect, the invention also relate to a pharmaceutical composition according to the description herein for use as an active agent for editing the genome into at least one target cell.

In some embodiments, the fusion proteins, the nucleic acids, the nucleic acid vectors, the delivery particles or the pharmaceutical compositions, as disclosed herein, may be administered to an individual in need thereof by any route, i.e. by an oral administration, a topical administration or a parenteral administration, e.g., by injection, including a sub-cutaneous administration, a venous administration, an arterial administration, in intra-muscular administration, an intra-ocular administration and an intra-auricular administration.

In certain embodiments, the administration of the fusion proteins, the nucleic acids, the nucleic acid vectors, the delivery particles or the pharmaceutical compositions, as disclosed herein, by injection may be directly performed in the target tissue of interest, in particular in order to avoid spreading of the said product.

Other suitable modes of administration may also employ pulmonary formulations, suppositories, and transdermal applications.

In some embodiments, an oral formulation according to the invention includes usual excipients, such as, for example, pharmaceutical grades of mannitol, lactose, starch, magnesium stearate, sodium saccharine, cellulose, magnesium carbonate, and the like.

In some embodiments, an effective amount of said compound is administered to said individual in need thereof.

Within the scope of the instant invention, an “effective amount” refers to the amount of said compound that alone stimulates the desired outcome, i.e. alleviates or eradicates the symptoms of the encompassed a genetic disorder.

It is within the routine and the common knowledge of a skilled artisan to determine the effective amount of fusion proteins, the nucleic acids, the nucleic acid vectors, the delivery particles or the pharmaceutical compositions, as disclosed herein, in order to observe the desired outcome.

Within the scope of the instant invention, the effective amount of the product to be administered may be determined by a physician or an authorized person skilled in the art and can be suitably adapted within the time course of the treatment.

In certain embodiments, the effective amount to be administered may depend upon a variety of parameters, including the material selected for administration, whether the administration is in single or multiple doses, and the individual's parameters including age, physical conditions, size, weight, gender, and the severity of the disease to be treated.

In certain embodiments, an effective amount of the fusion protein or the delivery particle may comprise from about 0.001 mg to about 3000 mg, per dosage unit, preferably from about 0.05 mg to about 100 mg, per dosage unit.

Within the scope of the instant invention, from about 0.001 mg to about 3000 mg includes, from about 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg and 2950 mg, per dosage unit.

In certain embodiments, the of the fusion protein or the delivery particle may be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day.

In other embodiments, an effective amount of the nucleic acid encoding the fusion protein or the nucleic acid vector may comprise from about 1 ng to about 1 mg, per dosage unit, preferably from about 50 ng to about 100 μg, per dosage unit.

Within the scope of the instant invention, from about 1 ng to about 1 mg includes, about 2 ng, 3 ng, 4 ng, 5 ng, 6 ng, 7 ng, 8 ng, 9 ng, 10 ng, 20 ng, 30 ng, 40 ng, 50 ng, 60 ng, 70 ng, 80 ng, 90 ng, 100 ng, 150 ng, 200 ng, 250 ng, 300 ng, 350 ng, 400 ng, 450 ng, 500 ng, 550 ng, 600 ng, 650 ng, 700 ng, 750 ng, 800 ng, 850 ng, 900 ng, 950 ng, 1 μg, 2 μg, 3 μg, 4 μg, 5 μg, 6 μg, 7 μg, 8 μg, 9 μg, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg and 950 μg per dosage unit.

In certain embodiments, the nucleic acid encoding the fusion protein or the nucleic acid vector may be administered at dosage levels sufficient to deliver from about 0.01 ng/kg to about 10 μg/kg, from about 0.1 ng/kg to about 5 μg/kg, preferably from about 1 ng/kg to about 1 μg/kg of subject body weight per day.

### Methods

The methods disclosed herein may be achieved in vitro, in vivo or ex vivo.

The present invention also relates to a method for editing a genome into at least one target cell comprising at least the step of administering to an individual in need thereof of a fusion protein, a nucleic acid, a nucleic acid vector, a delivery particle, as disclosed herein.

Another aspect of the invention relates to a method for editing a genome into at least one target cell comprising at least the step of administering to an individual in need thereof a pharmaceutical composition as disclosed herein.

As mentioned above, the genome editing may be performed in a target cell, irrespective of its origin, i.e. in a prokaryote target cell or a eukaryote target cell.

The present invention also relates to a method for treating a genetic disorder, a cancer and/or an infectious disease comprising at least the step of administering to an individual in need thereof of a fusion protein, a nucleic acid, a nucleic acid vector, a delivery particle or a pharmaceutical composition, as disclosed herein.

### Kits

In another aspect, the invention relates to a kit for editing the genome of at least a target cell, comprising:
a fusion protein as described herein, a nucleic acid encoding the said fusion protein, a nucleic acid vector comprising the said nucleic acid or a delivery particle comprising the said fusion protein, the said nucleic acid or the said nucleic acid vector, as disclosed herein; and
one or more site-specific guide RNAs (gRNAs) or a nucleic acid vector for expressing one or more site specific guide RNAs (gRNAs).

It is needless to mention that the kit disclosed herein may be also of use for treating and/or preventing a cancer and/or an infectious disease.

Specific guide RNAs may be designed according to the common rules and principles disclosed in the state in the art, in particular Hsu et al. (2013), Mali et al. (2013), Koferle et al. (2016), WO2015153940, WO2016196805, WO2016183402.

Alternatively, guide RNAs may be designed by using algorithms available online from commercial sources such as Benchling®, Desktop genetics® or from academic sources such as the Zhang laboratory of the Massachusetts Institute of Technology (MIT, crispr.mit.edu), the French research network TEFOR (crispor.org), and many others.

### EXAMPLES

### Example 1—Materials and Methods

### 1.1 Plasmid Construction

Guide RNA sequences were cloned in MLM3636 derived vector (Addgene #43860) and Cas9-expression vector (Addgene #41815) was used. CtIP-expression vector was kindly sent by Xiao Wu lab (UCSC : chr18:22,936,852-23,026,240) (Wang et al., 2013). CtIP fragments were amplified by PCR and inserted between EcoRI and Agel restriction sites in Cas9-expression vector by standard cloning. GFP donor plasmid, containing a GFP transgene with an artificial splice acceptor site, E2A-GFP coding sequence and bGH polyA sequence flanked by 800 bp homology arms to the AAVS1 locus, was as described by de Kelver et al. (2010). Guide RNAs and donor plasmids targeting the human ATF4, GABP, TGIF2, RAD21, CREB genes were from the Mendenhall lab (Addgene #72350, #72351, #64253 and #64254).

### 1.2 Cell Culture and Transfections

Cells were all cultured at 37° C. in a humidified chamber with 5% C0₂ and transfected with the AMAXA electroporation system. HEK293 cells were cultured in DMEM supplemented with 10% fetal bovine serum (FBS). 10⁶ cells were transfected with 1 μg of Cas9 expression plasmid, 1 μg of gRNA expression plasmid and 1 μg of p84 donor using
V solution and A-023 program. RG37DR cells were cultured in DMEM supplemented with 10% FBS and transfected with 1 μg of Cas9 expression plasmid, 1 μg of gRNA expression plasmid and 1 μg of p84 donor using NHDF solution and P-022 program. HCT116 cells were cultured in McCoy supplemented with 10% FBS and transfected with 4 μg of Cas9 expression plasmid, 2 μg of gRNA expression plasmid and 6 μg of p84 donor using V solution and D-032 program. Electroporations were performed according to the manufacturer's instructions. Lonza 4D-Nucleofector™ System; P3 Primary Cell 4D-Nucleofector® X, program: CM-113.

### 1.3 Analysis of HDI by FACS

When targeting the AAVS1 locus with the p84 donor, targeted integration of GFP cDNA results in cells becoming GFP-positive, which can be easily monitored by FACS analysis. Cells were analyzed for GFP expression by flow cytometry using an Accuri C6 analyzer (BD BIOSCIENCES®) 6 to 7 days after transfection. Relative HDI rate was calculated by normalizing HDI rates by the GDTI rate induced by TALEN alone or Cas9.

### 1.4 Analysis of Imprecise-Mutation Rates by the T7EI Assay

T7 Endonuclease I (T7EI) assays were performed to analyze the rates of imprecise mutations induced by End Joining DNA DSB repair pathways as previously described (Piganeau et al., 2013) using the following primers: T7AAVFw cagcaccaggatcagtgaaa

(SEQ ID NO. 32) and T7AAVRev ctatgtccacttcaggacagca (SEQ ID NO. 33). Sequence modification frequencies were estimated as previously described in Renaud et al., 2016, by the mean of the following formula:

% indels=1−(1−Xc)^{1/2}

wherein Xc represents the rate of cleaved products; if Xc<0.15, % indels=Xc/2. Relative mutation rates were calculated by normalizing mutation rates by the mutation rate induced by Cas9.

### 1.5 Analysis of Construction Expression Levels by Western Blot

Proteins were isolated 48 h after transfection. Cells were resuspended in lysis buffer (Tris-HCl 50 mM pH7, NaCl 150 mM, Triton X100 1%, SDS 0.1%, EDTA 1 mM, DTT 1 mM, aprotinine 1 μg/μL, pepstatine 10 μg/μL, leupeptine 1 μg/μL), centrifuged at 13,000 rpm and 4° C. for 15 min and supernatants were used. Western blots were performed by standard Tris-glycine SDS-PAGE followed by transfer to nitrocellulose membranes. Following blocking with 5% BSA in TBS-T (Tris 0.024 M, NaCl 0.137 M, KCl 2.68 mM and Tween 20 0.1%), membrane were probed with anti-Cas9 (Novus Biologicals, NBP2-36440SS) at lug/mL and anti-tubulin (Sigma, T6074200UL) at 0.1 μg/mL and visualized by chemiluminescence.

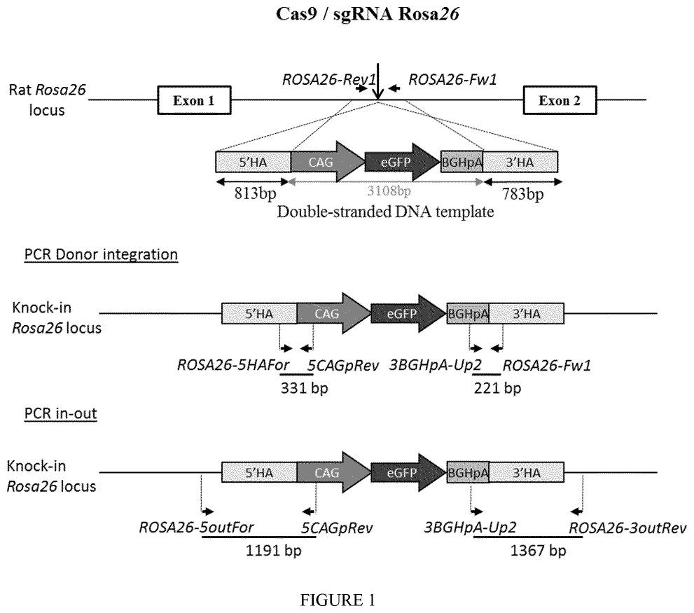

### 1.6 Generation of Genome Edited Rats (FIG. 1)

Zygotes were obtained from super-ovulated Sprague-Dawley rats (Charles River, l'Arbresle, France) and microinjected as previously described in detail (Remy et al., 2014). Briefly, linearized excised donor DNA was composed of the CAG promoter controlling GFP expression flanked by homology arms of 800 bp of Rosa26 contiguous to the site of cleavage recognized by a sgRNA (Menoret et al., 2015) (SEQ ID NO. 47). The Cas9-HE or Cas9 mRNAs, sgRNA and donor DNA were mixed (50, 10 and 2 ng/μl, respectively) and microinjected into the pro-nucleus and cytoplasm of the zygotes. Zygotes surviving microinjection were implanted into pseudo-pregnant females. At day 14, females were sacrificed and DNA was extracted from embryos for genotyping. Genotyping was performed using the primers and PCRs conditions described below and a hetero-duplex mobility shift assay using microfluidic capillary electrophoresis previously described (Chenouard et al., 2016) as well as sequencing of amplicons.

### Primers and PCR Conditions for Donor Integration:

rROSA-5HAFor:
(SEQ ID NO. 34)
TTCTTCCACTTGCGATCCTTG
5CAGpRev:
(SEQ ID NO. 35)
GGCTATGAACTAATGACCCCGTAAT
3BGHpA-Up2:
(SEQ ID NO. 36)
CCAGATTTTTCCTCCTCTCCTG
rROSAfw1:
(SEQ ID NO. 37)
TGAACTGTGAATAGGCCCAAGTG

### Program:

5 min of 95° C.

35 cycles of (i) 10 sec at 95° C., (ii) 10 sec at 60° C., (iii) 30 sec at 72° C.

3 min at 72° C.

4° C.

### Primers and PCR Conditions for Donor In-Out:

rROSA26-5outFor:
(SEQ ID NO. 38)
TCCCACCCTCCCCTTCCTCT
5CAGpRev:
(SEQ ID NO. 39)
GGCTATGAACTAATGACCCCGTAAT
3BGHpA-Up2:
(SEQ ID NO. 40)
CCAGATTTTTCCTCCTCTCCTG
rROSA26-3outRev:
(SEQ ID NO. 41)
TGGGTATCACTGGCTGTCCTAGATA

### Program:

5 min of 95° C.

35 cycles of (i) 30 sec at 95° C., (ii) 30 sec at 62° C., (iii) 2 min at 72° C.

3 min at 72° C.

4° C.

### Primers and PCR Conditions for NHEJ:

rROSAfw1:
(SEQ ID NO. 37)
TGAACTGTGAATAGGCCCAAGTG
rROSArev1:
(SEQ ID NO. 42)
GCATTTTAAAAGAGCCCAGTACTTCA

### Program:

5 min à 95° C.

35 cycles of (i) 10 sec at 95° C., (ii) 10 sec at 60° C., (iii) 30 sec at 72° C.

3 min at 72° C.

4° C.

### 1.7 Immunocytochemistry

Briefly, cells were fixed with PBS containing 8% paraformaldehyde for 20 min at 4° C. After washing with PBS, they were permeabilized and blocked with 0.1% TritonX-100 for 15 min at 4° C. After washing with PBS, the cells were blocked with 1% BSA and 10% Horse serum for 1 hour at room temperature. Then the cells were incubated, with anti-Human TRA-1-60 antibody conjugated to Alexa Fluor 488 (d: 1/10; BD PHARMINGEN®) and with anti-Human OCT3/4 antibody (d:1/40; R&D Systems), overnight at 4° C. in the dark. For the OCT3/4 staining, the cells were incubated the next day with a donkey anti-goat antibody conjugated to Alexa Fluor 555 (d: 1/1000; LIFE TECHNOLOGIES®) for 1 hour at room temperature in the dark. Counterstaining was performed using Hoechst (d:1/4000; INVITROGEN®) for 10 min at room temperature. The stained cells were analyzed by a Nikon Eclipse Ti microscope.

### 1.8 Analysis of Indel Mutation Patterns

DNA was isolated from transfected cells (EZNA tissue DNA kit, OMEGA BIOTECK®) and the target region amplified by PCR with Phusion Polymerase (NEB®). Each sample was assigned to a primer set with a unique barcode to enable multiplex sequencing. PCR products were purified on a 2% agarose gel and treated by the MNHN genomics center and sequences on Ion Torrent PGM. A custom python pipeline was used to count and characterize indels as detailed in Renaud et al. (2016). All sequence data from Tables 2 and 3 are available from NCBI BioPRoject with the accession number PRJNA433647.

### 1.9 RPA Foci Formation Assay

24 hours after plating, RG37 fibroblast cells were transfected with siRNA using Interferin (Polyplus, OZYME®). siNT(control): AUGAACGUGAAUUGCUCAA(dTdT) (SEQ ID NO. 76). siCtIP: GCUAAAACAGGAACGAAUC (SEQ ID NO. 77). 3 days after plating, cells were transfected with expression plasmids for Cas9, Cas9-HE, Cas9-CtIP using JetPei (Polyplus, OZYME®). 5 days after plating cells were X-rays irradiated at 6 Gy (XRAD 320, 1.03 Gy/min). At 0, 1, 2, 4, 6 and 8 h after irradiation, cells on coverslips were pre-permeabilized with PBS-Triton 0.25% for 3 min. on ice, then fixed in paraformaldehyde 2% for 15 min. The cells were then incubated with PBS containing 0.5% Triton X-100 for 5 min at room temperature for permeabilization.
After blocking in PBS containing 3% BSA and 0.05% Tween-20 solution for 30 min. at room temperature, immunostaining was performed using the following primary antibody: mouse anti-RPA (1:300, ANA19L, MILLIPORE®). Incubation was performed for 1 h30 at 37° C. with antibody diluted in PBS containing 3% BSA and 0.05% Tween-20. Next, the coverslips were incubated for 45 min. with Alexa 488-conjugated anti-mouse secondary antibody (LIFE TECHNOLOGIES®) at 37° C. and mounted in mounting medium (DAKO®) supplemented with 40,60-diamidino-2-phenylindole (DAPI) (SIGMA®). Images were captured using a ZEISS® Axio Imager Z1 microscope with a 63× objective equipped with a HAMAMATSU® camera. Acquisition was performed using AxioVision (4.7.2.). Images were imported, processed and merged in the ImageJ software.

### 1.10 Statistical Tests

Nonparametric Mann-Whitney t-tests were performed to determine significant differences in efficacy betweenCas9-CtIP fusion and derivatives thereof, on one hand, and Cas9 nucleases (*, P <0.05; **, P<0.005; ***, P<0.0005; ****, p<0.0001). Error bars indicate standard deviation.

### Example 2—CtIP Recruitment at the Cleavage Site Stimulates HDI of GFP cDNA at the AAVS1 Safe Harbor Locus

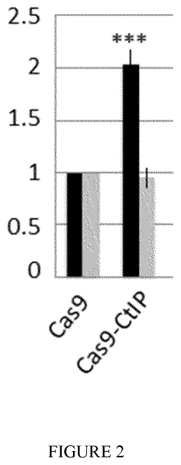

In order to improve the HDI rate, CtIP protein has been recruited at the target locus were tested. CtIP is a protein directly involved in early steps of HR repair by triggering end resection with the Mre11/Rad50/Nbs1 complex (MRN) (Komatsu, 2016; Liu and Huang, 2016). A well-established model system was used herein, consisting in the targeted insertion of a GFP cDNA at the AAVS1 safe harbor locus, which locus is of high interest for gene therapy and for experiments requiring robust transgene expression from modified cells.
RG37DR immortalized human fibroblasts were transfected with CtIP fused to Cas9, and a guide RNA (gRNA) designed to target Cas9-CtIP binding at the site of the DSB.
The gRNA sequence is the following:
GGGGCCACTAGGGACAGGATgttttagagctaGAAAtagcaagttaaaataaggctagtccgttatcaacttg aaaaagtggcaccgagtcggtgc (SEQ ID NO. 46), in which UPPERCASEs correspond to the AAVS1 target specific sequence and LOWERCASEs correspond to the guide RNA scaffold.
This allowed stimulating insertion of the GFP donor by 2 fold, as compared to Cas9 alone (FIG. 2). The imprecise-mutation rate (% indels), as measured by the T7EI assay, was not significantly modified when using Cas9-Ctlp compared to Cas9 (FIG. 2).
Altogether, these results show that CtIP recruitment at the nuclease cut site, through a fusion to Cas9, can improve homology-directed integration of an exogenous donor without modifying the imprecise mutation rate.

### Example 3—Recruitment of the N-Terminal Fragment Spanning aa 1 to 196 of CtIP is Sufficient to Improve HDI of GFP cDNA at the AAVS1 Locus

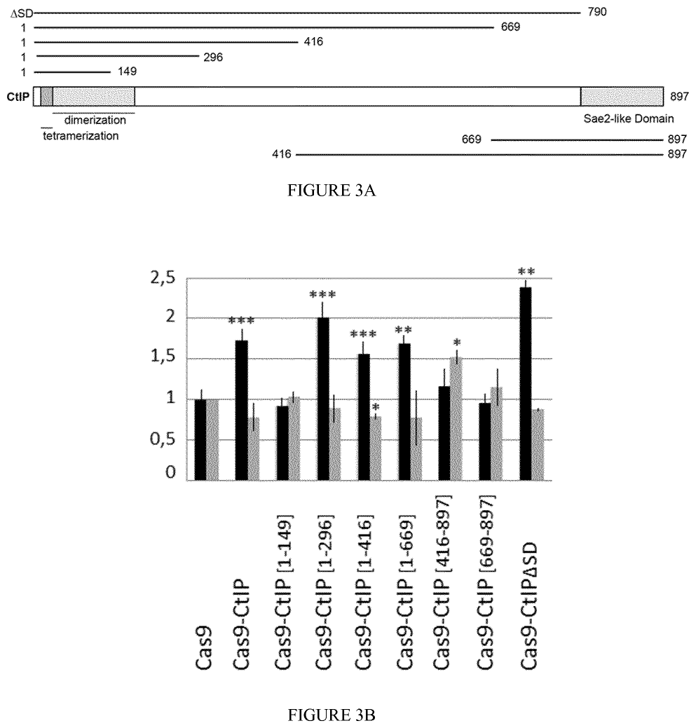

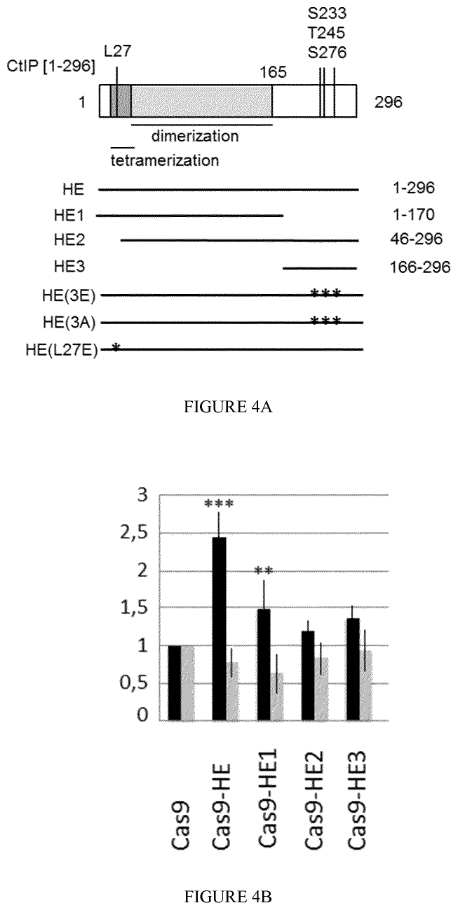

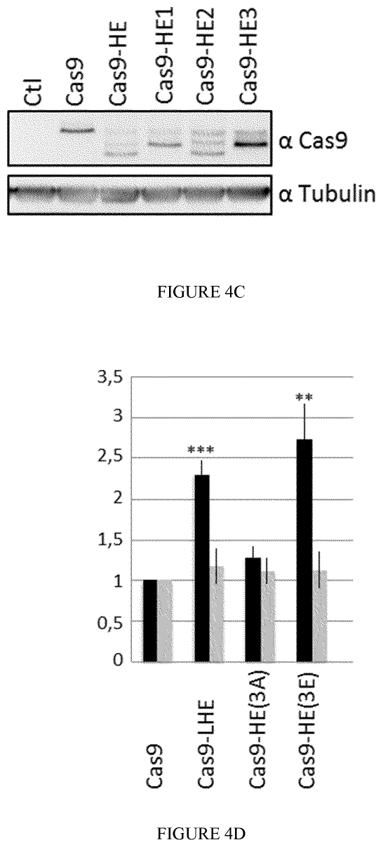

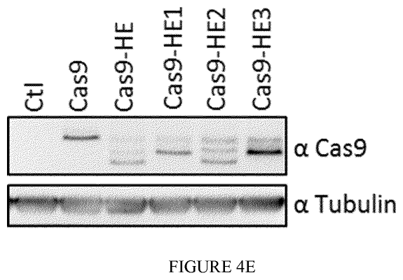

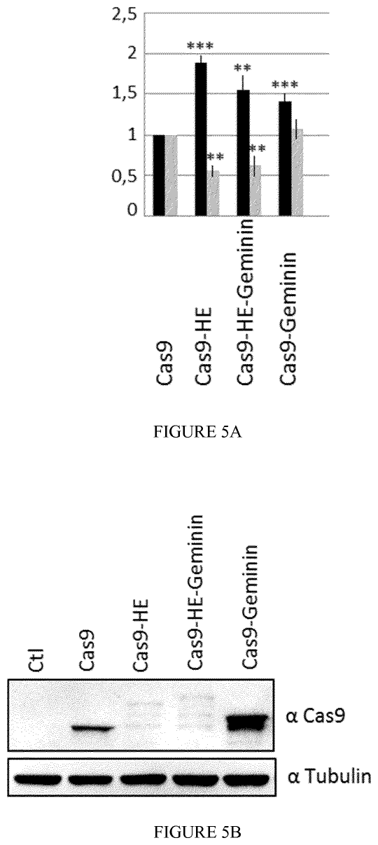

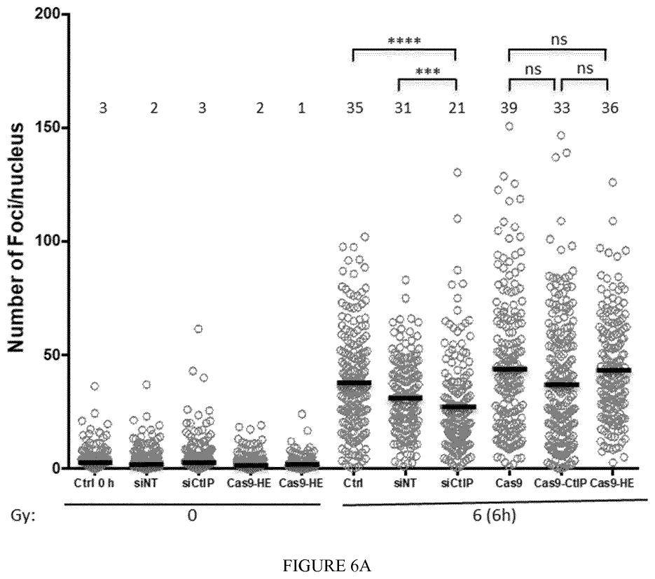

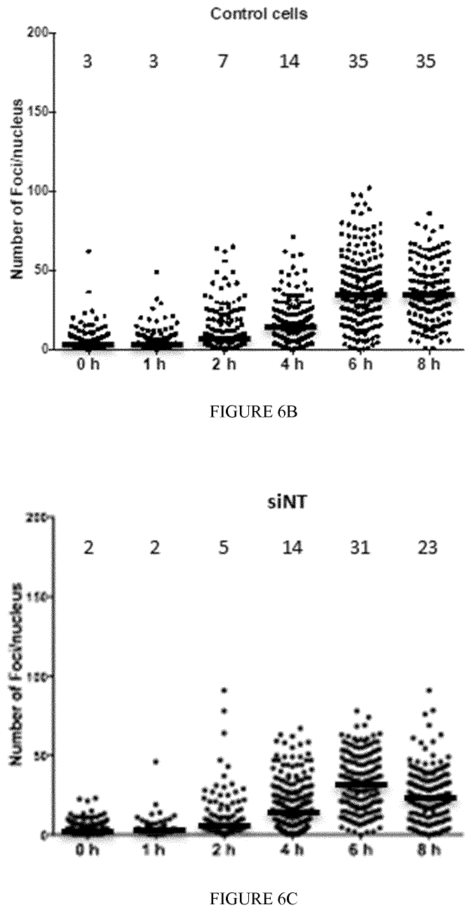

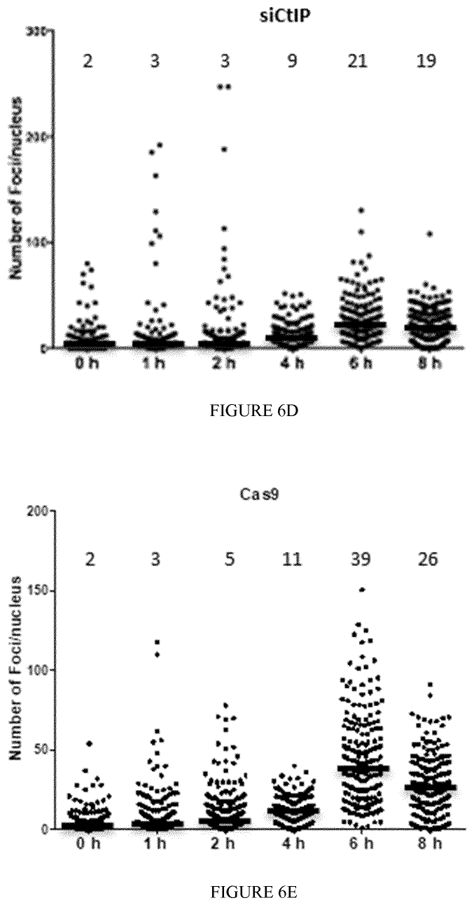

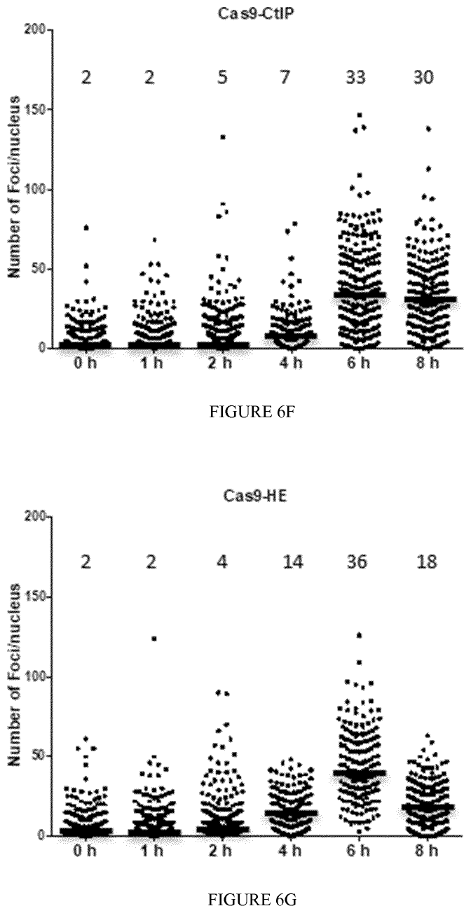

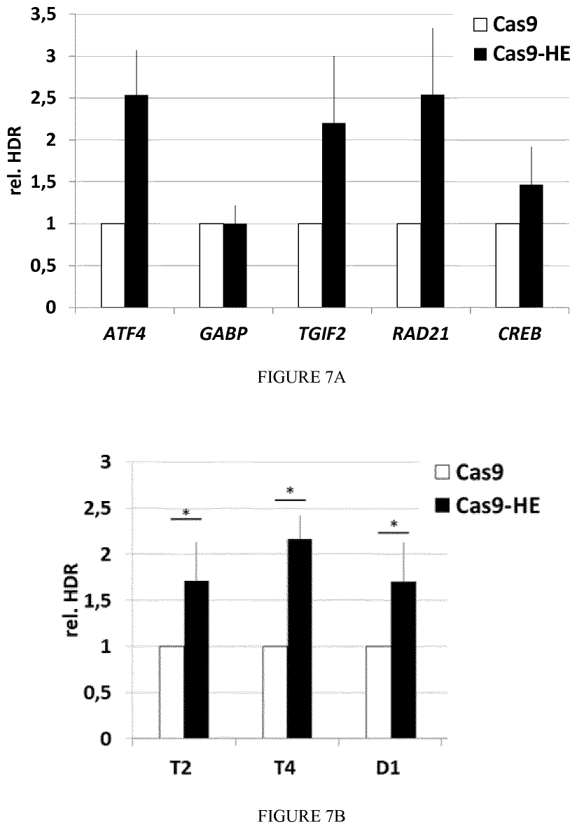

In order to examine how CtIP recruitment at the cut site can improve the homology-dependent insertion of an exogenous donor, CtIP was systematically truncated. Series of CtIP deletions, progressively removing approximately 200 amino acids from N- or C-terminal ends were tested (FIG. 3A).
Truncated CtIP proteins are as follows:

1-149: SEQ ID NO. 5

1-296 (HE): SEQ ID NO. 6

1-416: SEQ ID NO. 7

1-669: SEQ ID NO. 8

1-790 (deltaSD): SEQ ID NO. 9

416-897: SEQ ID NO. 10

669-897: SEQ ID NO. 11.

Truncated CtIP proteins were fused to Cas9 nuclease and tested in RG37DR cells on AAVS1 locus using the gRNA of sequence SEQ ID NO. 46 (see above).
When C-terminal deletions were tested, it was observed that deleting from aa296 to the C-terminal end of CtIP did not affect HDI stimulation and that the L2 fragment from the aa 1 to 296 was sufficient to stimulate HDI as efficiently as full-length CtIP (FIG. 3B).
Conversely, when testing N-terminal deletions, it was observed that the L2 fragment was sufficient for HDI stimulation and that all further N-terminal deletions were unable to stimulate HDI (FIG. 3B), despite being expressed at similar or apparently higher levels, as measured by western blot (not shown), and inducing roughly similar levels of imprecise mutations, as measured by the T7EI assay (FIG. 3B).
It emerges from this data that the N-terminal part of CtIP (1-296 aa; SEQ ID NO. 6) is sufficient for HDI stimulation by CtIP without modifying the imprecise mutation rate. The N-terminal fragment (1-296) was coined “HE” for “Homogy-dependent transgene integration enhancer domain”.

### Example 4—The CDK Phosphorylation Sites and CtIP Tetramerization Domain are Important for HDI Stimulation

In order to clarify how the small HE domain of CtIP stimulates homology-directed insertion of donor DNA, different HE mutants at AAVS1 locus in HEK293 cells were tested. HEK293 cells were used, rather than RG37DR cells, to facilitate detection of nuclease fusion proteins by western blot.
First, three HE fragments were engineered, (1) HE1 (1-170 aa; SEQ ID NO. 12) lacking 3 sites that are phosphorylated by CDK in CtIP and known to be necessary for its activity in HR (Wang et al., 2013), (2) HE2 (46-296 aa; SEQ ID NO. 13) lacking the first 45 aa which block CtIP/MRN interaction and CtIP tetramerization (Davies et al., 2015) and (3) HE3 (166-296 aa; SEQ ID NO. 14) containing the 3 CDK phosphorylation sites (FIG. 4A).
From the three HE fragments tested, HE1 was the only fragment shown to significantly stimulate homology-directed insertion of the GFP donor, although not as efficiently as the complete HE (FIG. 4B).
Because the HE domain contains 3 CDK sites, it was determined whether these phosphorylation sites are required for the effect of HE on Cas9 activity. For that purpose, these 3 sites were mutated either to alanine, HE(3A) (SEQ ID NO. 16), to block phosphorylation, or to glutamic acid, HE(3E) (SEQ ID NO. 15), to mimic phosphorylation by CDK (FIG. 4A).
The Cas9-HE(3E) mutant (SEQ ID NO. 24) led to HDI of GFP cDNA comparable to those achieved with Cas9-HE (SEQ ID NO. 22) (FIG. 4D).
In contrast, when using the Cas9-HE(3A) mutant, in which CDK phosphorylation is not possible, HDI levels were similar to those achieved with Cas9, showing that these sites are essential for improving HDI with the CtIP HE domain (FIG. 4D).

### Example 5—Cas9-HE is More Efficient than Cas9-Geminin at Stimulating HDI

As mentioned above, Cas9 fused to the first 110 aa of Geminin can improve homology-directed integration (Gutschner et al., 2016). In order to compare Cas9-HE and Cas9-geminin fusions, both fusions were assayed for their capacities of stimulating HDI at the AAVS1 locus in HEK293 cells.
As expected, the results obtained with Cas9-Geminin were in agreement with to those reported by Gutschner et al. (FIG. 5A). However, Cas9-HE was more efficient than Cas9-Geminin in increasing the frequency of HDI (FIG. 5B).

### Example 6—Cas9-HE Results in More Efficient HDI in Rat Oocytes

The efficiency of HDI for the generation of genome edited rats using Cas9-HE or Cas9 were compared. To this end, (1) a long donor DNA (4.7 kb), (2) sgRNAs targeting the Rosa26 locus and (3) Cas9-HE or Cas9 mRNA, were co-microinjected into rat zygotes. Table 1 below indicates the measured parameters.

TABLE 1
Comparison of Cas9-HE vs Cas9 to obtain homology-directed
transgene integration into the rat Rosa26 locus.

| | Eggs | | E14 | | | Random |
|---|---|---|---|---|---|---|
| Cas9 | injected | Eggs | embryos | Indels¹ | HR² | Transgenic³ |
| form | (% survival) | transfered | (% transfered) | (% E14) | (% E14) | (% E14) |
| Cas9-HE | 216 (75.0) | 154 | 37 (24.0) | 29 (78.3) | 3 (8.1) | 2 (5.4) |
| Cas9 | 284 (77.8) | 211 | 84 (39.8) | 62 (73.8) | 1 (1.2) | 1 (1.2) |

¹Indels generated by NHEJ, defined by sequencing of PCR amplicons performed with primers rROSAfw1 and rROSArev1 in embryos in which the 2 PCRs for donor integration were negative;
²HR, homologous recombination defined as by sequencing of positive of both PCRs in-out and positive of both PCRs for donor integration;
³Random transgenic defined as PCRs in-out negative and both PCRs for donor integration positive.

As indicated in Table 1 above, zygotes that survived to microinjection were re-implanted in foster mothers and embryos at day 14 of gestation, were harvested (with higher frequencies in Cas9 microinjected zygotes −24% and 39.8% for Cas9-HE and Cas9, respectively) and genotyped using the strategy depicted in FIG. 1.
Sequencing of PCR amplicons spanning the targeted sequence revealed similar frequencies of indels due to NHEJ in both conditions (78.3% and 73.8% for Cas9-HE and Cas9, respectively). Importantly, integration by HR was increased in zygotes microinjected with Cas9-HE—representing 8.1% and 1.2% of harvested embryos for Cas9-HE and Cas9, respectively). Thus, Cas9-HE increased the frequency of integration by HR compared to Cas9 without increasing its cleavage activity since NHEJ frequencies were comparable. One potential concern with overexpression of Cas9-HE is that it might interfere with endogenous CtIP activity. In order to examine this possibility, a RPA foci formation assay was performed. After resection mediated by CtIP during DSB repair by HR, 3′ single strand DNA is initially bound by RPA and formation of RPA foci is therefore a standard marker of DNA resection. Cells were transfected with Cas9-HE, Cas9-CtIP or Cas9 as well as with siRNA directed towards CtIP or control. Two days after transfection, cells were X-ray irradiated to induce DSBs and RPA foci counted at 1, 2, 4, 6 and 8 h afterwards (FIG. 6). CtIP knock-down mildly decreased RPA foci formation (p<0.0005) while none of the Cas9 versions i.e. Cas9, Cas9-CtIP nor Cas9-HE significantly affected RPA foci formation. These results suggest that overexpression of Cas9-HE does not interfere with endogenous CtIP activity and does not seem to perturb the cell's general ability to cope with DNA double strand breaks.

### Example 7—Cas9-HE Induces a Different Pattern of Indels than Cas9

Recent studies have indicated that the pattern of indels induced by Cas9 is not random and is determined by the spacer sequence rather than genomic context (van Overbeek et al.; 2016). In addition, the mutation pattern could be modified by the DNA-PK inhibitor NU7441, which inhibits end-joining by cNHEJ, suggesting that the mutation pattern is dependent on the DNA repair pathways that have been involved.
Therefore it was assessed whether Cas9-HE induces a different pattern of indels than Cas9. Two guide RNAs, Spacer 54 and Spacer 93 targeting JAK and PCSK genes respectively, that were previously characterized by van Overbeek et al (2016) and the T2 guide RNA targeting the AAVS1 locus were tested in HEK293 cells and the mutation pattern determined by deep sequencing of PCR products of the target loci (see Tables 2 and 3 below).
Indel mutation patterns induced after transfection of nucleases and guide RNA expression vectors were determined by sequencing of PCR amplicons of the targeted region. When indicated, cells were treated with 10 μM DNA-PK inhibitor NU7441.
The indels shown are indels that represented more than 2% of mutant reads obtained with Cas9 or Cas9-HE in the absence of drug. If present, microhomologies (MH) of 2 or more nucleotides flanking the deletion are indicated.
Spacer 54 and Spacer 93 are from guide RNAs previously analyzed by van Overbeek et al. (2016). For spacer 54, mutant reads were 35.7% (of total 47199 reads), 29.8% (of total 48265 reads) and 6.5% (of total 116354 reads) for Cas9, Cas9-HE and Cas9+NU7441 respectively. For spacer 93, mutant reads were 31.3% (of total 45398 reads), 24.2% (of total 55573 reads) and 4.1% (of total 36979 reads) for Cas9, Cas9-HE and Cas9+NU7441 respectively. For T2 guide RNA, mutant reads were 39% (of total 68852 reads), 16.8% (of total 67815 reads) and 31.8% (of total 69696 reads) for Cas9, Cas9-HE and Cas9+NU7441 respectively.

TABLE 2
Indel mutation patterns induced by Cas9, Cas9-HE and Cas9+NU7441

| SEQ ID NO. | JAK (spacer 54) | Indel | MH | Cas9 | Cas9-HE | Cas9+NU7441 |
|---|---|---|---|---|---|---|
| 49 | TCCAGGTTCACCTCAGTCTTCTTGGAGCTCCTCATTTTAG | size | motif | (a) | (b) | (c) |
| 50 | TCCAGGTTCACCTCAGtTCTTCTTGGAGCTCCTCATTTTAG | 1 | | 20.7% | 12.9% | 1.6% |
| 51 | TCCAGGTTCACCTCAG--TTCTTGGAGCTCCTCATTTTAG | -2 | | 8.5% | 3.9% | 0.5% |
| 52 | TCCAGGTTCACC----TCTTCTTGGAGCTCCTCATTTTAG | -4 | TC | 7.3% | 11.0% | 10.0% |
| 53 | TCCAGGTTCA-------------------CCTCATTTTAG | -19 | CCTCA | 5.4% | 6.6% | 17.0% |
| 54 | TCCAGGTTCACCTCAG-CTTCTTGGAGCTCCTCATTTTAG | -1 | | 2.9% | 1.7% | 1.7% |
| 55 | TCCAGGTTCACCTCAG---TCTTGGAGCTCCTCATTTTAG | -3 | TCT | 2.7% | 1.4% | <0.1% |
| 56 | TCCAGGTTCAC------CTTCTTGGAGCTCCTCATTTTAG | -6 | CT | 2.5% | 4.0% | 3.7% |
| 57 | TCCAGG------------TTCTTGGAGCTCCTCATTTTAG | -12 | TTC | 1.9% | 2.2% | 3.8% |
| 58 | TCCAGGTTCACC-------TCTTGGAGCTCCTCATTTTAG | -7 | TC | 1.8% | 4.0% | 6.5% |
| SEQ ID NO. | PCSK (spacer 93) | Indel | MH | Cas9 | Cas9-HE | Cas9+NU7441 |
| 59 | GAGCTTTAAAATGGTTCCGACTTGTCCCTCTCTCAGCCCTC | size | motif | (a) | (b) | (c) |
| 60 | GAGCTTTAAAATGGTTCCGACtTTGTCCCTCTCTCAGCCCTC | 1 | | 26.8% | 16.4% | 8.3% |
| 61 | GAGCTTTAAAAT-----------GTCCCTCTCTCAGCCCTC | -10 | GT | 11.1% | 16.0% | 7.5% |
| 62 | GAGCTTTAAAATGGTTCCGAC-TGTCCCTCTCTCAGCCCTC | -1 | | 8.8% | 6.4% | 0.4% |
| 63 | GAGCTTTAAAATGGTTCCGA---------CTCTCAGCCCTC | -9 | CT | 4.0% | 5.9% | 11.9% |
| 64 | GAGCTTTAAAATGGT---------TCCCTCTCTCAGCCCTC | -9 | TCC | 2.4% | 2.6% | 1.8% |
| 65 | GAGCTTTAAAATGGT------------------------TC | -24 | TCC | 2.2% | 2.9% | 14.7% |
| 66 | GAGCTTTAAAA-----------TGTCCCTCTCTCAGCCCTC | -11 | TG | 1.9% | 2.6% | 0.8% |
| SEQ ID NO. | AAVS1 | Indel | MH | Cas9 | Cas9-HE | Cas9+NU7441 |
| 67 | AAGGATGGGGCTTTTCTGTCACCAATCCTGTCCCTAGTGGC | size | motif | (a) | (b) | (c) |
| 68 | AAGGATGGGGCTTTTCTGTCACCAAT-CTGTCCCTAGTGGC | -1 | | 40.9% | 26.9% | 27.5% |
| 69 | AAGGATGGGGCTTTTCTGTCACCAATCC-GTCCCTAGTGGC | -1 | | 9.7% | 4.1% | 5.4% |
| 70 | AAGGATGGGGCTTTT------------CTGTCCCTAGTGGC | -12 | CTGTC | 4.8% | 10.0% | 8.8% |
| 71 | AAGGATGGGGCTTTTCTGTCACCAATcCCTGTCCCTAGTGGC | 1 | | 5.5% | 3.3% | 3.6% |
| 72 | AAGGATGGGGCTTTTCTGTCACCAA-----TCCCTAGTGGC | -5 | TCC | 3.9% | 8.0% | 9.4% |
| 73 | AAGGATGGGGCTTTTCTGTCACCAATC--GTCCCTAGTGGC | -2 | | 2.7% | 5.0% | 2.9% |
| 74 | AAGGATGGGGCTTTTCTGTCACCAATCctgCTGTCCCTAGTGGC | 3 | | 2.1% | 4.0% | 1.9% |
| 75 | AAGGATGGGGCTTTTCTGTCA-----------CCTAGTGGC | -11 | CC | 0.9% | 2.4% | 2.9% |

TABLE 3
Indel mutation patterns induced by Cas9, Cas9-HE and Cas9+NU7441

| SEQ ID NO. | JAK (spacer 54) | Indel | MH | (c′)/ | (b)/ |
|---|---|---|---|---|---|
| 49 | TCCAGGTTCACCTCAGTCTTCTTGGAGCTCCTCATTTTAG | size | motif | (a) | (a) |
| 50 | TCCAGGTTCACCTCAGtTCTTCTTGGAGCTCCTCATTTTAG | 1 | | 0.1 | 0.6 |
| 51 | TCCAGGTTCACCTCAG--TTCTTGGAGCTCCTCATTTTAG | -2 | | 0.1 | 0.5 |
| 52 | TCCAGGTTCACC----TCTTCTTGGAGCTCCTCATTTTAG | -4 | TC | 1.4 | 1.5 |
| 53 | TCCAGGTTCA-------------------CCTCATTTTAG | -19 | CCTCA | 3.1 | 1.2 |
| 54 | TCCAGGTTCACCTCAG-CTTCTTGGAGCTCCTCATTTTAG | -1 | | 0.6 | 0.6 |
| 55 | TCCAGGTTCACCTCAG---TCTTGGAGCTCCTCATTTTAG | -3 | TCT | <0.03 | 0.5 |
| 56 | TCCAGGTTCAC------CTTCTTGGAGCTCCTCATTTTAG | -6 | CT | 1.5 | 1.6 |
| 57 | TCCAGG------------TTCTTGGAGCTCCTCATTTTAG | -12 | TTC | 2.0 | 1.1 |
| 58 | TCCAGGTTCACC-------TCTTGGAGCTCCTCATTTTAG | -7 | TC | 3.6 | 2.2 |
| SEQ ID NO. | PCSK (spacer 93) | Indel | MH | (c′)/ | (b)/ |
| 59 | GAGCTTTAAAATGGTTCCGACTTGTCCCTCTCTCAGCCCTC | size | motif | (a) | (a) |
| 60 | GAGCTTTAAAATGGTTCCGACtTTGTCCCTCTCTCAGCCCTC | 1 | | 0.3 | 0.6 |
| 61 | GAGCTTTAAAAT-----------GTCCCTCTCTCAGCCCTC | -10 | GT | 0.7 | 1.4 |
| 62 | GAGCTTTAAAATGGTTCCGAC-TGTCCCTCTCTCAGCCCTC | -1 | | 0.04 | 0.7 |
| 63 | GAGCTTTAAAATGGTTCCGA---------CTCTCAGCCCTC | -9 | CT | 3.0 | 1.5 |
| 64 | GAGCTTTAAAATGGT---------TCCCTCTCTCAGCCCTC | -9 | TCC | 0.8 | 1.1 |
| 65 | GAGCTTTAAAATGGT------------------------TC | -24 | TCC | 6.8 | 1.4 |
| 66 | GAGCTTTAAAA-----------TGTCCCTCTCTCAGCCCTC | -11 | TG | 0.4 | 1.3 |
| SEQ ID NO. | AAVS1 | Indel | MH | (c′)/ | (b)/ |
| 67 | AAGGATGGGGCTTTTCTGTCACCAATCCTGTCCCTAGTGGC | size | motif | (a) | (a) |
| 68 | AAGGATGGGGCTTTTCTGTCACCAAT-CTGTCCCTAGTGGC | -1 | | 0.7 | 0.7 |
| 69 | AAGGATGGGGCTTTTCTGTCACCAATCC-GTCCCTAGTGGC | -1 | | 0.4 | 0.6 |
| 70 | AAGGATGGGGCTTTT------------CTGTCCCTAGTGGC | -12 | CTGTC | 2.1 | 1.8 |
| 71 | AAGGATGGGGCTTTTCTGTCACCAATcCCTGTCCCTAGTGGC | 1 | | 0.6 | 0.7 |
| 72 | AAGGATGGGGCTTTTCTGTCACCAA-----TCCCTAGTGGC | -5 | TCC | 2.0 | 2.4 |
| 73 | AAGGATGGGGCTTTTCTGTCACCAATC--GTCCCTAGTGGC | -2 | | 1.9 | 1.1 |
| 74 | AAGGATGGGGCTTTTCTGTCACCAATCctgCTGTCCCTAGTGGC | 3 | | 1.9 | 0.9 |
| 75 | AAGGATGGGGCTTTTCTGTCA-----------CCTAGTGGC | -11 | CC | 3.1 | 2.6 |

The proportion of mutant reads obtained with Cas9-HE and Cas9 were similar for Spacer 54 and Spacer 93, while for guide T2, Cas9-HE gave approximately 50% fewer mutant reads than Cas9. The indels representing more than 2% of mutant reads for Cas9 and Cas9-HE were examined in detail. Depending on the guide RNA, they corresponded to 7 to 9 different indels that taken all together represented 47 to 70% of total mutant reads. Interestingly, for all three guides, it was observed that the patterns of indels induced by Cas9-HE were different from those induced by Cas9. The extent of changes, however, depended on the guide RNA (Tables 2 and 3). As a control, the NU7441 treatment of Cas9 transfected cells that was previously reported by van Overbeek et al. (2016) was repeated. Interestingly, for all three guide RNAs, Cas9-HE and NU7441 treatment resulted for most indels in similar types of changes compared to Cas9 (changes were similar for 20 out of 24 indels). The differences, however, were generally of greater amplitude with NU7441. In particular, for spacer 54, the two most frequent mutations observed with Cas9 were reduced 10-fold by NU7441 treatment but only 2-fold when using Cas9-HE. This is reminiscent of the effects of lower NU7441 doses observed by van Overbeek et al (2016). It was also noted that indels with increased frequency were almost all deletions flanked by microhomologies. When comparing Cas9-HE to Cas9, 13 out 14 indels with increased frequency were deletions flanked by micro-homologies of 2 or more nucleotides and 10 out of 12 for NU7441 treatment. Taken together, these results are consistent with Cas9-HE inducing a different balance of end-joining pathways compared to Cas9 and having an effect similar to a low NU7441 dose, with a partial inhibition of cNHEJ and an increase of MMEJ, likely due to stimulation of resection by the HE domain.
During homologous recombination, CtIP and the MRN complex trigger end resection at the DSB, generating single stranded DNA needed to search for and copy a DNA repair template. CtIP is also known to contribute to alternative endjoining, which requires resection and is mechanistically different from cNHEJ. Similarly, Cas9-HE may stimulate DSB repair by HR, as suggested by elevated transgene integration, as well as favor alternative end joining pathways. Indeed, the mutation patterns were different for Cas9-HE and Cas9, suggesting that the balance of cNHEJ and MMEJ end joining pathways is affected by the fusion of the HE domain to Cas9. The effect of Cas9-HE was reminiscent of the effects of low NU7441 dose reported by van Overbeek et al (2016), suggesting that the HE domain may exert a mild inhibition of cNHEJ. In addition, deletions flanked by microhomologies had increased frequency with Cas9-HE (Tables 2 and 3), suggesting that MMEJ was favored relative to cNHEJ. These findings are consistent with the known role of CtIP in triggering DNA resection and antagonizing cNHEJ at the earlier steps of choice between the DSB repair pathways. The increased role of MMEJ may explain why, even though transgene integration is stimulated, the frequency of indels is not significantly different with Cas9-HE compared to Cas9.

### Example 8—HDR Stimulation Depends on the Guide RNA

When experiments were performed in rats, transgene integration was increased at the Rosa26 locus. 5 additional target loci in human HEK293 cells were tested and it was found that Cas9-HE stimulated more efficient transgene integration at 4 of the 5 sites tested (FIG. 7A). Several non-exclusive explanations could be considered to explain why integration was not stimulated at some targets, including a specific role of the target sequence or chromatin context. The possibility was examined that the guide RNA could play a role in determining whether Cas9-HE will stimulate HDR more efficiently than Cas9.
3 guide RNAs were compared, which all target cleavage in a short 50 bp sequence of the AAVS1 locus. The homology arms in the donor DNA used in the experiments above were first slightly shortened to avoid potential cleavage by the guide RNAs and so that the same donor DNA could be used with all 3 guides.
The sequences used in this assay are the following:

Spacer sequence of guide T2
(SEQ ID NO. 78)
GGGGCCACUAGGGACAGGAU
Target sequence of guide T2
(SEQ ID NO. 79)
GGGGCCACTAGGGACAGGATTGG
DNA sequence of guide T2:
(SEQ ID NO. 46)
GGGGCCACTAGGGACAGGATgttttagagctagaaatagcaagttaaaat
aaggctagtccgttatcaacttgaaaaagtggcaccgagtcggtgc
Spacer sequence of guide T4
(SEQ ID NO. 80)
GACAGAAAAGCCCCAUCCUUUU
Target sequence of guide T4
(SEQ ID NO. 81)
GACAGAAAAGCCCCATCCTTTTGGG
DNA sequence of guide T4:
(SEQ ID NO. 82)
GACAGAAAAGCCCCATCCTTTTgttttagagctagaaatagcaagttaaa
ataaggctagtccgttatcaacttgaaaaagtggcaccgagtcggtgc
Spacer sequence of guide D1
(SEQ ID NO. 83)
GACUAGGAAGGGUUAGACCCAAAAGGA
Target sequence of guide D1
(SEQ ID NO. 84)
GACTAGGAAGGGTTAGACCCAAAAGGATGG
DNA sequence of guide D1:
(SEQ ID NO. 85)
gactaggaagggttagacccaaaaggagttttagagctagaaatagcaag
ttaaaataaggctagtccgttatcaacttgaaaaagtggcaccgagtcgg
tgc

It is to be noted that in the DNA sequences of the guide RNAs, the lowercases represent the constant part of the guide RNA and the uppercases represent the spacer sequences that determine the DNA target sequence of the complex between guide RNA and Cas9.
When Cas9-HE and Cas9 were compared with the different guides and modified donor, it was found that Cas9-HE directed approximately 2-fold higher levels of transgene integration than Cas9 for guides T2, T4 and D1 (FIG. 7B). The results indicate that, unexpectedly, the stimulation of HDR by Cas9-HE is dependent on the guide RNA used to trigger genome editing.

### REFERENCES

### Non-Patent References

Aliyari R, Ding S W. RNA-based viral immunity initiated by the Dicer family of host immune receptors. Immunol Rev. 2009 January;227(1):176-88.

Altschul S F, Madden T L, Schaffer A A, Zhang J, Zhang Z, Miller W, Lipman D J.

Gapped BLAST and PSI-BLAST: a new generation of protein database search programs. Nucleic Acids Res. 1997 Sep. 1;25(17):3389-402.

Bedell V M, Wang Y, Campbell J M, Poshusta T L, Starker C G, Krug R G 2nd, Tan W, Penheiter S G, Ma A C, Leung A Y, Fahrenkrug S C, Carlson D F, Voytas D F, Clark K J, Essner J J, Ekker S C. In vivo genome editing using a high-efficiency TALEN system. Nature. 2012 Nov. 1;491(7422):114-8.

Cass S D, Haas K A, Stoll B, Alkhnbashi O S, Sharma K, Urlaub H, Backofen R, Marchfelder A, Bolt E L. The role of Cas8 in type I CRISPR interference. Biosci Rep. 2015 May 5;35(3). pii: e00197.

Chaikind B, Bessen J L, Thompson D B, Hu J H, Liu D R. A programmable Cas9-serine recombinase fusion protein that operates on DNA sequences in mammalian cells. Nucleic Acids Res. 2016 Nov. 16;44(20):9758-9770.

Chenouard, V., Brusselle, L., Heslan, J. M., Remy, S., Menoret, S., Usal, C., Ouisse, L. H., TH, N. G., Anegon, I., and Tesson, L. (2016). A Rapid and Cost-Effective Method for Genotyping Genome-Edited Animals: A Heteroduplex Mobility Assay Using Microfluidic Capillary Electrophoresis. J Genet Genomics 43, 341-348.

Chylinski K, Le Rhun A, Charpentier E. The tracrRNA and Cas9 families of type II CRISPR-Cas immunity systems. RNA Biol. 2013 May;10(5):726-37.

Chylinski K, Makarova K S, Charpentier E, Koonin E V. Classification and evolution of type II CRISPR-Cas systems. Nucleic Acids Res. 2014 June;42(10):6091-105.

Davies, O. R., Forment, J. V., Sun, M., Belotserkovskaya, R., Coates, J., Galanty, Y., Demir, M., Morton, C. R., Rzechorzek, N. J., Jackson, S. P., Pellegrini, L., 2015. CtIP tetramer assembly is required for DNA-end resection and repair. Nat. Struct. Mol. Biol. 22, 150-157.

DeKelver R C, Choi V M, Moehle E A, Paschon D E, Hockemeyer D, Meijsing S H, Sancak Y, Cui X, Steine E J, Miller J C, Tam P, Bartsevich V V, Meng X, Rupniewski I, Gopalan S M, Sun H C, Pitz K J, Rock J M, Zhang L, Davis G D, Rebar E J, Cheeseman I M, Yamamoto K R, Sabatini D M, Jaenisch R, Gregory P D, Urnov F D. Functional genomics, proteomics, and regulatory DNA analysis in isogenic settings using zinc finger nuclease-driven transgenesis into a safe harbor locus in the human genome. Genome Res. 20, 1133-1142 (2010).

Deltcheva, E., Chylinski, K., Sharma, C. M., Gonzales, K., Chao, Y., Pirzada, Z. A., Eckert, M. R., Vogel, J., Charpentier, E., 2011. CRISPR RNA maturation by trans-encoded small RNA and host factor RNase III. Nature 471, 602-607.

Ding Q, Lee Y K, Schaefer E A, Peters D T, Veres A, Kim K, Kuperwasser N, Motola D L, Meissner T B, Hendriks W T, Trevisan M, Gupta R M, Moisan A, Banks E, Friesen M, Schinzel R T, Xia F, Tang A, Xia Y, Figueroa E, Wann A, Ahfeldt T, Daheron L, Zhang F, Rubin L L, Peng L F, Chung R T, Musunuru K, Cowan C A. A TALEN genome-editing system for generating human stem cell-based disease models. Cell Stem Cell. 2013 Feb. 7;12(2):238-51.

Doyon, Y., McCammon, J. M., Miller, J. C., Faraji, F., Ngo, C., Katibah, G. E., Amora, R., Hocking, T. D., Zhang, L., Rebar, E. J., Gregory, P. D., Urnov, F. D., Amacher, S. L., 2008. Heritable targeted gene disruption in zebrafish using designed zinc-finger nucleases. Nat. Biotechnol. 26, 702-708.

Dujon, B., 1989. Group I introns as mobile genetic elements: facts and mechanistic speculations—a review. Gene 82, 91-114.

Esvelt K M, Mali P, Braff J L, Moosburner M, Yaung S J, Church G M. Orthogonal Cas9 proteins for RNA-guided gene regulation and editing. Nat Methods. 2013 November;10(11):1116-21.

Gandia, M., Xu, S., Font, C., Marcos, J. F., 2016. Disruption of ku70 involved in non-homologous end joining facilitates homologous recombination but increases temperature sensitivity in the phytopathogenic fungus Penicillium digitatum. Fungal Biol. 120, 317-323.

Gasiunas G, Barrangou R, Horvath P, Siksnys V. Cas9-crRNA ribonucleoprotein complex mediates specific DNA cleavage for adaptive immunity in bacteria. Proc Natl Acad Sci USA. 2012 Sep. 25;109(39):E2579-86.

Guilinger J P, Thompson D B, Liu D R. Fusion of catalytically inactive Cas9 to FokI nuclease improves the specificity of genome modification. Nat Biotechnol. 2014 June;32(6):577-582.

Gutschner, T., Haemmerle, M., Genovese, G., Draetta, G. F., Chin, L., 2016. Post-translational Regulation of Cas9 during G1 Enhances Homology-Directed Repair. Cell Rep. 14, 1555-1566.

Heler R, Samai P, Modell J W, Weiner C, Goldberg G W, Bikard D, Marraffini L A. Cas9 specifies functional viral targets during CRISPR-Cas adaptation. Nature. 2015 Mar. 12;519(7542):199-202.

Howden, S. E., McColl, B., Glaser, A., Vadolas, J., Petrou, S., Little, M. H., Elefanty, A. G., Stanley, E. G., 2016. A Cas9 Variant for Efficient Generation of Indel-Free Knockin or Gene-Corrected Human Pluripotent Stem Cells. Stem Cell Rep. 2016 Sep 13;7(3):508-517.

Hsu P D, Scott D A, Weinstein J A, Ran F A, Konermann S, Agarwala V, Li Y, Fine E J, Wu X, Shalem O, Cradick T J, Marraffini L A, Bao G, Zhang F. DNA targeting specificity of RNA-guided Cas9 nucleases. Nat Biotechnol. 2013 September;31(9):827-32.

Huang, P., Xiao, A., Zhou, M., Zhu, Z., Lin, S., Zhang, B., 2011. Heritable gene targeting in zebrafish using customized TALENs. Nat. Biotechnol. 29, 699-700.

Joung J K, Sander J D. TALENs: a widely applicable technology for targeted genome editing. Nat Rev Mol Cell Biol. 2013 January;14(1):49-55.

Kleinstiver B P, Pattanayak V, Prew M S, Tsai S Q, Nguyen N T, Zheng Z, Joung J K. High-fidelity CRISPR-Cas9 nucleases with no detectable genome-wide off-target effects. Nature. 2016 Jan. 28;529(7587):490-5.

Köferle A, Worf K, Breunig C, Baumann V, Herrero J, Wiesbeck M, Hutter L H, Götz M, Fuchs C, Beck S, Stricker S H. CORALINA: a universal method for the generation of gRNA libraries for CRISPR-based screening. BMC Genomics. 2016 Nov. 14;17(1):917.

Komatsu, K., 2016. NBS1 and multiple regulations of DNA damage response. J. Radiat. Res. (Tokyo) 57 Suppl 1, i11-i17.

Kosugi S, Hasebe M, Matsumura N, Takashima H, Miyamoto-Sato E, Tomita M, Yanagawa H. Six classes of nuclear localization signals specific to different binding grooves of importin alpha. J Biol Chem. 2009 Jan. 2;284(1):478-85.

Lange A, Mills R E, Lange C J, Stewart M, Devine S E, Corbett A H. Classical nuclear localization signals: definition, function, and interaction with importin alpha. J Biol Chem. 2007 Feb. 23;282(8):5101-5.

Liu, T., Huang, J., 2016. DNA End Resection: Facts and Mechanisms. Genomics Proteomics Bioinformatics 14, 126-130.

Makarova K S, Aravind L, Wolf Y I, Koonin E V. Unification of Cas protein families and a simple scenario for the origin and evolution of CRISPR-Cas systems. Biol Direct. 2011 Jul. 14;6:38.

Mali P, Yang L, Esvelt K M, Aach J, Guell M, DiCarlo J E, Norville J E, Church G M. RNA-guided human genome engineering via Cas9. Science. 2013 Feb. 15;339(6121):823-6.

Marfori M, Mynott A, Ellis J J, Mehdi A M, Saunders N F, Curmi P M, Forwood J K, Boden M, Kobe B. Molecular basis for specificity of nuclear import and prediction of nuclear localization. Biochim Biophys Acta. 2011 September;1813(9):1562-77.

Menoret, S., De Cian, A., Tesson, L., Remy, S., Usal, C., Boule, J. B., Boix, C., Fontaniere, S., Creneguy, A., Nguyen, T. H., Brusselle, L., Thinard, R., Gauguier, D., Concordet, J. P., Cherifi, Y., Fraichard, A., Giovannangeli, C., Anegon, I. (2015). Homology-directed repair in rodent zygotes using Cas9 and TALEN engineered proteins. Sci Rep 5, 14410.

Miller J C, Holmes M C, Wang J, Guschin D Y, Lee Y L, Rupniewski I, Beausejour C M, Waite A J, Wang N S, Kim K A, Gregory P D, Pabo C O, Rebar E J. An improved zinc-finger nuclease architecture for highly specific genome editing. Nat Biotechnol. 2007 July;25(7):778-85.

Miller J C, Tan S, Qiao G, Barlow K A, Wang J, Xia D F, Meng X, Paschon D E, Leung E, Hinkley S J, Dulay G P, Hua K L, Ankoudinova I, Cost G J, Urnov F D, Zhang H S, Holmes M C, Zhang L, Gregory P D, Rebar E J. A TALE nuclease architecture for efficient genome editing. Nat Biotechnol. 2011 February;29(2):143-8.

Moll J R, Ruvinov S B, Pastan I, Vinson C. Designed heterodimerizing leucine zippers with a ranger of pIs and stabilities up to 10(-15) M. Protein Sci. 2001 March;10(3):649-55.

Perez E E, Wang J, Miller J C, Jouvenot Y, Kim K A, Liu O, Wang N, Lee G, Bartsevich V V, Lee Y L, Guschin D Y, Rupniewski I, Waite A J, Carpenito C, Carroll R G, Orange J S, Urnov F D, Rebar E J, Ando D, Gregory P D, Riley J L, Holmes M C, June C H. Establishment of HIV-1 resistance in CD4+ T cells by genome editing using zinc-finger nucleases. Nat Biotechnol. 2008 July;26(7):808-16.

Piganeau, M., Ghezraoui, H., De Cian, A., Guittat, L., Tomishima, M., Perrouault, L., René, O., Katibah, G. E., Zhang, L., Holmes, M. C., Doyon, Y., Concordet, J. -P., Giovannangeli, C., Jasin, M., Brunet, E., 2013. Cancer translocations in human cells induced by zinc finger and TALE nucleases. Genome Res. 23, 1182-1193.

Plessis, A., Perrin, A., Haber, J. E., Dujon, B., 1992. Site-specific recombination determined by I-SceI, a mitochondrial group I intron-encoded endonuclease expressed in the yeast nucleus. Genetics 130, 451-460.

Reddington S C, Howarth M. Secrets of a covalent interaction for biomaterials and biotechnology: SpyTag and SpyCatcher. Curr Opin Chem Biol. 2015 December;29:94-9.

Remy S, Tesson L, Menoret S, Usal C, De Cian A, Thepenier V, Thinard R, Baron D, Charpentier M, Renaud J B, Buelow R, Cost G J, Giovannangeli C, Fraichard A, Concordet J P, Anegon I. Efficient gene targeting by homology-directed repair in rat zygotes using TALE nucleases. Genome Res. 2014 August;24(8):1371-83.

Renaud J B, Boix C, Charpentier M, De Cian A, Cochennec J, Duvernois-Berthet E, Perrouault L, Tesson L, Edouard J, Thinard R, Cherifi Y, Menoret S, Fontanière S, de Crozé N, Fraichard A, Sohm F, Anegon I, Concordet J P, Giovannangeli C. Improved Genome Editing Efficiency and Flexibility Using Modified Oligonucleotides with TALEN and CRISPR-Cas9 Nucleases. Cell Rep. 14, 2263-2272 (2016).

Reyon D, Tsai S Q, Khayter C, Foden J A, Sander J D, Joung J K. FLASH assembly of TALENs for high-throughput genome editing. Nat Biotechnol. 2012 May;30(5):460-5.

Rouet, P., Smih, F., Jasin, M., 1994. Expression of a site-specific endonuclease stimulates homologous recombination in mammalian cells. Proc. Natl. Acad. Sci. U.S.A. 91, 6064-6068.

Savic D, Partridge E C, Newberry K M, Smith S B, Meadows S , Roberts B S, Mackiewicz M, Mendenhall E M, Myers R M. CETCh-seq: CRISPR epitope tagging ChIP-seq of DNA-binding proteins. Genome Res. 2015 October;25(10):1581-9.

Shah N H, Muir T W. Inteins: Nature's Gift to Protein Chemists. Chem Sci. 2014;5(1):446-461.

Sinkunas T, Gasiunas G, Fremaux C, Barrangou R, Horvath P, Siksnys V. Cas3 is a single-stranded DNA nuclease and ATP-dependent helicase in the CRISPR/Cas immune system. EMBO J. 2011 Apr.6;30(7):1335-42.

Slaymaker I M, Gao L, Zetsche B, Scott D A, Yan W X, Zhang F. Rationally engineered Cas9 nucleases with improved specificity. Science. 2016 Jan. 1;351(6268):84-8.

van Overbeek M, Capurso D, Carter M M, Thompson M S, Frias E, Russ C, Reece-Hoyes J S, Nye C, Gradia S, Vidal B, Zheng J, Hoffman G R, Fuller C K, May A P. DNA Repair Profiling Reveals Nonrandom Outcomes at Cas9-Mediated Breaks. Mol. Cell 63, 633-646 (2016).

Urnov F D, Rebar E J, Holmes M C, Zhang H S, Gregory P D. Genome editing with engineered zinc finger nucleases. Nat Rev Genet. 2010 September;11(9):636-46.

Wang, H., Shi, L. Z., Wong, C. C. L., Han, X., Hwang, P. Y. -H., Truong, L. N., Zhu, Q., Shao, Z., Chen, D. J., Berns, M. W., Yates, J. R., Chen, L., Wu, X. The interaction of CtIP and Nbs1 connects CDK and ATM to regulate HR-mediated double-strand break repair. PLoS Genet. 2013; 9, e1003277.

Wood A J, Lo T W, Zeitler B, Pickle C S, Ralston E J, Lee A H, Amora R, Miller J C, Leung E, Meng X, Zhang L, Rebar E J, Gregory P D, Urnov F D, Meyer B J. Targeted genome editing across species using ZFNs and TALENs. Science. 2011 Jul. 15;333(6040):307.

Yang, D., Scavuzzo, M. A., Chmielowiec, J., Sharp, R., Bajic, A., Borowiak, M., 2016. Enrichment of G2/M cell cycle phase in human pluripotent stem cells enhances HDR-mediated gene repair with customizable endonucleases. Sci. Rep. 6, 21264.

Zetsche B, Gootenberg J S, Abudayyeh O O, Slaymaker I M, Makarova K S, Essletzbichler P, Volz S E, Joung J, van der Oost J, Regev A, Koonin E V, Zhang F. Cpf1 is a single RNA-guided endonuclease of a class 2 CRISPR-Cas system. Cell. 2015 Oct. 22;163(3):759-71.

### Patent References

EP 2 368 982

WO 2012/138939

WO 2015/153889

WO 2015/153940

WO 2016/054326

WO 2016/183402

WO 2016/196805

LISTING OF THE SEQUENCES USED HEREIN

SEQ ID NO. 1 - sequence of Streptococcus pyogenes Cas9
MDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVL
DATLIHQSITGLYETRIDLSQLGGDSRAD
SEQ ID NO. 2 - sequence of human (Homo sapiens) CtIP
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQAAELECEEDVIPDSPIT
AFSFSGVNRLRRKENPHVRYIEQTHTKLEHSVCANEMRKVSKSSTHPQHNPNENEI
LVADTYDQSQSPMAKAHGTSSYTPDKSSFNLATVVAETLGLGVQEESETQGPMSP
LGDELYHCLEGNHKKQPFEESTRNTEDSLRFSDSTSKTPPQEELPTRVSSPVFGATS
SIKSGLDLNTSLSPSLLQPGKKKHLKTLPFSNTCISRLEKTRSKSEDSALFTHHSLGS
EVNKIIIQSSNKQILINKNISESLGEQNRTEYGKDSNTDKHLEPLKSLGGRTSKRKKT
EEESEHEVSCPQASFDKENAFPFPMDNQFSMNGDCVMDKPLDLSDRFSAIQRQEK
SQGSETSKNKFRQVTLYEALKTIPKGFSSSRKASDGNCTLPKDSPGEPCSQECIILQP
LNKCSPDNKPSLQIKEENAVFKIPLRPRESLETENVLDDIKSAGSHEPIKIQTRSDHG
GCELASVLQLNPCRTGKIKSLQNNQDVSFENIQWSIDPGADLSQYKMDVTVIDTK
DGSQSKLGGETVDMDCTLVSETVLLKMKKQEQKGEKSSNEERKMNDSLEDMFD
RTTHEEYESCLADSFSQAADEEEELSTATKKLHTHGDKQDKVKQKAFVEPYFKGD
ERETSLQNFPHIEVVRKKEERRKLLGHTCKECEIYYADMPAEEREKKLASCSRHRF
RYIPPNTPENFWEVGFPSTQTCMERGYIKEDLDPCPRPKRRQPYNAIFSPKGKEQKT
SEQ ID NO. 3 - tetramerization domain of human CtIP (22-45)
DLWTKLKECHDREVQGLQVKVTKL
SEQ ID NO. 4 - dimerization domain of human CtIP (46-166)
KQERILDAQRLEEFFTKNQQLREQQKVLHETIKVLEDRLRAGLCDRCAVTEEHMR
KKQQEFENIRQQNLKLITELMNERNTLQEENKKLSEQLQQKIENDQQHQAAELEC
EEDVIPDSPIT
SEQ ID NO. 5 - 1-149 domain of human CtIP
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQ
SEQ ID NO. 6 - 1-296 (HE) domain of human CtIP
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQAAELECEEDVIPDSPIT
AFSFSGVNRLRRKENPHVRYIEQTHTKLEHSVCANEMRKVSKSSTHPQHNPNENEI
LVADTYDQSQSPMAKAHGTSSYTPDKSSFNLATVVAETLGLGVQEESETQGPMSP
LGDELYHCLEGNHKKQPFE
SEQ ID NO. 7- 1-416 domain of human CtIP
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQAAELECEEDVIPDSPIT
AFSFSGVNRLRRKENPHVRYIEQTHTKLEHSVCANEMRKVSKSSTHPQHNPNENEI
LVADTYDQSQSPMAKAHGTSSYTPDKSSFNLATVVAETLGLGVQEESETQGPMSP
LGDELYHCLEGNHKKQPFEESTRNTEDSLRFSDSTSKTPPQEELPTRVSSPVFGATS
SIKSGLDLNTSLSPSLLQPGKKKHLKTLPFSNTCISRLEKTRSKSEDSALFTHHSLGS
EVNKIIIQSSNKQILINKNISESL
SEQ ID NO. 8 - 1-669 domain of CtIP
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQAAELECEEDVIPDSPIT
AFSFSGVNRLRRKENPHVRYIEQTHTKLEHSVCANEMRKVSKSSTHPQHNPNENEI
LVADTYDQSQSPMAKAHGTSSYTPDKSSFNLATVVAETLGLGVQEESETQGPMSP
LGDELYHCLEGNHKKQPFEESTRNTEDSLRFSDSTSKTPPQEELPTRVSSPVFGATS
SIKSGLDLNTSLSPSLLQPGKKKHLKTLPFSNTCISRLEKTRSKSEDSALFTHHSLGS
EVNKIIIQSSNKQILINKNISESLGEQNRTEYGKDSNTDKHLEPLKSLGGRTSKRKKT
EEESEHEVSCPQASFDKENAFPFPMDNQFSMNGDCVMDKPLDLSDRFSAIQRQEK
SQGSETSKNKFRQVTLYEALKTIPKGFSSSRKASDGNCTLPKDSPGEPCSQECIILQP
LNKCSPDNKPSLQIKEENAVFKIPLRPRESLETENVLDDIKSAGSHEPIKIQTRSDHG
GCELASVLQLNPCRTGKIKSLQNNQDVSFENIQWSIDPGADLSQYKMD
SEQ ID NO. 9 - 1-790 domain of human CtIP (deltaSD)
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQAAELECEEDVIPDSPIT
AFSFSGVNRLRRKENPHVRYIEQTHTKLEHSVCANEMRKVSKSSTHPQHNPNENEI
LVADTYDQSQSPMAKAHGTSSYTPDKSSFNLATVVAETLGLGVQEESETQGPMSP
LGDELYHCLEGNHKKQPFEESTRNTEDSLRFSDSTSKTPPQEELPTRVSSPVFGATS
SIKSGLDLNTSLSPSLLQPGKKKHLKTLPFSNTCISRLEKTRSKSEDSALFTHHSLGS
EVNKIIIQSSNKQILINKNISESLGEQNRTEYGKDSNTDKHLEPLKSLGGRTSKRKKT
EEESEHEVSCPQASFDKENAFPFPMDNQFSMNGDCVMDKPLDLSDRFSAIQRQEK
SQGSETSKNKFRQVTLYEALKTIPKGFSSSRKASDGNCTLPKDSPGEPCSQECIILQP
LNKCSPDNKPSLQIKEENAVFKIPLRPRESLETENVLDDIKSAGSHEPIKIQTRSDHG
GCELASVLQLNPCRTGKIKSLQNNQDVSFENIQWSIDPGADLSQYKMDVTVIDTK
DGSQSKLGGETVDMDCTLVSETVLLKMKKQEQKGEKSSNEERKMNDSLEDMFD
RTTHEEYESCLADSFSQAADEEEELSTATKKLHTHGDKQDKVKQKAFVEPYFKGD
ERETSL
SEQ ID NO. 10 - 416-897 domain of human CtIP
LGEQNRTEYGKDSNTDKHLEPLKSLGGRTSKRKKTEEESEHEVSCPQASFDKENA
FPFPMDNQFSMNGDCVMDKPLDLSDRFSAIQRQEKSQGSETSKNKFRQVTLYEAL
KTIPKGFSSSRKASDGNCTLPKDSPGEPCSQECIILQPLNKCSPDNKPSLQIKEENAV
FKIPLRPRESLETENVLDDIKSAGSHEPIKIQTRSDHGGCELASVLQLNPCRTGKIKS
LQNNQDVSFENIQWSIDPGADLSQYKMDVTVIDTKDGSQSKLGGETVDMDCTLVS
ETVLLKMKKQEQKGEKSSNEERKMNDSLEDMFDRTTHEEYESCLADSFSQAADE
EEELSTATKKLHTHGDKQDKVKQKAFVEPYFKGDERETSLQNFPHIEVVRKKEER
RKLLGHTCKECEIYYADMPAEEREKKLASCSRHRFRYIPPNTPENFWEVGFPSTQT
CMERGYIKEDLDPCPRPKRRQPYNAIFSPKGKEQKT
SEQ ID NO. 11 - 669-897 domain of human CtIP
DVTVIDTKDGSQSKLGGETVDMDCTLVSETVLLKMKKQEQKGEKSSNEERKMND
SLEDMFDRTTHEEYESCLADSFSQAADEEEELSTATKKLHTHGDKQDKVKQKAFV
EPYFKGDERETSLQNFPHIEVVRKKEERRKLLGHTCKECEIYYADMPAEEREKKLA
SCSRHRFRYIPPNTPENFWEVGFPSTQTCMERGYIKEDLDPCPRPKRRQPYNAIFSP
KGKEQKT
SEQ ID NO. 12 - 1-170 domain of human CtIP (HE1)
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQAAELECEEDVIPDSPIT
AFSF
SEQ ID NO. 13 - 46-296 domain of human CtIP (HE2)
KQERILDAQRLEEFFTKNQQLREQQKVLHETIKVLEDRLRAGLCDRCAVTEEHMR
KKQQEFENIRQQNLKLITELMNERNTLQEENKKLSEQLQQKIENDQQHQAAELEC
EEDVIPDSPITAFSFSGVNRLRRKENPHVRYIEQTHTKLEHSVCANEMRKVSKSSTH
PQHNPNENEILVADTYDQSQSPMAKAHGTSSYTPDKSSFNLATVVAETLGLGVQE
ESETQGPMSPLGDELYHCLEGNHKKQPFE
SEQ ID NO. 14 - 166-296 domain of human CtIP (HE3)
TAFSFSGVNRLRRKENPHVRYIEQTHTKLEHSVCANEMRKVSKSSTHPQHNPNEN
EILVADTYDQSQSPMAKAHGTSSYTPDKSSFNLATVVAETLGLGVQEESETQGPM
SPLGDELYHCLEGNHKKQPFE
SEQ ID NO. 15 - HE(3E) domain of human CtIP
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQAAELECEEDVIPDSPIT
AFSFSGVNRLRRKENPHVRYIEQTHTKLEHSVCANEMRKVSKSSTHPQHNPNENEI
LVADTYDQSQEPMAKAHGTSSYEPDKSSFNLATVVAETLGLGVQEESETQGPMEP
LGDELYHCLEGNHKKQPFE
SEQ ID NO. 16 - HE(3A) domain of human CtIP
MNISGSSCGSPNSADTSSDFKDLWTKLKECHDREVQGLQVKVTKLKQERILDAQR
LEEFFTKNQQLREQQKVLHETIKVLEDRLRAGLCDRCAVTEEHMRKKQQEFENIR
QQNLKLITELMNERNTLQEENKKLSEQLQQKIENDQQHQAAELECEEDVIPDSPIT
AFSFSGVNRLRRKENPHVRYIEQTHTKLEHSVCANEMRKVSKSSTHPQHNPNENEI
LVADTYDQSQAPMAKAHGTSSYAPDKSSFNLATVVAETLGLGVQEESETQGPMA
PLGDELYHCLEGNHKKQPFE
SEQ ID NO. 17 - SV40 NLS1
PKKKRKV
SEQ ID NO. 18 - NLS of nucleoplasmin
KRPAATKKAGQAKKKK
SEQ ID NO. 19 - NLS of c-Myc
PAAKRVKLD
SEQ ID NO. 20 - NLS of EGL-13
MSRRRKANPTKLSENAKKLAKEVEN
SEQ ID NO. 21 - Cas9-human CtIP
MDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVL
DATLIHQSITGLYETRIDLSQLGGDSRADPKKKRKVGSMNISGSSCGSPNSADTSSD
FKDLWTKLKECHDREVQGLQVKVTKLKQERILDAQRLEEFFTKNQQLREQQKVL
HETIKVLEDRLRAGLCDRCAVTEEHMRKKQQEFENIRQQNLKLITELMNERNTLQ
EENKKLSEQLQQKIENDQQHQAAELECEEDVIPDSPITAFSFSGVNRLRRKENPHV
RYIEQTHTKLEHSVCANEMRKVSKSSTHPQHNPNENEILVADTYDQSQSPMAKAH
GTSSYTPDKSSFNLATVVAETLGLGVQEESETQGPMSPLGDELYHCLEGNHKKQP
FEESTRNTEDSLRFSDSTSKTPPQEELPTRVSSPVFGATSSIKSGLDLNTSLSPSLLQP
GKKKHLKTLPFSNTCISRLEKTRSKSEDSALFTHHSLGSEVNKIIIQSSNKQILINKNI
SESLGEQNRTEYGKDSNTDKHLEPLKSLGGRTSKRKKTEEESEHEVSCPQASFDKE
NAFPFPMDNQFSMNGDCVMDKPLDLSDRFSAIQRQEKSQGSETSKNKFRQVTLYE
ALKTIPKGFSSSRKASDGNCTLPKDSPGEPCSQECIILQPLNKCSPDNKPSLQIKEEN
AVFKIPLRPRESLETENVLDDIKSAGSHEPIKIQTRSDHGGCELASVLQLNPCRTGKI
KSLQNNQDVSFENIQWSIDPGADLSQYKMDVTVIDTKDGSQSKLGGETVDMDCTL
VSETVLLKMKKQEQKGEKSSNEERKMNDSLEDMFDRTTHEEYESCLADSFSQAA
DEEEELSTATKKLHTHGDKQDKVKQKAFVEPYFKGDERETSLQNFPHIEVVRKKE
ERRKLLGHTCKECEIYYADMPAEEREKKLASCSRHRFRYIPPNTPENFWEVGFPST
QTCMERGYIKEDLDPCPRPKRRQPYNAIFSPKGKEQKT
SEQ ID NO. 22 - Cas9-HE domain of human CtIP
MDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVL
DATLIHQSITGLYETRIDLSQLGGDSRADPKKKRKVGSASMNISGSSCGSPNSADTS
SDFKDLWTKLKECHDREVQGLQVKVTKLKQERILDAQRLEEFFTKNQQLREQQK
VLHETIKVLEDRLRAGLCDRCAVTEEHMRKKQQEFENIRQQNLKLITELMNERNT
LQEENKKLSEQLQQKIENDQQHQAAELECEEDVIPDSPITAFSFSGVNRLRRKENPH
VRYIEQTHTKLEHSVCANEMRKVSKSSTHPQHNPNENEILVADTYDQSQSPMAKA
HGTSSYTPDKSSFNLATVVAETLGLGVQEESETQGPMSPLGDELYHCLEGNHKKQ
PFE
SEQ ID NO. 23 - Cas9-HE1 domain of human CtIP
MDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVL
DATLIHQSITGLYETRIDLSQLGGDSRADPKKKRKVGSASMNISGSSCGSPNSADTS
SDFKDLWTKLKECHDREVQGLQVKVTKLKQERILDAQRLEEFFTKNQQLREQQK
VLHETIKVLEDRLRAGLCDRCAVTEEHMRKKQQEFENIRQQNLKLITELMNERNT
LQEENKKLSEQLQQKIENDQQHQAAELECEEDVIPDSPITAFSF
SEQ ID NO. 24 - Cas9-HE(3E) domain of human CtIP
MDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVL
DATLIHQSITGLYETRIDLSQLGGDSRADPKKKRKVGSASMNISGSSCGSPNSADTS
SDFKDLWTKLKECHDREVQGLQVKVTKLKQERILDAQRLEEFFTKNQQLREQQK
VLHETIKVLEDRLRAGLCDRCAVTEEHMRKKQQEFENIRQQNLKLITELMNERNT
LQEENKKLSEQLQQKIENDQQHQAAELECEEDVIPDSPITAFSFSGVNRLRRKENPH
VRYIEQTHTKLEHSVCANEMRKVSKSSTHPQHNPNENEILVADTYDQSQEPMAKA
HGTSSYEPDKSSFNLATVVAETLGLGVQEESETQGPMEPLGDELYHCLEGNHKKQ
PFE
SEQ ID NO. 25 - Nucleic acid sequence of Cas9 of S. pyogenes
atggacaagaagtactccattgggctcgatatcggcacaaacagcgtcggctgggccgtcattacggacgagtacaaggtgccg
agcaaaaaattcaaagttctgggcaataccgatcgccacagcataaagaagaacctcattggcgccctcctgttcgactccgggga
gacggccgaagccacgcggctcaaaagaacagcacggcgcagatatacccgcagaaagaatcggatctgctacctgcaggag
atctttagtaatgagatggctaaggtggatgactctttcttccataggctggaggagtcctttttggtggaggaggataaaaagcacg
agcgccacccaatctttggcaatatcgtggacgaggtggcgtaccatgaaaagtacccaaccatatatcatctgaggaagaagctt
gtagacagtactgataaggctgacttgcggttgatctatctcgcgctggcgcatatgatcaaatttcggggacacttcctcatcgagg
gggacctgaacccagacaacagcgatgtcgacaaactctttatccaactggttcagacttacaatcagcttttcgaagagaacccga
tcaacgcatccggagttgacgccaaagcaatcctgagcgctaggctgtccaaatcccggcggctcgaaaacctcatcgcacagct
ccctggggagaagaagaacggcctgtttggtaatcttatcgccctgtcactcgggctgacccccaactttaaatctaacttcgacctg
gccgaagatgccaagcttcaactgagcaaagacacctacgatgatgatctcgacaatctgctggcccagatcggcgaccagtacg
cagacctttttttggcggcaaagaacctgtcagacgccattctgctgagtgatattctgcgagtgaacacggagatcaccaaagctc
cgctgagcgctagtatgatcaagcgctatgatgagcaccaccaagacttgactttgctgaaggcccttgtcagacagcaactgcct
gagaagtacaaggaaattttcttcgatcagtctaaaaatggctacgccggatacattgacggcggagcaagccaggaggaatttta
caaatttattaagcccatcttggaaaaaatggacggcaccgaggagctgctggtaaagcttaacagagaagatctgttgcgcaaac
agcgcactttcgacaatggaagcatcccccaccagattcacctgggcgaactgcacgctatcctcaggcggcaagaggatttcta
cccctttttgaaagataacagggaaaagattgagaaaatcctcacatttcggataccctactatgtaggccccctcgcccggggaaa
ttccagattcgcgtggatgactcgcaaatcagaagagaccatcactccctggaacttcgaggaagtcgtggataagggggcctctg
cccagtccttcatcgaaaggatgactaactttgataaaaatctgcctaacgaaaaggtgcttcctaaacactctctgctgtacgagtac
ttcacagtttataacgagctcaccaaggtcaaatacgtcacagaagggatgagaaagccagcattcctgtctggagagcagaaga
aagctatcgtggacctcctcttcaagacgaaccggaaagttaccgtgaaacagctcaaagaagactatttcaaaaagattgaatgttt
cgactctgttgaaatcagcggagtggaggatcgcttcaacgcatccctgggaacgtatcacgatctcctgaaaatcattaaagacaa
ggacttcctggacaatgaggagaacgaggacattcttgaggacattgtcctcacccttacgttgtttgaagatagggagatgattga
agaacgcttgaaaacttacgctcatctcttcgacgacaaagtcatgaaacagctcaagaggcgccgatatacaggatgggggcgg
ctgtcaagaaaactgatcaatgggatccgagacaagcagagtggaaagacaatcctggattttcttaagtccgatggatttgccaac
cggaacttcatgcagttgatccatgatgactctctcacctttaaggaggacatccagaaagcacaagtttctggccagggggacagt
cttcacgagcacatcgctaatcttgcaggtagcccagctatcaaaaagggaatactgcagaccgttaaggtcgtggatgaactcgt
caaagtaatgggaaggcataagcccgagaatatcgttatcgagatggcccgagagaaccaaactacccagaagggacagaaga
acagtagggaaaggatgaagaggattgaagagggtataaaagaactggggtcccaaatccttaaggaacacccagttgaaaaca
cccagcttcagaatgagaagctctacctgtactacctgcagaacggcagggacatgtacgtggatcaggaactggacatcaatcg
gctctccgactacgacgtggatcatatcgtgccccagtcttttctcaaagatgattctattgataataaagtgttgacaagatccgataa
aaatagagggaagagtgataacgtcccctcagaagaagttgtcaagaaaatgaaaaattattggcggcagctgctgaacgccaaa
ctgatcacacaacggaagttcgataatctgactaaggctgaacgaggtggcctgtctgagttggataaagccggcttcatcaaaag
gcagcttgttgagacacgccagatcaccaagcacgtggcccaaattctcgattcacgcatgaacaccaagtacgatgaaaatgac
aaactgattcgagaggtgaaagttattactctgaagtctaagctggtctcagatttcagaaaggactttcagttttataaggtgagaga
gatcaacaattaccaccatgcgcatgatgcctacctgaatgcagtggtaggcactgcacttatcaaaaaatatcccaagcttgaatct
gaatttgtttacggagactataaagtgtacgatgttaggaaaatgatcgcaaagtctgagcaggaaataggcaaggccaccgctaa
gtacttcttttacagcaatattatgaattttttcaagaccgagattacactggccaatggagagattcggaagcgaccacttatcgaaac
aaacggagaaacaggagaaatcgtgtgggacaagggtagggatttcgcgacagtccggaaggtcctgtccatgccgcaggtga
acatcgttaaaaagaccgaagtacagaccggaggcttctccaaggaaagtatcctcccgaaaaggaacagcgacaagctgatcg
cacgcaaaaaagattgggaccccaagaaatacggcggattcgattctcctacagtcgcttacagtgtactggttgtggccaaagtg
gagaaagggaagtctaaaaaactcaaaagcgtcaaggaactgctgggcatcacaatcatggagcgatcaagcttcgaaaaaaac
cccatcgactttctcgaggcgaaaggatataaagaggtcaaaaaagacctcatcattaagcttcccaagtactctctctttgagcttga
aaacggccggaaacgaatgctcgctagtgcgggcgagctgcagaaaggtaacgagctggcactgccctctaaatacgttaatttc
ttgtatctggccagccactatgaaaagctcaaagggtctcccgaagataatgagcagaagcagctgttcgtggaacaacacaaac
actaccttgatgagatcatcgagcaaataagcgaattctccaaaagagtgatcctcgccgacgctaacctcgataaggtgctttctgc
ttacaataagcacagggataagcccatcagggagcaggcagaaaacattatccacttgtttactctgaccaacttgggcgcgcctg
cagccttcaagtacttcgacaccaccatagacagaaagcggtacacctctacaaaggaggtcctggacgccacactgattcatca
gtcaattacggggctctatgaaacaagaatcgacctctctcagctcggtggagacagcagggctgac
SEQ ID NO. 26 - Nucleic acid sequence of human CtIP
atgaacatctcgggaagcagctgtggaagccctaactctgcagatacatctagtgactttaaggacctttggacaaaactaaaagaa
tgtcatgatagagaagtacaaggtttacaagtaaaagtaaccaagctaaaacaggaacgaatcttagatgcacaaagactagaaga
attcttcaccaaaaatcaacagctgagggaacagcagaaagtccttcatgaaaccattaaagttttagaagatcggttaagagcagg
cttatgtgatcgctgtgcagtaactgaagaacatatgcggaaaaaacagcaagagtttgaaaatatccggcagcagaatcttaaact
tattacagaacttatgaatgaaaggaatactctacaggaagaaaataaaaagctttctgaacaactccagcagaaaattgagaatgat
caacagcatcaagcagctgagcttgaatgtgaggaagacgttattccagattcaccgataacagccttctcattttctggcgttaacc
ggctacgaagaaaggagaacccccatgtccgatacatagaacaaacacatactaaattggagcactctgtgtgtgcaaatgaaatg
agaaaagtttccaagtcttcaactcatccacaacataatcctaatgaaaatgaaattctagtagctgacacttatgaccaaagtcaatct
ccaatggccaaagcacatggaacaagcagctatacccctgataagtcatcttttaatttagctacagttgttgctgaaacacttggact
tggtgttcaagaagaatctgaaactcaaggtcccatgagcccccttggtgatgagctctaccactgtctggaaggaaatcacaaga
aacagccttttgaggaatctacaagaaatactgaagatagtttaagattttcagattctacttcaaagactcctcctcaagaagaattac
ctactcgagtgtcatctcctgtatttggagctacctctagtatcaaaagtggtttagatttgaatacaagtttgtccccttctcttttacagc
ctgggaaaaaaaaacatctgaaaacactcccttttagcaacacttgtatatctagattagaaaaaactagatcaaaatctgaagatagt
gcccttttcacacatcacagtcttgggtctgaagtgaacaagatcattatccagtcatctaataaacagatacttataaataaaaatata
agtgaatccctaggtgaacagaataggactgagtacggtaaagattctaacactgataaacatttggagcccctgaaatcattggga
ggccgaacatccaaaaggaagaaaactgaggaagaaagtgaacatgaagtaagctgcccccaagcttcttttgataaagaaaatg
ctttcccttttccaatggataatcagttttccatgaatggagactgtgtgatggataaacctctggatctgtctgatcgattttcagctattc
agcgtcaagagaaaagccaaggaagtgagacttctaaaaacaaatttaggcaagtgactctttatgaggctttgaagaccattccaa
agggcttttcctcaagccgtaaggcctcagatggcaactgcacgttgcccaaagattccccaggggagccctgttcacaggaatg
catcatccttcagcccttgaataaatgctctccagacaataaaccatcattacaaataaaagaagaaaatgctgtctttaaaattcctct
acgtccacgtgaaagtttggagactgagaatgttttagatgacataaagagtgctggttctcatgagccaataaaaatacaaaccag
gtcagaccatggaggatgtgaacttgcatcagttcttcagttaaatccatgtagaactggtaaaataaagtctctacaaaacaaccaa
gatgtatcctttgaaaatatccagtggagtatagatccgggagcagacctttctcagtataaaatggatgttactgtaatagatacaaa
ggatggcagtcagtcaaaattaggaggagagacagtggacatggactgtacattggttagtgaaaccgttctcttaaaaatgaaga
agcaagagcagaagggagaaaaaagttcaaatgaagaaagaaaaatgaatgatagcttggaagatatgtttgatcggacaacac
atgaagagtatgaatcctgtttggcagacagtttctcccaagcagcagatgaagaggaggaattgtctactgccacaaagaaacta
cacactcatggtgataaacaagacaaagtcaagcagaaagcgtttgtggagccgtattttaaaggtgatgaaagagagactagctt
gcaaaattttcctcatattgaggtggttcggaaaaaagaggagagaagaaaactgcttgggcacacgtgtaaggaatgtgaaattta
ttatgcagatatgccagcagaagaaagagaaaagaaattggcttcctgctcaagacaccgattccgctacattccacccaacacac
cagagaatttttgggaagttggttttccttccactcagacttgtatggaaagaggttatattaaggaagatcttgatccttgtcctcgtcc
aaaaagacgtcagccttacaacgcaatattttctccaaaaggcaaggagcagaagacatag
SEQ ID NO. 27 - Nucleic acid sequence of HE domain of the human CtIP
atgaacatctcgggaagcagctgtggaagccctaactctgcagatacatctagtgactttaaggacctttggacaaaactaaaagaa
tgtcatgatagagaagtacaaggtttacaagtaaaagtaaccaagctaaaacaggaacgaatcttagatgcacaaagactagaaga
attcttcaccaaaaatcaacagctgagggaacagcagaaagtccttcatgaaaccattaaagttttagaagatcggttaagagcagg
cttatgtgatcgctgtgcagtaactgaagaacatatgcggaaaaaacagcaagagtttgaaaatatccggcagcagaatcttaaact
tattacagaacttatgaatgaaaggaatactctacaggaagaaaataaaaagctttctgaacaactccagcagaaaattgagaatgat
caacagcatcaagcagctgagcttgaatgtgaggaagacgttattccagattcaccgataacagccttctcattttctggcgttaacc
ggctacgaagaaaggagaacccccatgtccgatacatagaacaaacacatactaaattggagcactctgtgtgtgcaaatgaaatg
agaaaagtttccaagtcttcaactcatccacaacataatcctaatgaaaatgaaattctagtagctgacacttatgaccaaagtcaatct
ccaatggccaaagcacatggaacaagcagctatacccctgataagtcatcttttaatttagctacagttgttgctgaaacacttggact
tggtgttcaagaagaatctgaaactcaaggtcccatgagcccccttggtgatgagctctaccactgtctggaaggaaatcacaaga
aacagccttttgag
SEQ ID NO. 28 - Nucleic acid sequence of HE(3E) domain of the human CtIP
atgaacatctcgggaagcagctgtggaagccctaactctgcagatacatctagtgactttaaggacctttggacaaaactaaaagaa
tgtcatgatagagaagtacaaggtttacaagtaaaagtaaccaagctaaaacaggaacgaatcttagatgcacaaagactagaaga
attcttcaccaaaaatcaacagctgagggaacagcagaaagtccttcatgaaaccattaaagttttagaagatcggttaagagcagg
cttatgtgatcgctgtgcagtaactgaagaacatatgcggaaaaaacagcaagagtttgaaaatatccggcagcagaatcttaaact
tattacagaacttatgaatgaaaggaatactctacaggaagaaaataaaaagctttctgaacaactccagcagaaaattgagaatgat
caacagcatcaagcagctgagcttgaatgtgaggaagacgttattccagattcaccgataacagccttctcattttctggcgttaacc
ggctacgaagaaaggagaacccccatgtccgatacatagaacaaacacatactaaattggagcactctgtgtgtgcaaatgaaatg
agaaaagtttccaagtcttcaactcatccacaacataatcctaatgaaaatgaaattctagtagctgacacttatgaccaaagtcaaga
gccaatggccaaagcacatggaacaagcagctatgaacctgataagtcatcttttaatttagctacagttgttgctgaaacacttgga
cttggtgttcaagaagaatctgaaactcaaggtcccatggaaccccttggtgatgagctctaccactgtctggaaggaaatcacaag
aaacagccttttgag
SEQ ID NO. 29 - Nucleic acid of a Cas9-human CtIP fusion
gttgacattgattattgactagttattaatagtaatcaattacggggtcattagttcatagcccatatatggagttccgcgttacataactta
cggtaaatggcccgcctggctgaccgcccaacgacccccgcccattgacgtcaataatgacgtatgttcccatagtaacgccaata
gggactttccattgacgtcaatgggtggagtatttacggtaaactgcccacttggcagtacatcaagtgtatcatatgccaagtacgc
cccctattgacgtcaatgacggtaaatggcccgcctggcattatgcccagtacatgaccttatgggactttcctacttggcagtacatc
tacgtattagtcatcgctattaccatggtgatgcggttttggcagtacatcaatgggcgtggatagcggtttgactcacggggatttcc
aagtctccaccccattgacgtcaatgggagtttgttttggcaccaaaatcaacgggactttccaaaatgtcgtaacaactccgcccca
ttgacgcaaatgggcggtaggcgtgtacggtgggaggtctatataagcagagctcgtttagtgaaccgtcagatcgcctggagac
gccatccacgctgttttgacctccatagaagacaccgggaccgatccagcctccggactctagaggatcgaacccttgccaccatg
gacaagaagtactccattgggctcgatatcggcacaaacagcgtcggctgggccgtcattacggacgagtacaaggtgccgagc
aaaaaattcaaagttctgggcaataccgatcgccacagcataaagaagaacctcattggcgccctcctgttcgactccggggagac
ggccgaagccacgcggctcaaaagaacagcacggcgcagatatacccgcagaaagaatcggatctgctacctgcaggagatct
ttagtaatgagatggctaaggtggatgactctttcttccataggctggaggagtcctttttggtggaggaggataaaaagcacgagc
gccacccaatctttggcaatatcgtggacgaggtggcgtaccatgaaaagtacccaaccatatatcatctgaggaagaagcttgta
gacagtactgataaggctgacttgcggttgatctatctcgcgctggcgcatatgatcaaatttcggggacacttcctcatcgagggg
gacctgaacccagacaacagcgatgtcgacaaactctttatccaactggttcagacttacaatcagcttttcgaagagaacccgatc
aacgcatccggagttgacgccaaagcaatcctgagcgctaggctgtccaaatcccggcggctcgaaaacctcatcgcacagctc
cctggggagaagaagaacggcctgtttggtaatcttatcgccctgtcactcgggctgacccccaactttaaatctaacttcgacctgg
ccgaagatgccaagcttcaactgagcaaagacacctacgatgatgatctcgacaatctgctggcccagatcggcgaccagtacgc
agacctttttttggcggcaaagaacctgtcagacgccattctgctgagtgatattctgcgagtgaacacggagatcaccaaagctcc
gctgagcgctagtatgatcaagcgctatgatgagcaccaccaagacttgactttgctgaaggcccttgtcagacagcaactgcctg
agaagtacaaggaaattttcttcgatcagtctaaaaatggctacgccggatacattgacggcggagcaagccaggaggaattttac
aaatttattaagcccatcttggaaaaaatggacggcaccgaggagctgctggtaaagcttaacagagaagatctgttgcgcaaaca
gcgcactttcgacaatggaagcatcccccaccagattcacctgggcgaactgcacgctatcctcaggcggcaagaggatttctac
ccctttttgaaagataacagggaaaagattgagaaaatcctcacatttcggataccctactatgtaggccccctcgcccggggaaatt
ccagattcgcgtggatgactcgcaaatcagaagagaccatcactccctggaacttcgaggaagtcgtggataagggggcctctgc
ccagtccttcatcgaaaggatgactaactttgataaaaatctgcctaacgaaaaggtgcttcctaaacactctctgctgtacgagtact
tcacagtttataacgagctcaccaaggtcaaatacgtcacagaagggatgagaaagccagcattcctgtctggagagcagaagaa
agctatcgtggacctcctcttcaagacgaaccggaaagttaccgtgaaacagctcaaagaagactatttcaaaaagattgaatgtttc
gactctgttgaaatcagcggagtggaggatcgcttcaacgcatccctgggaacgtatcacgatctcctgaaaatcattaaagacaa
ggacttcctggacaatgaggagaacgaggacattcttgaggacattgtcctcacccttacgttgtttgaagatagggagatgattga
agaacgcttgaaaacttacgctcatctcttcgacgacaaagtcatgaaacagctcaagaggcgccgatatacaggatgggggcgg
ctgtcaagaaaactgatcaatgggatccgagacaagcagagtggaaagacaatcctggattttcttaagtccgatggatttgccaac
cggaacttcatgcagttgatccatgatgactctctcacctttaaggaggacatccagaaagcacaagtttctggccagggggacagt
cttcacgagcacatcgctaatcttgcaggtagcccagctatcaaaaagggaatactgcagaccgttaaggtcgtggatgaactcgt
caaagtaatgggaaggcataagcccgagaatatcgttatcgagatggcccgagagaaccaaactacccagaagggacagaaga
acagtagggaaaggatgaagaggattgaagagggtataaaagaactggggtcccaaatccttaaggaacacccagttgaaaaca
cccagcttcagaatgagaagctctacctgtactacctgcagaacggcagggacatgtacgtggatcaggaactggacatcaatcg
gctctccgactacgacgtggatcatatcgtgccccagtcttttctcaaagatgattctattgataataaagtgttgacaagatccgataa
aaatagagggaagagtgataacgtcccctcagaagaagttgtcaagaaaatgaaaaattattggcggcagctgctgaacgccaaa
ctgatcacacaacggaagttcgataatctgactaaggctgaacgaggtggcctgtctgagttggataaagccggcttcatcaaaag
gcagcttgttgagacacgccagatcaccaagcacgtggcccaaattctcgattcacgcatgaacaccaagtacgatgaaaatgac
aaactgattcgagaggtgaaagttattactctgaagtctaagctggtctcagatttcagaaaggactttcagttttataaggtgagaga
gatcaacaattaccaccatgcgcatgatgcctacctgaatgcagtggtaggcactgcacttatcaaaaaatatcccaagcttgaatct
gaatttgtttacggagactataaagtgtacgatgttaggaaaatgatcgcaaagtctgagcaggaaataggcaaggccaccgctaa
gtacttcttttacagcaatattatgaattttttcaagaccgagattacactggccaatggagagattcggaagcgaccacttatcgaaac
aaacggagaaacaggagaaatcgtgtgggacaagggtagggatttcgcgacagtccggaaggtcctgtccatgccgcaggtga
acatcgttaaaaagaccgaagtacagaccggaggcttctccaaggaaagtatcctcccgaaaaggaacagcgacaagctgatcg
cacgcaaaaaagattgggaccccaagaaatacggcggattcgattctcctacagtcgcttacagtgtactggttgtggccaaagtg
gagaaagggaagtctaaaaaactcaaaagcgtcaaggaactgctgggcatcacaatcatggagcgatcaagcttcgaaaaaaac
cccatcgactttctcgaggcgaaaggatataaagaggtcaaaaaagacctcatcattaagcttcccaagtactctctctttgagcttga
aaacggccggaaacgaatgctcgctagtgcgggcgagctgcagaaaggtaacgagctggcactgccctctaaatacgttaatttc
ttgtatctggccagccactatgaaaagctcaaagggtctcccgaagataatgagcagaagcagctgttcgtggaacaacacaaac
actaccttgatgagatcatcgagcaaataagcgaattctccaaaagagtgatcctcgccgacgctaacctcgataaggtgctttctgc
ttacaataagcacagggataagcccatcagggagcaggcagaaaacattatccacttgtttactctgaccaacttgggcgcgcctg
cagccttcaagtacttcgacaccaccatagacagaaagcggtacacctctacaaaggaggtcctggacgccacactgattcatca
gtcaattacggggctctatgaaacaagaatcgacctctctcagctcggtggagacagcagggctgaccccaagaagaagaggaa
ggtgggatccatgaacatctcgggaagcagctgtggaagccctaactctgcagatacatctagtgactttaaggacctttggacaaa
actaaaagaatgtcatgatagagaagtacaaggtttacaagtaaaagtaaccaagctaaaacaggaacgaatcttagatgcacaaa
gactagaagaattcttcaccaaaaatcaacagctgagggaacagcagaaagtccttcatgaaaccattaaagttttagaagatcggt
taagagcaggcttatgtgatcgctgtgcagtaactgaagaacatatgcggaaaaaacagcaagagtttgaaaatatccggcagca
gaatcttaaacttattacagaacttatgaatgaaaggaatactctacaggaagaaaataaaaagctttctgaacaactccagcagaaa
attgagaatgatcaacagcatcaagcagctgagcttgaatgtgaggaagacgttattccagattcaccgataacagccttctcattttc
tggcgttaaccggctacgaagaaaggagaacccccatgtccgatacatagaacaaacacatactaaattggagcactctgtgtgtg
caaatgaaatgagaaaagtttccaagtcttcaactcatccacaacataatcctaatgaaaatgaaattctagtagctgacacttatgac
caaagtcaatctccaatggccaaagcacatggaacaagcagctatacccctgataagtcatcttttaatttagctacagttgttgctga
aacacttggacttggtgttcaagaagaatctgaaactcaaggtcccatgagcccccttggtgatgagctctaccactgtctggaagg
aaatcacaagaaacagccttttgaggaatctacaagaaatactgaagatagtttaagattttcagattctacttcaaagactcctcctca
agaagaattacctactcgagtgtcatctcctgtatttggagctacctctagtatcaaaagtggtttagatttgaatacaagtttgtcccctt
ctcttttacagcctgggaaaaaaaaacatctgaaaacactcccttttagcaacacttgtatatctagattagaaaaaactagatcaaaat
ctgaagatagtgcccttttcacacatcacagtcttgggtctgaagtgaacaagatcattatccagtcatctaataaacagatacttataa
ataaaaatataagtgaatccctaggtgaacagaataggactgagtacggtaaagattctaacactgataaacatttggagcccctga
aatcattgggaggccgaacatccaaaaggaagaaaactgaggaagaaagtgaacatgaagtaagctgcccccaagcttcttttga
taaagaaaatgctttcccttttccaatggataatcagttttccatgaatggagactgtgtgatggataaacctctggatctgtctgatcga
ttttcagctattcagcgtcaagagaaaagccaaggaagtgagacttctaaaaacaaatttaggcaagtgactctttatgaggctttgaa
gaccattccaaagggcttttcctcaagccgtaaggcctcagatggcaactgcacgttgcccaaagattccccaggggagccctgtt
cacaggaatgcatcatccttcagcccttgaataaatgctctccagacaataaaccatcattacaaataaaagaagaaaatgctgtcttt
aaaattcctctacgtccacgtgaaagtttggagactgagaatgttttagatgacataaagagtgctggttctcatgagccaataaaaat
acaaaccaggtcagaccatggaggatgtgaacttgcatcagttcttcagttaaatccatgtagaactggtaaaataaagtctctacaa
aacaaccaagatgtatcctttgaaaatatccagtggagtatagatccgggagcagacctttctcagtataaaatggatgttactgtaat
agatacaaaggatggcagtcagtcaaaattaggaggagagacagtggacatggactgtacattggttagtgaaaccgttctcttaa
aaatgaagaagcaagagcagaagggagaaaaaagttcaaatgaagaaagaaaaatgaatgatagcttggaagatatgtttgatcg
gacaacacatgaagagtatgaatcctgtttggcagacagtttctcccaagcagcagatgaagaggaggaattgtctactgccacaa
agaaactacacactcatggtgataaacaagacaaagtcaagcagaaagcgtttgtggagccgtattttaaaggtgatgaaagagag
actagcttgcaaaattttcctcatattgaggtggttcggaaaaaagaggagagaagaaaactgcttgggcacacgtgtaaggaatgt
gaaatttattatgcagatatgccagcagaagaaagagaaaagaaattggcttcctgctcaagacaccgattccgctacattccaccc
aacacaccagagaatttttgggaagttggttttccttccactcagacttgtatggaaagaggttatattaaggaagatcttgatccttgtc
ctcgtccaaaaagacgtcagccttacaacgcaatattttctccaaaaggcaaggagcagaagacatagaccggttagtaatgagttt
aaacgggggaggctaactgaaacacggaaggagacaataccggaaggaacccgcgctatgacggcaataaaaagacagaata
aaacgcacgggtgttgggtcgtttgttcataaacgcggggttcggtcccagggctggcactctgtcgataccccaccgagacccc
attggggccaatacgcccgcgtttcttccttttccccaccccaccccccaagttcgggtgaaggcccagggctcgcagccaacgtc
ggggcggcaggccctgccatagc
SEQ ID NO. 30 - Nucleic acid of a Cas9-HE domain of the human CtIP fusion
gttgacattgattattgactagttattaatagtaatcaattacggggtcattagttcatagcccatatatggagttccgcgttacataactta
cggtaaatggcccgcctggctgaccgcccaacgacccccgcccattgacgtcaataatgacgtatgttcccatagtaacgccaata
gggactttccattgacgtcaatgggtggagtatttacggtaaactgcccacttggcagtacatcaagtgtatcatatgccaagtacgc
cccctattgacgtcaatgacggtaaatggcccgcctggcattatgcccagtacatgaccttatgggactttcctacttggcagtacatc
tacgtattagtcatcgctattaccatggtgatgcggttttggcagtacatcaatgggcgtggatagcggtttgactcacggggatttcc
aagtctccaccccattgacgtcaatgggagtttgttttggcaccaaaatcaacgggactttccaaaatgtcgtaacaactccgcccca
ttgacgcaaatgggcggtaggcgtgtacggtgggaggtctatataagcagagctcgtttagtgaaccgtcagatcgcctggagac
gccatccacgctgttttgacctccatagaagacaccgggaccgatccagcctccggactctagaggatcgaacccttgccaccatg
gacaagaagtactccattgggctcgatatcggcacaaacagcgtcggctgggccgtcattacggacgagtacaaggtgccgagc
aaaaaattcaaagttctgggcaataccgatcgccacagcataaagaagaacctcattggcgccctcctgttcgactccggggagac
ggccgaagccacgcggctcaaaagaacagcacggcgcagatatacccgcagaaagaatcggatctgctacctgcaggagatct
ttagtaatgagatggctaaggtggatgactctttcttccataggctggaggagtcctttttggtggaggaggataaaaagcacgagc
gccacccaatctttggcaatatcgtggacgaggtggcgtaccatgaaaagtacccaaccatatatcatctgaggaagaagcttgta
gacagtactgataaggctgacttgcggttgatctatctcgcgctggcgcatatgatcaaatttcggggacacttcctcatcgagggg
gacctgaacccagacaacagcgatgtcgacaaactctttatccaactggttcagacttacaatcagcttttcgaagagaacccgatc
aacgcatccggagttgacgccaaagcaatcctgagcgctaggctgtccaaatcccggcggctcgaaaacctcatcgcacagctc
cctggggagaagaagaacggcctgtttggtaatcttatcgccctgtcactcgggctgacccccaactttaaatctaacttcgacctgg
ccgaagatgccaagcttcaactgagcaaagacacctacgatgatgatctcgacaatctgctggcccagatcggcgaccagtacgc
agacctttttttggcggcaaagaacctgtcagacgccattctgctgagtgatattctgcgagtgaacacggagatcaccaaagctcc
gctgagcgctagtatgatcaagcgctatgatgagcaccaccaagacttgactttgctgaaggcccttgtcagacagcaactgcctg
agaagtacaaggaaattttcttcgatcagtctaaaaatggctacgccggatacattgacggcggagcaagccaggaggaattttac
aaatttattaagcccatcttggaaaaaatggacggcaccgaggagctgctggtaaagcttaacagagaagatctgttgcgcaaaca
gcgcactttcgacaatggaagcatcccccaccagattcacctgggcgaactgcacgctatcctcaggcggcaagaggatttctac
ccctttttgaaagataacagggaaaagattgagaaaatcctcacatttcggataccctactatgtaggccccctcgcccggggaaatt
ccagattcgcgtggatgactcgcaaatcagaagagaccatcactccctggaacttcgaggaagtcgtggataagggggcctctgc
ccagtccttcatcgaaaggatgactaactttgataaaaatctgcctaacgaaaaggtgcttcctaaacactctctgctgtacgagtact
tcacagtttataacgagctcaccaaggtcaaatacgtcacagaagggatgagaaagccagcattcctgtctggagagcagaagaa
agctatcgtggacctcctcttcaagacgaaccggaaagttaccgtgaaacagctcaaagaagactatttcaaaaagattgaatgtttc
gactctgttgaaatcagcggagtggaggatcgcttcaacgcatccctgggaacgtatcacgatctcctgaaaatcattaaagacaa
ggacttcctggacaatgaggagaacgaggacattcttgaggacattgtcctcacccttacgttgtttgaagatagggagatgattga
agaacgcttgaaaacttacgctcatctcttcgacgacaaagtcatgaaacagctcaagaggcgccgatatacaggatgggggcgg
ctgtcaagaaaactgatcaatgggatccgagacaagcagagtggaaagacaatcctggattttcttaagtccgatggatttgccaac
cggaacttcatgcagttgatccatgatgactctctcacctttaaggaggacatccagaaagcacaagtttctggccagggggacagt
cttcacgagcacatcgctaatcttgcaggtagcccagctatcaaaaagggaatactgcagaccgttaaggtcgtggatgaactcgt
caaagtaatgggaaggcataagcccgagaatatcgttatcgagatggcccgagagaaccaaactacccagaagggacagaaga
acagtagggaaaggatgaagaggattgaagagggtataaaagaactggggtcccaaatccttaaggaacacccagttgaaaaca
cccagcttcagaatgagaagctctacctgtactacctgcagaacggcagggacatgtacgtggatcaggaactggacatcaatcg
gctctccgactacgacgtggatcatatcgtgccccagtcttttctcaaagatgattctattgataataaagtgttgacaagatccgataa
aaatagagggaagagtgataacgtcccctcagaagaagttgtcaagaaaatgaaaaattattggcggcagctgctgaacgccaaa
ctgatcacacaacggaagttcgataatctgactaaggctgaacgaggtggcctgtctgagttggataaagccggcttcatcaaaag
gcagcttgttgagacacgccagatcaccaagcacgtggcccaaattctcgattcacgcatgaacaccaagtacgatgaaaatgac
aaactgattcgagaggtgaaagttattactctgaagtctaagctggtctcagatttcagaaaggactttcagttttataaggtgagaga
gatcaacaattaccaccatgcgcatgatgcctacctgaatgcagtggtaggcactgcacttatcaaaaaatatcccaagcttgaatct
gaatttgtttacggagactataaagtgtacgatgttaggaaaatgatcgcaaagtctgagcaggaaataggcaaggccaccgctaa
gtacttcttttacagcaatattatgaattttttcaagaccgagattacactggccaatggagagattcggaagcgaccacttatcgaaac
aaacggagaaacaggagaaatcgtgtgggacaagggtagggatttcgcgacagtccggaaggtcctgtccatgccgcaggtga
acatcgttaaaaagaccgaagtacagaccggaggcttctccaaggaaagtatcctcccgaaaaggaacagcgacaagctgatcg
cacgcaaaaaagattgggaccccaagaaatacggcggattcgattctcctacagtcgcttacagtgtactggttgtggccaaagtg
gagaaagggaagtctaaaaaactcaaaagcgtcaaggaactgctgggcatcacaatcatggagcgatcaagcttcgaaaaaaac
cccatcgactttctcgaggcgaaaggatataaagaggtcaaaaaagacctcatcattaagcttcccaagtactctctctttgagcttga
aaacggccggaaacgaatgctcgctagtgcgggcgagctgcagaaaggtaacgagctggcactgccctctaaatacgttaatttc
ttgtatctggccagccactatgaaaagctcaaagggtctcccgaagataatgagcagaagcagctgttcgtggaacaacacaaac
actaccttgatgagatcatcgagcaaataagcgaattctccaaaagagtgatcctcgccgacgctaacctcgataaggtgctttctgc
ttacaataagcacagggataagcccatcagggagcaggcagaaaacattatccacttgtttactctgaccaacttgggcgcgcctg
cagccttcaagtacttcgacaccaccatagacagaaagcggtacacctctacaaaggaggtcctggacgccacactgattcatca
gtcaattacggggctctatgaaacaagaatcgacctctctcagctcggtggagacagcagggctgaccccaagaagaagaggaa
ggtgggatccgctagcatgaacatctcgggaagcagctgtggaagccctaactctgcagatacatctagtgactttaaggacctttg
gacaaaactaaaagaatgtcatgatagagaagtacaaggtttacaagtaaaagtaaccaagctaaaacaggaacgaatcttagatg
cacaaagactagaagaattcttcaccaaaaatcaacagctgagggaacagcagaaagtccttcatgaaaccattaaagttttagaa
gatcggttaagagcaggcttatgtgatcgctgtgcagtaactgaagaacatatgcggaaaaaacagcaagagtttgaaaatatccg
gcagcagaatcttaaacttattacagaacttatgaatgaaaggaatactctacaggaagaaaataaaaagctttctgaacaactccag
cagaaaattgagaatgatcaacagcatcaagcagctgagcttgaatgtgaggaagacgttattccagattcaccgataacagccttc
tcattttctggcgttaaccggctacgaagaaaggagaacccccatgtccgatacatagaacaaacacatactaaattggagcactct
gtgtgtgcaaatgaaatgagaaaagtttccaagtcttcaactcatccacaacataatcctaatgaaaatgaaattctagtagctgacac
ttatgaccaaagtcaatctccaatggccaaagcacatggaacaagcagctatacccctgataagtcatcttttaatttagctacagttgt
tgctgaaacacttggacttggtgttcaagaagaatctgaaactcaaggtcccatgagcccccttggtgatgagctctaccactgtctg
gaaggaaatcacaagaaacagccttttgagtagaccggttagtaatgagtttaaacgggggaggctaactgaaacacggaagga
gacaataccggaaggaacccgcgctatgacggcaataaaaagacagaataaaacgcacgggtgttgggtcgtttgttcataaacg
cggggttcggtcccagggctggcactctgtcgataccccaccgagaccccattggggccaatacgcccgcgtttcttccttttcccc
accccaccccccaagttcgggtgaaggcccagggctcgcagccaacgtcggggcggcaggccctgccatagc
SEQ ID NO. 31 - Nucleic acid of a Cas9-HE(3E) domain of the human CtIP fusion
gttgacattgattattgactagttattaatagtaatcaattacggggtcattagttcatagcccatatatggagttccgcgttacataactta
cggtaaatggcccgcctggctgaccgcccaacgacccccgcccattgacgtcaataatgacgtatgttcccatagtaacgccaata
gggactttccattgacgtcaatgggtggagtatttacggtaaactgcccacttggcagtacatcaagtgtatcatatgccaagtacgc
cccctattgacgtcaatgacggtaaatggcccgcctggcattatgcccagtacatgaccttatgggactttcctacttggcagtacatc
tacgtattagtcatcgctattaccatggtgatgcggttttggcagtacatcaatgggcgtggatagcggtttgactcacggggatttcc
aagtctccaccccattgacgtcaatgggagtttgttttggcaccaaaatcaacgggactttccaaaatgtcgtaacaactccgcccca
ttgacgcaaatgggcggtaggcgtgtacggtgggaggtctatataagcagagctcgtttagtgaaccgtcagatcgcctggagac
gccatccacgctgttttgacctccatagaagacaccgggaccgatccagcctccggactctagaggatcgaacccttgccaccatg
gacaagaagtactccattgggctcgatatcggcacaaacagcgtcggctgggccgtcattacggacgagtacaaggtgccgagc
aaaaaattcaaagttctgggcaataccgatcgccacagcataaagaagaacctcattggcgccctcctgttcgactccggggagac
ggccgaagccacgcggctcaaaagaacagcacggcgcagatatacccgcagaaagaatcggatctgctacctgcaggagatct
ttagtaatgagatggctaaggtggatgactctttcttccataggctggaggagtcctttttggtggaggaggataaaaagcacgagc
gccacccaatctttggcaatatcgtggacgaggtggcgtaccatgaaaagtacccaaccatatatcatctgaggaagaagcttgta
gacagtactgataaggctgacttgcggttgatctatctcgcgctggcgcatatgatcaaatttcggggacacttcctcatcgagggg
gacctgaacccagacaacagcgatgtcgacaaactctttatccaactggttcagacttacaatcagcttttcgaagagaacccgatc
aacgcatccggagttgacgccaaagcaatcctgagcgctaggctgtccaaatcccggcggctcgaaaacctcatcgcacagctc
cctggggagaagaagaacggcctgtttggtaatcttatcgccctgtcactcgggctgacccccaactttaaatctaacttcgacctgg
ccgaagatgccaagcttcaactgagcaaagacacctacgatgatgatctcgacaatctgctggcccagatcggcgaccagtacgc
agacctttttttggcggcaaagaacctgtcagacgccattctgctgagtgatattctgcgagtgaacacggagatcaccaaagctcc
gctgagcgctagtatgatcaagcgctatgatgagcaccaccaagacttgactttgctgaaggcccttgtcagacagcaactgcctg
agaagtacaaggaaattttcttcgatcagtctaaaaatggctacgccggatacattgacggcggagcaagccaggaggaattttac
aaatttattaagcccatcttggaaaaaatggacggcaccgaggagctgctggtaaagcttaacagagaagatctgttgcgcaaaca
gcgcactttcgacaatggaagcatcccccaccagattcacctgggcgaactgcacgctatcctcaggcggcaagaggatttctac
ccctttttgaaagataacagggaaaagattgagaaaatcctcacatttcggataccctactatgtaggccccctcgcccggggaaatt
ccagattcgcgtggatgactcgcaaatcagaagagaccatcactccctggaacttcgaggaagtcgtggataagggggcctctgc
ccagtccttcatcgaaaggatgactaactttgataaaaatctgcctaacgaaaaggtgcttcctaaacactctctgctgtacgagtact
tcacagtttataacgagctcaccaaggtcaaatacgtcacagaagggatgagaaagccagcattcctgtctggagagcagaagaa
agctatcgtggacctcctcttcaagacgaaccggaaagttaccgtgaaacagctcaaagaagactatttcaaaaagattgaatgtttc
gactctgttgaaatcagcggagtggaggatcgcttcaacgcatccctgggaacgtatcacgatctcctgaaaatcattaaagacaa
ggacttcctggacaatgaggagaacgaggacattcttgaggacattgtcctcacccttacgttgtttgaagatagggagatgattga
agaacgcttgaaaacttacgctcatctcttcgacgacaaagtcatgaaacagctcaagaggcgccgatatacaggatgggggcgg
ctgtcaagaaaactgatcaatgggatccgagacaagcagagtggaaagacaatcctggattttcttaagtccgatggatttgccaac
cggaacttcatgcagttgatccatgatgactctctcacctttaaggaggacatccagaaagcacaagtttctggccagggggacagt
cttcacgagcacatcgctaatcttgcaggtagcccagctatcaaaaagggaatactgcagaccgttaaggtcgtggatgaactcgt
caaagtaatgggaaggcataagcccgagaatatcgttatcgagatggcccgagagaaccaaactacccagaagggacagaaga
acagtagggaaaggatgaagaggattgaagagggtataaaagaactggggtcccaaatccttaaggaacacccagttgaaaaca
cccagcttcagaatgagaagctctacctgtactacctgcagaacggcagggacatgtacgtggatcaggaactggacatcaatcg
gctctccgactacgacgtggatcatatcgtgccccagtcttttctcaaagatgattctattgataataaagtgttgacaagatccgataa
aaatagagggaagagtgataacgtcccctcagaagaagttgtcaagaaaatgaaaaattattggcggcagctgctgaacgccaaa
ctgatcacacaacggaagttcgataatctgactaaggctgaacgaggtggcctgtctgagttggataaagccggcttcatcaaaag
gcagcttgttgagacacgccagatcaccaagcacgtggcccaaattctcgattcacgcatgaacaccaagtacgatgaaaatgac
aaactgattcgagaggtgaaagttattactctgaagtctaagctggtctcagatttcagaaaggactttcagttttataaggtgagaga
gatcaacaattaccaccatgcgcatgatgcctacctgaatgcagtggtaggcactgcacttatcaaaaaatatcccaagcttgaatct
gaatttgtttacggagactataaagtgtacgatgttaggaaaatgatcgcaaagtctgagcaggaaataggcaaggccaccgctaa
gtacttcttttacagcaatattatgaattttttcaagaccgagattacactggccaatggagagattcggaagcgaccacttatcgaaac
aaacggagaaacaggagaaatcgtgtgggacaagggtagggatttcgcgacagtccggaaggtcctgtccatgccgcaggtga
acatcgttaaaaagaccgaagtacagaccggaggcttctccaaggaaagtatcctcccgaaaaggaacagcgacaagctgatcg
cacgcaaaaaagattgggaccccaagaaatacggcggattcgattctcctacagtcgcttacagtgtactggttgtggccaaagtg
gagaaagggaagtctaaaaaactcaaaagcgtcaaggaactgctgggcatcacaatcatggagcgatcaagcttcgaaaaaaac
cccatcgactttctcgaggcgaaaggatataaagaggtcaaaaaagacctcatcattaagcttcccaagtactctctctttgagcttga
aaacggccggaaacgaatgctcgctagtgcgggcgagctgcagaaaggtaacgagctggcactgccctctaaatacgttaatttc
ttgtatctggccagccactatgaaaagctcaaagggtctcccgaagataatgagcagaagcagctgttcgtggaacaacacaaac
actaccttgatgagatcatcgagcaaataagcgaattctccaaaagagtgatcctcgccgacgctaacctcgataaggtgctttctgc
ttacaataagcacagggataagcccatcagggagcaggcagaaaacattatccacttgtttactctgaccaacttgggcgcgcctg
cagccttcaagtacttcgacaccaccatagacagaaagcggtacacctctacaaaggaggtcctggacgccacactgattcatca
gtcaattacggggctctatgaaacaagaatcgacctctctcagctcggtggagacagcagggctgaccccaagaagaagaggaa
ggtgggatccgctagcatgaacatctcgggaagcagctgtggaagccctaactctgcagatacatctagtgactttaaggacctttg
gacaaaactaaaagaatgtcatgatagagaagtacaaggtttacaagtaaaagtaaccaagctaaaacaggaacgaatcttagatg
cacaaagactagaagaattcttcaccaaaaatcaacagctgagggaacagcagaaagtccttcatgaaaccattaaagttttagaa
gatcggttaagagcaggcttatgtgatcgctgtgcagtaactgaagaacatatgcggaaaaaacagcaagagtttgaaaatatccg
gcagcagaatcttaaacttattacagaacttatgaatgaaaggaatactctacaggaagaaaataaaaagctttctgaacaactccag
cagaaaattgagaatgatcaacagcatcaagcagctgagcttgaatgtgaggaagacgttattccagattcaccgataacagccttc
tcattttctggcgttaaccggctacgaagaaaggagaacccccatgtccgatacatagaacaaacacatactaaattggagcactct
gtgtgtgcaaatgaaatgagaaaagtttccaagtcttcaactcatccacaacataatcctaatgaaaatgaaattctagtagctgacac
ttatgaccaaagtcaagagccaatggccaaagcacatggaacaagcagctatgaacctgataagtcatcttttaatttagctacagtt
gttgctgaaacacttggacttggtgttcaagaagaatctgaaactcaaggtcccatggaaccccttggtgatgagctctaccactgtc
tggaaggaaatcacaagaaacagccttttgagtagaccggttagtaatgagtttaaacgggggaggctaactgaaacacggaagg
agacaataccggaaggaacccgcgctatgacggcaataaaaagacagaataaaacgcacgggtgttgggtcgtttgttcataaac
gcggggttcggtcccagggctggcactctgtcgataccccaccgagaccccattggggccaatacgcccgcgtttcttccttttccc
caccccaccccccaagttcgggtgaaggcccagggctcgcagccaacgtcggggcggcaggccctgccatagc
SEQ ID NO. 32 - T7AAVFw (primer)
cagcaccaggatcagtgaaa
SEQ ID NO. 33 - T7AAVRev (primer)
ctatgtccacttcaggacagca
SEQ ID NO. 34- rROSA-5HAFor (primer)
ttcttccacttgcgatccttg
SEQ ID NO. 35 - 5CAGpRev (primer)
ggctatgaactaatgaccccgtaat
SEQ ID NO. 36 - 3BGHpA-Up2 (primer)
ccagatttttcctcctctcctg
SEQ ID NO. 37 - rROSAfw1 (primer)
tgaactgtgaataggcccaagtg
SEQ ID NO. 38 - rROSA26-5outFor (primer)
tcccaccctccccttcctct
SEQ ID NO. 39 - 5CAGpRev (primer)
ggctatgaactaatgaccccgtaat
SEQ ID NO. 40 - 3BGHpA-Up2 (primer)
ccagatttttcctcctctcctg
SEQ ID NO. 41 - rROSA26-3outRev (primer)
tgggtatcactggctgtcctagata
SEQ ID NO. 42 - rROSArev1 (primer)
gcattttaaaagagcccagtacttca
SEQ ID NO. 43 - Nucleic acid sequence of the tetramerization domain of human CtIP
gacctttggacaaaactaaaagaatgtcatgatagagaagtacaaggtttacaagtaaaagtaaccaagcta
SEQ ID NO. 44 - Nucleic acid sequence of the dimerization domain of human CtIP
aaacaggaacgaatcttagatgcacaaagactagaagaattcttcaccaaaaatcaacagctgagggaacagcagaaagtccttc
atgaaaccattaaagttttagaagatcggttaagagcaggcttatgtgatcgctgtgcagtaactgaagaacatatgcggaaaaaac
agcaagagtttgaaaatatccggcagcagaatcttaaacttattacagaacttatgaatgaaaggaatactctacaggaagaaaata
aaaagctttctgaacaactccagcagaaaattgagaatgatcaacagcatcaagcagctgagcttgaatgtgaggaagacgttattc
cagattcaccgataaca
SEQ ID NO. 45 - Nucleic acid sequence of the HE3 domain of human CtIP
acagccttctcattttctggcgttaaccggctacgaagaaaggagaacccccatgtccgatacatagaacaaacacatactaaattg
gagcactctgtgtgtgcaaatgaaatgagaaaagtttccaagtcttcaactcatccacaacataatcctaatgaaaatgaaattctagt
agctgacacttatgaccaaagtcaatctccaatggccaaagcacatggaacaagcagctatacccctgataagtcatcttttaattta
gctacagttgttgctgaaacacttggacttggtgttcaagaagaatctgaaactcaaggtcccatgagcccccttggtgatgagctct
accactgtctggaaggaaatcacaagaaacagccttttgag
SEQ ID NO. 46 - Nucleic acid sequence of the gRNA for targeting the AAVS1 safe
harbor locus
ggggccactagggacaggatgttttagagctagaaatagcaagttaaaataaggctagtccgttatcaacttgaaaaagtggcacc
gagtcggtgc
SEQ ID NO. 47 - Nucleic acid sequence of the rat ROSA donor sequence
gtaacccctagggagttggggctcagtcgggttgtattggagacaagaagcacttgctctccaaaagtcggtttgagttatcattaag
ggagctgcagtggagtaggcggagaaaaggccgcacccttctcaggacgggggaggggagtgttgcaatacctttctgggagtt
ctctgctgcctcctgtcttctgaggaccgccctgggcctggaagattcccttcccccttcttccctcgtgatctgcaactggagtctttc
tggaagataggcgggagtcttctgggcaggcttaaaggctaacctggtgcgtggggcgttgtcctgcagaggaattgaacaggtg
taaaattggaggggcaagacttcccacagattttcgattgtgttgttaagtattgtaataggggcaaataagggaaatagactaggca
ctcacctggggttttatgcagcaaaactacaggttattattgcttgtgatccgccctggagaatttttcaccgaggtagattgaagacat
gcccacccaaattttaatattcttccacttgcgatccttgctacagtatgaaattacagtatcgtgaattagaatatataagcagaatttta
agcattttaaaagagcccagtacttcatgtctgtctctcccacttctgcagccctatcaaagggtattttagcacactcattttagtcccat
tttcatttgttgtactggcttatccaatccctagacagagcactggcattccctctctcctgatcttagaagtccgatgactcatgaaacc
agacagattagtgtcgacattgattattgactagttattaatagtaatcaattacggggtcattagttcatagcccatatatggagttccg
cgttacataacttacggtaaatggcccgcctggctgaccgcccaacgacccccgcccattgacgtcaataatgacgtatgttcccat
agtaacgccaatagggactttccattgacgtcaatgggtggactatttacggtaaactgcccacttggcagtacatcaagtgtatcat
atgccaagtacgccccctattgacgtcaatgacggtaaatggcccgcctggcattatgcccagtacatgaccttatgggactttccta
cttggcagtacatctacgtattagtcatcgctattaccatgggtcgaggtgagccccacgttctgcttcactctccccatctccccccc
ctccccacccccaattttgtatttatttattttttaattattttgtgcagcgatgggggcggggggggggggggcgcgcgccaggcgg
ggcggggcggggcgaggggcggggcggggcgaggcggagaggtgcggcggcagccaatcagagcggcgcgctccgaaa
gtttccttttatggcgaggcggcggcggcggcggccctataaaaagcgaagcgcgcggcgggcgggagtcgctgcgttgccttc
gccccgtgccccgctccgcgccgcctcgcgccgcccgccccggctctgactgaccgcgttactcccacaggtgagcgggcgg
gacggcccttctcctccgggctgtaattagcgcttggtttaatgacggctcgtttcttttctgtggctgcgtgaaagccttaaagggctc
cgggagggccctttgtgcgggggggagcggctcggggggtgcgtgcgtgtgtgtgtgcgtggggagcgccgcgtgcggccc
gcgctgcccggcggctgtgagcgctgcgggcgcggcgcggggctttgtgcgctccgcgtgtgcgcgaggggagcgcggccg
ggggcggtgccccgcggtgcgggggggctgcgaggggaacaaaggctgcgtgcggggtgtgtgcgtgggggggtgagcag
ggggtgtgggcgcggcggtcgggctgtaacccccccctgcacccccctccccgagttgctgagcacggcccggcttcgggtgc
ggggctccgtgcggggcgtggcgcggggctcgccgtgccgggcggggggtggcggcaggtgggggtgccgggcggggcg
gggccgcctcgggccggggagggctcgggggaggggcgcggcggccccggagcgccggcggctgtcgaggcgcggcga
gccgcagccattgccttttatggtaatcgtgcgagagggcgcagggacttcctttgtcccaaatctggcggagccgaaatctggga
ggcgccgccgcaccccctctagcgggcgcgggcgaagcggtgcggcgccggcaggaaggaaatgggcggggagggccttc
gtgcgtcgccgcgccgccgtccccttctccatctccagcctcggggctgccgcagggggacggctgccttcgggggggacggg
gcagggcggggttcggcttctggcgtgtgaccggcggctctagagcctctgctaaccatgttcatgccttcttctttttcctacagctc
ctgggcaacgtgctggttgttgtgctgtctcatcattttggcaaagaattgattaattcgagcgaacgcgtcgagtcgctcggtacgat
ttgtaatttgatccaccggtcgccaccatggtgagcaagggcgaggagctgttcaccggggtggtgcccatcctggtcgagctgg
acggcgacgtaaacggccacaagttcagcgtgtccggcgagggcgagggcgatgccacctacggcaagctgaccctgaagttc
atctgcaccaccggcaagctgcccgtgccctggcccaccctcgtgaccaccctgacctacggcgtgcagtgcttcagccgctacc
ccgaccacatgaagcagcacgacttcttcaagtccgccatgcccgaaggctacgtccaggagcgcaccatcttcttcaaggacga
cggcaactacaagacccgcgccgaggtgaagttcgagggcgacaccctggtgaaccgcatcgagctgaagggcatcgacttca
aggaggacggcaacatcctggggcacaagctggagtacaactacaacagccacaacgtctatatcatggccgacaagcagaag
aacggcatcaaggtgaacttcaagatccgccacaacatcgaggacggcagcgtgcagctcgccgaccactaccagcagaacac
ccccatcggcgacggccccgtgctgctgcccgacaaccactacctgagcacccagtccgccctgagcaaagaccccaacgaga
agcgcgatcacatggtcctgctggagttcgtgaccgccgccgggatcactctcggcatggacgagctgtacaagtaaagcggcc
gcgtcgaaaatgaattcgagctcggtacccccgggtacaaatcaattcactcctcaggtgcaggctgcctatcagaaggtggtggc
tggtgtggccaatgccctggctcacaaataccactgagatctttttccctctgccaaaaattatggggacatcatgaagccccttgag
catctgacttctggctaataaaggaaatttattttcattgcaatagtgtgttggaattttttgtgtctctcactcggaaggacatatgggag
ggcaaatcatttaaaacatcagaatgagtatttggtttagagtttggcaacatatgccatatgctggctgccatgaacaaaggtggcta
taaagaggtcatcagtatatgaaacagccccctgctgtccattccttattccatagaaaagccttgacttgaggttagattttttttatattt
tgttttgtgttatttttttctttaacatccctaaaattttccttacatgttttactagccagatttttcctcctctcctgactactcccagtcatagc
tgtccctcttctcttatgaagatccctcgacctgcagcccaagcttttcatacaccacaaatcgaggctgtagctggggcctttaacatt
gcagtttttttattcttcagtacactttgttgattctttgccttgatcttgacttcaggttctatcaccaccccctcagatggtgttccacactt
gggcctattcacagttcagagagctttacaacaatagatgtattgagaatccaacctaaagttcagctttttactcccatgaatgcctctt
tcctttttctccatttataaactgagccatttcctgttaatggtttacagatgaatatctcctcccccaatatcacctgatgtatcttacatttt
gccaggcttagattgtcttaaaaggtacataaattaacatgtgaaatttactccttaatgcttcagtggatttcatgagtgcagtacagaa
gactggtaatgggctaataacttttatttcattatttctcatatactcacttaactcttgagctacatggaattgattcctgcttactaaaatc
attatactcctctataaaagttagttccttctggaatgcagaatatataaactcttaaaggtttagttgtttgtctttcctgacctaaggtcca
gtgagcctgtatttttttctatttaagcggtgctttctcttggactggcttgactcatgttcatgttattgctgatttaaatgtgattttgctaag
tatcttctggacataattttgcttgacttgttgccagacacaagtaaaatggagtaagcagcaaaaatgtcctaggg
SEQ ID NO. 48 - Nucleic acid sequence of the AAVS1 donor
tgctttctctgaccagcattctctcccctgggcctgtgccgctttctgtctgcagcttgtggcctgggtcacctctacggctggcccag
atccttccctgccgcctccttcaggttccgtcttcctccactccctcttccccttgctctctgctgtgttgctgcccaaggatgctctttcc
ggagcacttccttctcggcgctgcaccacgtgatgtcctctgagcggatcctccccgtgtctgggtcctctccgggcatctctcctcc
ctcacccaaccccatgccgtcttcactcgctgggttcccttttccttctccttctggggcctgtgccatctctcgtttcttaggatggcctt
ctccgacggatgtctcccttgcgtcccgcctccccttcttgtaggcctgcatcatcaccgtttttctggacaaccccaaagtaccccgt
ctccctggctttagccacctctccatcctcttgctttctttgcctggacaccccgttctcctgtggattcgggtcacctctcactcctttcat
ttgggcagctcccctaccccccttacctctctagtctgtgctagctcttccagccccctgtcatggcatcttccaggggtccgagagct
cagctagtcttcttcctccaacccgggcccctatgtccacttcaggacagcatgtttgctgcctccagggatcctgtgtccccgagct
gggaccaccttatattcccagggccggttaatgtggctctggttctgggtacttttatctgtcccctccaccccacagtggggcaagc
ttctgacctcttctcttcctcccacagggcctcgagagatctggcagcggagagggcagaggaagtcttctaacatgcggtgacgt
ggaggagaatcccggccctaggctcgagatggtgagcaagggcgaggagctgttcaccggggtggtgcccatcctggtcgagc
tggacggcgacgtaaacggccacaagttcagcgtgtccggcgagggcgagggcgatgccacctacggcaagctgaccctgaa
gttcatctgcaccaccggcaagctgcccgtgccctggcccaccctcgtgaccaccctgacctacggcgtgcagtgcttcagccgc
taccccgaccacatgaagcagcacgacttcttcaagtccgccatgcccgaaggctacgtccaggagcgcaccatcttcttcaagg
acgacggcaactacaagacccgcgccgaggtgaagttcgagggcgacaccctggtgaaccgcatcgagctgaagggcatcga
cttcaaggaggacggcaacatcctggggcacaagctggagtacaactacaacagccacaacgtctatatcatggccgacaagca
gaagaacggcatcaaggtgaacttcaagatccgccacaacatcgaggacggcagcgtgcagctcgccgaccactaccagcaga
acacccccatcggcgacggccccgtgctgctgcccgacaaccactacctgagcacccagtccgccctgagcaaagaccccaac
gagaagcgcgatcacatggtcctgctggagttcgtgaccgccgccgggatcactctcggcatggacgagctgtacaagtaaagc
ggccgcgtcgagtctagagggcccgtttaaacccgctgatcagcctcgactgtgccttctagttgccagccatctgttgtttgcccct
cccccgtgccttccttgaccctggaaggtgccactcccactgtcctttcctaataaaatgaggaaattgcatcgcattgtctgagtagg
tgtcattctattctggggggtggggtggggcaggacagcaagggggaggattgggaagacaatagcaggcatgctggggatgc
ggtgggctctatggaagctttactagggacaggattggtgacagaaaagccccatccttaggcctcctccttcctagtctcctgatatt
gggtctaacccccacctcctgttaggcagattccttatctggtgacacacccccatttcctggagccatctctctccttgccagaacct
ctaaggtttgcttacgatggagccagagaggatcctgggagggagagcttggcagggggtgggagggaagggggggatgcgt
gacctgcccggttctcagtggccaccctgcgctaccctctcccagaacctgagctgctctgacgcggctgtctggtgcgtttcactg
atcctggtgctgcagcttccttacacttcccaagaggagaagcagtttggaaaaacaaaatcagaataagttggtcctgagttctaac
tttggctcttcacctttctagtccccaatttatattgttcctccgtgcgtcagttttacctgtgagataaggccagtagccagccccgtcct
ggcagggctgtggtgaggaggggggtgtccgtgtggaaaactccctttgtgagaatggtgcgtcctaggtgttcaccaggtcgtg
gccgcctctactccctttctctttctccatccttctttccttaaagagtccccagtgctatctgggacatattcctccgcccagagcaggg
tcccgcttccctaaggccctgctctgggcttctgggtttgagtccttggcaagcccaggagaggcgctcaggcttccctgtccccctt
cctcgtccaccatctcatgcccctggctctcctgccccttccctacaggggttcctggctctgctctaa
SEQ ID NO. 49 - Nucleic acid sequence of the region of the JAK gene targeted by the
spacer 54
tccaggttcacctcagtcttcttggagctcctcattttag
SEQ ID NO. 50 - Nucleic acid sequence of the PCR product obtained in the region of
the JAK gene targeted by the spacer 54
tccaggttcacctcagttcttcttggagctcctcattttag
SEQ ID NO. 51 - Nucleic acid sequence of the PCR product obtained in the region of
the JAK gene targeted by the spacer 54
tccaggttcacctcagttcttggagctcctcattttag
SEQ ID NO. 52 - Nucleic acid sequence of the PCR product obtained in the region of
the JAK gene targeted by the spacer 54
tccaggttcacctcttcttggagctcctcattttag
SEQ ID NO. 53 - Nucleic acid sequence of the PCR product obtained in the region of
the JAK gene targeted by the spacer 54
tccaggttcacctcattttag
SEQ ID NO. 54 - Nucleic acid sequence of the PCR product obtained in the region of
the JAK gene targeted by the spacer 54
tccaggttcacctcagcttcttggagctcctcattttag
SEQ ID NO. 55 - Nucleic acid sequence of the PCR product obtained in the region of
the JAK gene targeted by the spacer 54
tccaggttcacctcagtcttggagctcctcattttag
SEQ ID NO. 56 - Nucleic acid sequence of the PCR product obtained in the region of
the JAK gene targeted by the spacer 54
tccaggttcaccttcttggagctcctcattttag
SEQ ID NO. 57 - Nucleic acid sequence of the PCR product obtained in the region of
the JAK gene targeted by the spacer 54
tccaggttcttggagctcctcattttag
SEQ ID NO. 58 - Nucleic acid sequence of the PCR product obtained in the region of
the JAK gene targeted by the spacer 54
tccaggttcacctcttggagctcctcattttag
SEQ ID NO. 59 - Nucleic acid sequence of the region of the PCSK gene targeted by
the spacer 93
gagctttaaaatggttccgacttgtccctctctcagccctc
SEQ ID NO. 60 - Nucleic acid sequence of the PCR product obtained in the region of
the PCSK gene targeted by the spacer 93
gagctttaaaatggttccgactttgtccctctctcagccctc
SEQ ID NO. 61 - Nucleic acid sequence of the PCR product obtained in the region of
the PCSK gene targeted by the spacer 93
gagctttaaaatggtccctctctcagccctc
SEQ ID NO. 62 - Nucleic acid sequence of the PCR product obtained in the region of
the PCSK gene targeted by the spacer 93
gagctttaaaatggttccgactgtccctctctcagccctc
SEQ ID NO. 63 - Nucleic acid sequence of the PCR product obtained in the region of
the PCSK gene targeted by the spacer 93
gagctttaaaatggttccgactctcagccctc
SEQ ID NO. 64 - Nucleic acid sequence of the PCR product obtained in the region of
the PCSK gene targeted by the spacer 93
gagctttaaaatggttccctctctcagccctc
SEQ ID NO. 65 - Nucleic acid sequence of the PCR product obtained in the region of
the PCSK gene targeted by the spacer 93
gagctttaaaatggttc
SEQ ID NO. 66 - Nucleic acid sequence of the PCR product obtained in the region of
the PCSK gene targeted by the spacer 93
gagctttaaaatgtccctctctcagccctc
SEQ ID NO. 67 - Nucleic acid sequence of the region of the AAVS1 locus targeted by
the T2 guide RNA
aaggatggggcttttctgtcaccaatcctgtccctagtggc
SEQ ID NO. 68 - Nucleic acid sequence of the PCR product obtained in the region of
the AAVS1 locus targeted by the T2 guide RNA
aaggatggggcttttctgtcaccaatctgtccctagtggc
SEQ ID NO. 69 - Nucleic acid sequence of the PCR product obtained in the region of
the AAVS1 locus targeted by the T2 guide RNA
aaggatggggcttttctgtcaccaatccgtccctagtggc
SEQ ID NO. 70 - Nucleic acid sequence of the PCR product obtained in the region of
the AAVS1 locus targeted by the T2 guide RNA
aaggatggggcttttctgtccctagtggc
SEQ ID NO. 71 - Nucleic acid sequence of the PCR product obtained in the region of
the AAVS1 locus targeted by the T2 guide RNA
aaggatggggcttttctgtcaccaatccctgtccctagtggc
SEQ ID NO. 72 - Nucleic acid sequence of the PCR product obtained in the region of
the AAVS1 locus targeted by the T2 guide RNA
aaggatggggcttttctgtcaccaatccctagtggc
SEQ ID NO. 73 - Nucleic acid sequence of the PCR product obtained in the region of
the AAVS1 locus targeted by the T2 guide RNA
aaggatggggcttttctgtcaccaatcgtccctagtggc
SEQ ID NO. 74 - Nucleic acid sequence of the PCR product obtained in the region of
the AAVS1 locus targeted by the T2 guide RNA
aaggatggggcttttctgtcaccaatcctgctgtccctagtggc
SEQ ID NO. 75 - Nucleic acid sequence of the PCR product obtained in the region of
the AAVS1 locus targeted by the T2 guide RNA
aaggatggggcttttctgtcacctagtggc
SEQ ID NO. 76 - Nucleic acid sequence of the siRNA siNT
augaacgugaauugcucaa(dtdt)
SEQ ID NO. 77 - Nucleic acid sequence of the siRNA siCtIP
gcuaaaacaggaacgaauc
SEQ ID NO. 78 - Nucleic acid sequence of the spacer sequence of the T2 guide RNA
ggggccacuagggacaggau
SEQ ID NO. 79 - Nucleic acid sequence of the target sequence of the T2 guide RNA
ggggccactagggacaggattgg
SEQ ID NO. 80 - Nucleic acid sequence of the spacer sequence of the T4 guide RNA
gacagaaaagccccauccuuuu
SEQ ID NO. 81 - Nucleic acid sequence of the target sequence of guide T4 RNA
gacagaaaagccccatccttttggg
SEQ ID NO. 82 - Nucleic acid sequence of T4 guide RNA
gacagaaaagccccatccttttgttttagagctagaaatagcaagttaaaataaggctagtccgttatcaacttgaaaaagtggcacc
gagtcggtgc
SEQ ID NO. 83 - Nucleic acid sequence of the spacer sequence of D1 guide RNA
gacuaggaaggguuagacccaaaagga
SEQ ID NO. 84 - Nucleic acid sequence of the target sequence of the D1 guide RNA
gactaggaagggttagacccaaaaggatgg
SEQ ID NO. 85 - Nucleic acid sequence of the D1 guide RNA
gactaggaagggttagacccaaaaggagttttagagctagaaatagcaagttaaaataaggctagtccgttatcaacttgaaaaagt
ggcaccgagtcggtgc

1-17. (canceled)

18. A fusion protein comprising at least (a) a nuclease, (b) a dimerization domain of a CtIP protein and (c) a tetramerization domain of a CtIP protein, with the proviso that the fusion protein does not comprise a full length CtIP protein.

19. The fusion protein according to claim 1, wherein the nuclease is selected from the group consisting of a Cas nuclease, a zinc-finger nuclease (ZFN), transcription-activator like effector nuclease (TALEN) and a meganuclease.

20. The fusion protein according to claim 18, wherein the nuclease is a Cas nuclease.

21. The fusion protein according to claim 20, wherein the Cas nuclease is a Cas9 nuclease.

22. The fusion protein according to claim 18, which further comprises a domain of a CtIP protein comprising at least one cyclin-dependent kinase (CDK) phosphorylation site.

23. The fusion protein according to claim 22, wherein the at least one CDK phosphorylation site comprises a serine to glutamic acid (Ser/Glu) or a threonine to glutamic acid (Thr/Glu) substitution.

24. The fusion protein according to claim 18, which further comprises a nuclear localization domain.

25. The fusion protein according to claim 18, wherein the CtIP protein is of human origin.

26. A nucleic acid encoding a fusion protein according to claim 18.

27. A nucleic acid vector for recombinant protein expression comprising a nucleic acid according to claim 26.

28. A delivery particle comprising a fusion protein according to claim 18, a nucleic acid encoding the fusion protein or a nucleic acid vector comprising the nucleic acid.

29. The delivery particle according to claim 28, which further comprises at its surface one or more targeting ligands suitable for specifically addressing said delivery particle to a targeted cell.

30. A method for treating a genetic disorder, a cancer and/or an infectious disease comprising the step of administering to an individual in need thereof of
a fusion protein according to claim 18;
a nucleic acid encoding the fusion protein;
a nucleic acid vector comprising the nucleic acid; or
a delivery particle comprising the fusion protein, the nucleic acid or the nucleic acid vector.

31. A host cell comprising
a fusion protein according to claim 18,
a nucleic acid encoding the fusion protein; or
a nucleic acid vector comprising the nucleic acid.

32. A pharmaceutical composition comprising
(i) a fusion protein according to claim 18;
a nucleic acid encoding the fusion protein;
a nucleic acid vector comprising the nucleic acid; or
a delivery particle comprising the fusion protein, the nucleic acid or the nucleic acid vector, and
(ii) a pharmaceutically acceptable vehicle.

33. A method for editing a genome in at least one target cell comprising the step of administering to an individual in need thereof a pharmaceutical composition according to claim 32.

34. Kit for editing the genome of at least one target cell, comprising:
(i) a fusion protein according to claim 18;
a nucleic acid encoding the fusion protein;
a nucleic acid vector comprising the nucleic acid; or
a delivery particle comprising the fusion protein, the nucleic acid or the nucleic acid vector; and
(ii) one or more site-specific guide RNAs (gRNAs) or a nucleic acid vector for expressing the one or more site specific guide RNAs (gRNAs).


SEQUENCE LISTING (85 sequences)

SEQ ID NO: 1 - protein, 1372 aa, Streptococcus pyogenes
  Cas 9 nuclease
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRAD

SEQ ID NO: 2 - protein, 897 aa, Homo sapiens
  CtIP protein
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 3 - protein, 24 aa, Artificial Sequence
  tetramerization domain of human CtIP (22-45)
DLWTKLKECHDREVQGLQVKVTKL

SEQ ID NO: 4 - protein, 121 aa, Artificial Sequence
  dimerization domain of human CtIP (46-166)
KQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPIT

SEQ ID NO: 5 - protein, 149 aa, Artificial Sequence
  1-149 domain of human CtIP
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQ

SEQ ID NO: 6 - protein, 296 aa, Artificial Sequence
  1-296 (HE) domain of human CtIP
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFE

SEQ ID NO: 7 - protein, 416 aa, Artificial Sequence
  1-416 domain of human CtIP
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESL

SEQ ID NO: 8 - protein, 669 aa, Artificial Sequence
  1-669 domain of CtIP
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMD

SEQ ID NO: 9 - protein, 790 aa, Artificial Sequence
  1-790 domain of human CtIP (delta?SD)
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSL

SEQ ID NO: 10 - protein, 482 aa, Artificial Sequence
  416-897 domain of human CtIP
LGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 11 - protein, 229 aa, Artificial Sequence
  669-897 domain of human CtIP
DVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 12 - protein, 170 aa, Artificial Sequence
  1-170 domain of human CtIP (HE1)
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSF

SEQ ID NO: 13 - protein, 251 aa, Artificial Sequence
  46-296 domain of human CtIP (HE2)
KQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFE

SEQ ID NO: 14 - protein, 131 aa, Artificial Sequence
  166-296 domain of human CtIP (HE3)
TAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFE

SEQ ID NO: 15 - protein, 296 aa, Artificial Sequence
  HE(3E) domain of human CtIP
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQEPMAKAHGTSSYEPDKSSFNLATVVAETLGLGVQEESETQGPMEPLGDELYHCLEGNHKKQPFE

SEQ ID NO: 16 - protein, 296 aa, Artificial Sequence
  HE(3A) domain of human CtIP
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQAPMAKAHGTSSYAPDKSSFNLATVVAETLGLGVQEESETQGPMAPLGDELYHCLEGNHKKQPFE

SEQ ID NO: 17 - protein, 7 aa, Artificial Sequence
  SV40 NLS1
PKKKRKV

SEQ ID NO: 18 - protein, 16 aa, Artificial Sequence
  NLS of nucleoplasmin
KRPAATKKAGQAKKKK

SEQ ID NO: 19 - protein, 9 aa, Artificial Sequence
  NLS of c-Myc
PAAKRVKLD

SEQ ID NO: 20 - protein, 25 aa, Artificial Sequence
  NLS of EGL-13
MSRRRKANPTKLSENAKKLAKEVEN

SEQ ID NO: 21 - protein, 2278 aa, Artificial Sequence
  fusion protein Cas9-human CtIP
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVGSMNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 22 - protein, 1679 aa, Artificial Sequence
  Fusion protein Cas9-HE domain of human CtIP
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVGSASMNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFE

SEQ ID NO: 23 - protein, 1553 aa, Artificial Sequence
  Fusion protein Cas9-HE1 domain of human CtIP
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVGSASMNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSF

SEQ ID NO: 24 - protein, 1679 aa, Artificial Sequence
  Fusion protein Cas9-HE(3E) domain of human CtIP
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVGSASMNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQEPMAKAHGTSSYEPDKSSFNLATVVAETLGLGVQEESETQGPMEPLGDELYHCLEGNHKKQPFE

SEQ ID NO: 25 - DNA, 4116 bp, Streptococcus pyogenes
  Nucleic acid sequence of Cas9 of S. pyogenes
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgac

SEQ ID NO: 26 - DNA, 2694 bp, Homo sapiens
  Nucleic acid sequence of human CtIP
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatag

SEQ ID NO: 27 - DNA, 888 bp, Artificial Sequence
  Nucleic acid sequence of HE domain of the human CtIP
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgag

SEQ ID NO: 28 - DNA, 888 bp, Artificial Sequence
  Nucleic acid sequence of HE(3E) domain of the human CtIP
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaagagccaatggccaaagcacatggaacaagcagctatgaacctgataagtcatcttttaatttagctacagttgttgctgaaacacttggacttggtgttcaagaagaatctgaaactcaaggtcccatggaaccccttggtgatgagctctaccactgtctggaaggaaatcacaagaaacagccttttgag

SEQ ID NO: 29 - DNA, 7833 bp, Artificial Sequence
  Nucleic acid of a Cas9-human CtIP fusion
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggatccatgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatagaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagc

SEQ ID NO: 30 - DNA, 6036 bp, Artificial Sequence
  Nucleic acid of a Cas9-HE domain of the human CtIP fusion
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggatccgctagcatgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgagtagaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagc

SEQ ID NO: 31 - DNA, 6036 bp, Artificial Sequence
  Nucleic acid of a Cas9-HE(3E) domain of the human CtIP fusion
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggatccgctagcatgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaagagccaatggccaaagcacatggaacaagcagctatgaacctgataagtcatcttttaatttagctacagttgttgctgaaacacttggacttggtgttcaagaagaatctgaaactcaaggtcccatggaaccccttggtgatgagctctaccactgtctggaaggaaatcacaagaaacagccttttgagtagaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagc

SEQ ID NO: 32 - DNA, 20 bp, Artificial Sequence
  Nucleic acid sequence of the T7AAVFw primer
cagcaccaggatcagtgaaa

SEQ ID NO: 33 - DNA, 22 bp, Artificial Sequence
  Nucleic acid sequence of the T7AAVRev primer
ctatgtccacttcaggacagca

SEQ ID NO: 34 - DNA, 21 bp, Artificial Sequence
  Nucleic acid sequence of the rROSA-5HAFor primer
ttcttccacttgcgatccttg

SEQ ID NO: 35 - DNA, 25 bp, Artificial Sequence
  Nucleic acid sequence of the 5CAGpRev primer
ggctatgaactaatgaccccgtaat

SEQ ID NO: 36 - DNA, 22 bp, Artificial Sequence
  Nucleic acid sequence of the 3BGHpA-Up2 primer
ccagatttttcctcctctcctg

SEQ ID NO: 37 - DNA, 23 bp, Artificial Sequence
  Nucleic acid sequence of the rROSAfw1 primer
tgaactgtgaataggcccaagtg

SEQ ID NO: 38 - DNA, 20 bp, Artificial Sequence
  Nucleic acid sequence of the rROSA26-5outFor primer
tcccaccctccccttcctct

SEQ ID NO: 39 - DNA, 25 bp, Artificial Sequence
  Nucleic acid sequence of the 5CAGpRev primer
ggctatgaactaatgaccccgtaat

SEQ ID NO: 40 - DNA, 22 bp, Artificial Sequence
  Nucleic acid sequence of the 3BGHpA-Up2 primer
ccagatttttcctcctctcctg

SEQ ID NO: 41 - DNA, 25 bp, Artificial Sequence
  Nucleic acid sequence of the rROSA26-3outRev primer
tgggtatcactggctgtcctagata

SEQ ID NO: 42 - DNA, 26 bp, Artificial Sequence
  Nucleic acid sequence of the rROSArev1 primer
gcattttaaaagagcccagtacttca

SEQ ID NO: 43 - DNA, 72 bp, Artificial Sequence
  Nucleic acid sequence of the tetramerization domain of human CtIP
gacctttggacaaaactaaaagaatgtcatgatagagaagtacaaggtttacaagtaaaagtaaccaagcta

SEQ ID NO: 44 - DNA, 363 bp, Artificial Sequence
  Nucleic acid sequence of the dimerization domain of human CtIP
aaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataaca

SEQ ID NO: 45 - DNA, 393 bp, Artificial Sequence
  Nucleic acid sequence of the HE3 domain of human CtIP
acagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgag

SEQ ID NO: 46 - DNA, 96 bp, Artificial Sequence
  Nucleic acid sequence of the gRNA for targeting the AAVS1 safe harbor locus
ggggccactagggacaggatgttttagagctagaaatagcaagttaaaataaggctagtccgttatcaacttgaaaaagtggcaccgagtcggtgc

SEQ ID NO: 47 - DNA, 4697 bp, Artificial Sequence
  Nucleic acid sequence of the rat ROSA donor sequence
gtaacccctagggagttggggctcagtcgggttgtattggagacaagaagcacttgctctccaaaagtcggtttgagttatcattaagggagctgcagtggagtaggcggagaaaaggccgcacccttctcaggacgggggaggggagtgttgcaatacctttctgggagttctctgctgcctcctgtcttctgaggaccgccctgggcctggaagattcccttcccccttcttccctcgtgatctgcaactggagtctttctggaagataggcgggagtcttctgggcaggcttaaaggctaacctggtgcgtggggcgttgtcctgcagaggaattgaacaggtgtaaaattggaggggcaagacttcccacagattttcgattgtgttgttaagtattgtaataggggcaaataagggaaatagactaggcactcacctggggttttatgcagcaaaactacaggttattattgcttgtgatccgccctggagaatttttcaccgaggtagattgaagacatgcccacccaaattttaatattcttccacttgcgatccttgctacagtatgaaattacagtatcgtgaattagaatatataagcagaattttaagcattttaaaagagcccagtacttcatgtctgtctctcccacttctgcagccctatcaaagggtattttagcacactcattttagtcccattttcatttgttgtactggcttatccaatccctagacagagcactggcattccctctctcctgatcttagaagtccgatgactcatgaaaccagacagattagtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgattaattcgagcgaacgcgtcgagtcgctcggtacgatttgtaatttgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgaaaatgaattcgagctcggtacccccgggtacaaatcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttttcatacaccacaaatcgaggctgtagctggggcctttaacattgcagtttttttattcttcagtacactttgttgattctttgccttgatcttgacttcaggttctatcaccaccccctcagatggtgttccacacttgggcctattcacagttcagagagctttacaacaatagatgtattgagaatccaacctaaagttcagctttttactcccatgaatgcctctttcctttttctccatttataaactgagccatttcctgttaatggtttacagatgaatatctcctcccccaatatcacctgatgtatcttacattttgccaggcttagattgtcttaaaaggtacataaattaacatgtgaaatttactccttaatgcttcagtggatttcatgagtgcagtacagaagactggtaatgggctaataacttttatttcattatttctcatatactcacttaactcttgagctacatggaattgattcctgcttactaaaatcattatactcctctataaaagttagttccttctggaatgcagaatatataaactcttaaaggtttagttgtttgtctttcctgacctaaggtccagtgagcctgtatttttttctatttaagcggtgctttctcttggactggcttgactcatgttcatgttattgctgatttaaatgtgattttgctaagtatcttctggacataattttgcttgacttgttgccagacacaagtaaaatggagtaagcagcaaaaatgtcctaggg

SEQ ID NO: 48 - DNA, 2765 bp, Artificial Sequence
  Nucleic acid sequence of the AAVS1 donor
tgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggagagggcagaggaagtcttctaacatgcggtgacgtggaggagaatcccggccctaggctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaa

SEQ ID NO: 49 - DNA, 40 bp, Artificial Sequence
  Nucleic acid sequence of the region of the JAK gene targeted by the spacer 54
tccaggttcacctcagtcttcttggagctcctcattttag

SEQ ID NO: 50 - DNA, 41 bp, Artificial Sequence
  Nucleic acid sequence of the PCR product obtained in the region of the JAK gene targeted by the spacer 54
tccaggttcacctcagttcttcttggagctcctcattttag

SEQ ID NO: 51 - DNA, 38 bp, Artificial Sequence
  Nucleic acid sequence of the PCR product obtained in the region of the JAK gene targeted by the spacer 54
tccaggttcacctcagttcttggagctcctcattttag

SEQ ID NO: 52 - DNA, 36 bp, Artificial Sequence
  Nucleic acid sequence of the PCR product obtained in the region of the JAK gene targeted by the spacer 54
tccaggttcacctcttcttggagctcctcattttag

SEQ ID NO: 53 - DNA, 21 bp, Artificial Sequence
  Nucleic acid sequence of the PCR product obtained in the region of the JAK gene targeted by the spacer 54
tccaggttcacctcattttag

SEQ ID NO: 54 - DNA, 39 bp, Artificial Sequence
  Nucleic acid sequence of the PCR product obtained in the region of the JAK gene targeted by the spacer 54
tccaggttcacctcagcttcttggagctcctcattttag

SEQ ID NO: 55 - DNA, 37 bp, Artificial Sequence
  Nucleic acid sequence of the PCR product obtained in the region of the JAK gene targeted by the spacer 54
tccaggttcacctcagtcttggagctcctcattttag

SEQ ID NO: 56 - DNA, 34 bp, Artificial Sequence
  Nucleic acid sequence of the PCR product obtained in the region of the JAK gene targeted by the spacer 54
tccaggttcaccttcttggagctcctcattttag

SEQ ID NO: 57 - DNA, 28 bp, Artificial Sequence
  Nucleic acid sequence of the PCR product obtained in the region of the JAK gene targeted by the spacer 54
tccaggttcttggagctcctcattttag

SEQ ID NO: 58 - DNA, 33 bp, Artificial Sequence
  Nucleic acid sequence of the PCR product obtained in the region of the JAK gene targeted by the spacer 54
tccaggttcacctcttggagctcctcattttag

SEQ ID NO: 59 - DNA, 41 bp, Artificial Sequence
  acid sequence of the region of the PCSK gene targeted by the spacer 93
gagctttaaaatggttccgacttgtccctctctcagccctc

SEQ ID NO: 60 - DNA, 42 bp, Artificial Sequence
  Nucleic acid sequence of the PCR product obtained in the region of the PCSK gene targeted by the spacer 93
gagctttaaaatggttccgactttgtccctctctcagccctc

SEQ ID NO: 61 - DNA, 31 bp, Artificial Sequence
  Nucleic acid sequence of the PCR product obtained in the region of the PCSK gene targeted by the spacer 93
gagctttaaaatggtccctctctcagccctc

SEQ ID NO: 62 - DNA, 40 bp, Artificial Sequence
  Nucleic acid sequence of the PCR product obtained in the region of the PCSK gene targeted by the spacer 93
gagctttaaaatggttccgactgtccctctctcagccctc

SEQ ID NO: 63 - DNA, 32 bp, Artificial Sequence
  Nucleic acid sequence of the PCR product obtained in the region of the PCSK gene targeted by the spacer 93
gagctttaaaatggttccgactctcagccctc

SEQ ID NO: 64 - DNA, 32 bp, Artificial Sequence
  Nucleic acid sequence of the PCR product obtained in the region of the PCSK gene targeted by the spacer 93
gagctttaaaatggttccctctctcagccctc

SEQ ID NO: 65 - DNA, 17 bp, Artificial Sequence
  Nucleic acid sequence of the PCR product obtained in the region of the PCSK gene targeted by the spacer 93
gagctttaaaatggttc

SEQ ID NO: 66 - DNA, 30 bp, Artificial Sequence
  Nucleic acid sequence of the PCR product obtained in the region of the PCSK gene targeted by the spacer 93
gagctttaaaatgtccctctctcagccctc

SEQ ID NO: 67 - DNA, 41 bp, Artificial Sequence
  Nucleic acid sequence of the region of the AAVS1 locus targeted by the T2 guide RNA
aaggatggggcttttctgtcaccaatcctgtccctagtggc

SEQ ID NO: 68 - DNA, 40 bp, Artificial Sequence
  Nucleic acid sequence of the PCR product obtained in the region of the AAVS1 locus targeted by the T2 guide RNA
aaggatggggcttttctgtcaccaatctgtccctagtggc

SEQ ID NO: 69 - DNA, 40 bp, Artificial Sequence
  Nucleic acid sequence of the PCR product obtained in the region of the AAVS1 locus targeted by the T2 guide RNA
aaggatggggcttttctgtcaccaatccgtccctagtggc

SEQ ID NO: 70 - DNA, 29 bp, Artificial Sequence
  Nucleic acid sequence of the PCR product obtained in the region of the AAVS1 locus targeted by the T2 guide RNA
aaggatggggcttttctgtccctagtggc

SEQ ID NO: 71 - DNA, 42 bp, Artificial Sequence
  Nucleic acid sequence of the PCR product obtained in the region of the AAVS1 locus targeted by the T2 guide RNA
aaggatggggcttttctgtcaccaatccctgtccctagtggc

SEQ ID NO: 72 - DNA, 36 bp, Artificial Sequence
  Nucleic acid sequence of the PCR product obtained in the region of the AAVS1 locus targeted by the T2 guide RNA
aaggatggggcttttctgtcaccaatccctagtggc

SEQ ID NO: 73 - DNA, 39 bp, Artificial Sequence
  Nucleic acid sequence of the PCR product obtained in the region of the AAVS1 locus targeted by the T2 guide RNA
aaggatggggcttttctgtcaccaatcgtccctagtggc

SEQ ID NO: 74 - DNA, 44 bp, Artificial Sequence
  Nucleic acid sequence of the PCR product obtained in the region of the AAVS1 locus targeted by the T2 guide RNA
aaggatggggcttttctgtcaccaatcctgctgtccctagtggc

SEQ ID NO: 75 - DNA, 30 bp, Artificial Sequence
  Nucleic acid sequence of the PCR product obtained in the region of the AAVS1 locus targeted by the T2 guide RNA
aaggatggggcttttctgtcacctagtggc

SEQ ID NO: 76 - RNA, 21 nt, Artificial Sequence
  Nucleic acid sequence of the siRNA siNT
  misc_feature 20,21: /note=“the nucleotides in positions 20 and 21 represent deoxythymidine nucleotides”
augaacgugaauugcucaatt

SEQ ID NO: 77 - RNA, 19 nt, Artificial Sequence
  Nucleic acid sequence of the siRNA siCtIP
gcuaaaacaggaacgaauc

SEQ ID NO: 78 - RNA, 20 nt, Artificial Sequence
  Nucleic acid sequence of the spacer sequence of the T2 guide RNA
ggggccacuagggacaggau

SEQ ID NO: 79 - DNA, 23 bp, Artificial Sequence
  Nucleic acid sequence of the target sequence of the T2 guide RNA
ggggccactagggacaggattgg

SEQ ID NO: 80 - RNA, 22 nt, Artificial Sequence
  Nucleic acid sequence of the spacer sequence of the T4 guide RNA
gacagaaaagccccauccuuuu

SEQ ID NO: 81 - DNA, 25 bp, Artificial Sequence
  Nucleic acid sequence of the target sequence of guide T4 RNA
gacagaaaagccccatccttttggg

SEQ ID NO: 82 - DNA, 98 bp, Artificial Sequence
  Nucleic acid sequence of T4 guide RNA
gacagaaaagccccatccttttgttttagagctagaaatagcaagttaaaataaggctagtccgttatcaacttgaaaaagtggcaccgagtcggtgc

SEQ ID NO: 83 - RNA, 27 nt, Artificial Sequence
  Nucleic acid sequence of the spacer sequence of D1 guide RNA
gacuaggaaggguuagacccaaaagga

SEQ ID NO: 84 - DNA, 30 bp, Artificial Sequence
  Nucleic acid sequence of the target sequence of the D1 guide RNA
gactaggaagggttagacccaaaaggatgg

SEQ ID NO: 85 - DNA, 103 bp, Artificial Sequence
  Nucleic acid sequence of the D1 guide RNA
gactaggaagggttagacccaaaaggagttttagagctagaaatagcaagttaaaataaggctagtccgttatcaacttgaaaaagtggcaccgagtcggtgc